(19) United States
(12) Patent Application Publication
(10) Pub. No.: US 2025/0387479 A1
(43) Pub. Date: Dec. 25, 2025

(54) STABLE FORMULATIONS FOR ANTIBODIES

(71) Applicant: MABXIENCE RESEARCH, S.L., Madrid (ES)

(72) Inventors: Ivan SANCHEZ MELO, Armunia (ES); Esther TEJEDA MONTES, Armunia (ES); Roshan GOSWAMI, Sagar (IN); Sergio GUTIERREZ GETINO, Armunia (ES)

(21) Appl. No.: 18/844,657

(22) PCT Filed: Mar. 6, 2023
(86) PCT No.: PCT/EP2023/055569

§ 371 Date: Sep. 6, 2024

(30) Foreign Application Priority Data
Mar. 7, 2022 (EP) 22382206.5

(51) Int. Cl. A61K 39/395; A61K 47/12; A61K 47/18; A61K 47/26; A61K 47/34; C07K 16/10; C07K 16/28

(52) U.S. Cl. CPC A61K 39/39591; A61K 47/12; A61K 47/183; A61K 47/26; A61K 47/34; C07K 16/10; C07K 16/2818; C07K 2317/24

(57) ABSTRACT

The disclosure relates to a formulation comprising an antibody, a buffer, an amino acid and trehalose, having a pH between 5.0 to 7.0. The formulation of the disclosure finds application in the prevention and/or treatment of a disease which requires neutralization of the antigen which is targeted by the antibody present in the formulation. The disclosure also relates to a method for preparing a stabilized antibody-containing composition.

### FIELD OF THE INVENTION

The present disclosure relates to medicines for the treatment of diseases and, more specifically, to stable formulations for therapeutic proteins.

### BACKGROUND OF THE INVENTION

Therapeutic proteins, with higher specificity towards targets and superior safety than small molecule therapeutics, have increasingly become part of the repertoire of drugs available to medical practitioners for the treatment of a wide range of pathologies from cancer to infectious diseases. Long-term stability of a therapeutic protein is a particularly important criterion for safety and efficacy of a given treatment. Structural modifications and loss of functionality of a therapeutic protein within a preparation can severely affect the activity and/or the safety of a preparation, leading to loss of efficacy and risk of adverse side effects.

The formulation of proteins, which are larger and more complex than organic and inorganic drugs, however, poses special problems. A formulation must provide conditions for a protein to remain biologically active, thereby preserving its conformational integrity as well as protecting it from degradation. Structural complexity of biological pharmaceuticals, such as proteins, make them susceptible to chemical or physical alterations that result in structural and functional instability as well as loss of safety. Chemical instability may result from hydrolysis, oxidation, disulfide exchange, deamidation, et cetera, whereas physical instability may arise from aggregation, fragmentation, denaturation, precipitation, adsorption etc. The composition of a formulation can significantly affects the extent of protein degradation and, consequently, the safety and efficacy of the therapeutic, as well as the ease and frequency of administration.

Despite the advances with regards to therapeutic protein formulations, there is still a need to develop formulations with enhanced long-term stability features. A formulation that retains long-term stability under a variety of conditions would provide an effective means of delivering an efficacious and safe amount of a therapeutic protein, which in turn would result in lower production and treatment costs. Recombinant or natural therapeutic proteins could benefit from stable formulations and thereby provide more effective clinical results.

Thus, there exists a need for formulations that contribute to retain long-term stability in therapeutic proteins under diverse manufacturing and storage conditions.

### SUMMARY OF THE INVENTION

The authors of the present disclosure have found that the stability of antibody molecules at both a wide range of temperatures and low pH conditions can be enhanced by providing a formulation comprising an antibody, a buffer, an amino acid and trehalose, having a pH between 5.0 to 7.0.

Therefore, in a first aspect the disclosure relates to a formulation comprising an antibody, a buffer, an amino acid and trehalose, having a pH between 5.0 to 7.0.

In a second aspect, the disclosure relates to the formulation of the disclosure for use in the prevention and/or treatment of a disease caused by an infection by the Respiratory Syncytial Virus (RSV).

In a third aspect, the disclosure relates to the formulation of the disclosure for use in the prevention and/or treatment of cancer.

In a fourth aspect, the disclosure relates to the formulation of the disclosure for use in the prevention and/or treatment of a disease caused by increased levels of PD-L1.

In a fifth aspect, the present disclosure relates to a method for preparing a stabilized antibody-containing composition comprising formulating the antibody in a composition comprising a buffer, an amino acid and trehalose, and wherein the formulation has a pH of 5.0 to 7.0.

### BRIEF DESCRIPTION OF THE FIGURES

FIG. 1A and FIG. 1B. (A) Graph showing the % of high-molecular weight species or levels of aggregates measured by size exclusion chromatography (SEC) of Palivizumab antibody on F0 formulation, compared to F45 formulation when maintaining both formulations at a temperature of 40° C. for up to 2 weeks. (B) Graph showing the high-molecular weight change or levels of aggregates measured by SEC of Palivizumab antibody on F0 formulation, compared to F45 when maintaining both formulations at a temperature of 25° C. for up to 16 weeks.

FIG. 1C and FIG. 1D. (C) Graph showing the % of high-molecular weight species or levels of aggregates measured by SEC of Palivizumab antibody on F0 formulation compared to F45 when maintaining both formulations at a temperature between 2-8° C. for up to 36 weeks. (D) Graph showing the high-molecular weight change or levels of aggregates measured by SEC of Palivizumab in the F45 formulation at a temperature of −20° C. versus −80° C. up to 24 weeks.

FIG. 1E. Graph showing the % of high-molecular weight species (% HMW) or levels of aggregates measured by SEC of Palivizumab antibody on F0 formulation compared to F45 formulation when maintaining both formulations at a temperature of −80° C. for up to 52 weeks.

FIG. 1F. Graph showing the % of high-molecular weight species (% HMW) or levels of aggregates measured by SEC of Palivizumab antibody on F0 formulation compared to F45 formulation at initial time point (TO) compared to both formulations being frozen at-80° C. for 1 week followed by storage at a temperature of 25° C. for 24 weeks.

FIG. 2A and FIG. 2B. (A) Graph showing the % of acidic forms analyzed by ion exchange chromatography (IEX) of Palivizumab antibody on F0 formulation compared to F45 formulation when maintaining both formulations at a temperature of 40° C. for up to 2 weeks. (B) Graph showing the % of acidic forms of Palivizumab antibody on F0 formulation compared to F45 formulation when maintaining both formulations at a temperature of 25° C. for up to 12 weeks.

FIG. 2C and FIG. 2D. (C) Graph showing the % of acidic forms analyzed by IEX of Palivizumab antibody on F0 formulation compared to F45 formulation when maintaining both formulations at a temperature between 2-8° C. for up to 24 weeks. (D) Graph showing the % of acidic forms of Palivizumab antibody in the F45 formulation at a temperature of −20° C. versus −80° C. up to 24 weeks.

FIG. 3A and FIG. 3B. (A) Graph showing the % of basic forms analyzed by capillary isoelectric focusing (cIEF) of Palivizumab antibody on F0 formulation compared to F45 formulation when maintaining both formulations at a temperature of 40° C. for 2 weeks. (B) Graph showing the % of basic forms analyzed by cIEF of Palivizumab antibody on F0 formulation compared to F45 formulation when maintaining both formulations at a temperature of 25° C. for up to 12 weeks.

FIG. 3C and FIG. 3D. (C) Graph showing the % of basic forms analyzed by cIEF of Palivizumab antibody on F0 formulation compared to F45 formulation when maintaining both formulations at a temperature between 2-8° C. for up to 24 weeks. (D) Graph showing the % of basic forms analyzed by cIEF of Palivizumab antibody in the F45 formulation at a temperature of −20° C. versus −80° C. up to 24 weeks.

FIG. 4A. Graph showing the % of high-molecular weight species (% HMW) or levels of aggregates measured by SEC on Palivizumab antibody in F45 formulation when compared to Synagis®-EU & Synagis®-US when submitted to forced degradation conditions comprising high pH and low pH at 3 to 7 days, oxidation stress for 3, 7 and 14 days, agitation for 3 days and high temperature for 7 and 14 days.

FIG. 4B. Graph showing the % of acidic forms, as measured by IEX, on Palivizumab antibody in F45 formulation when compared with Synagis®-EU & Synagis®-US, when submitted to forced degradation conditions comprising high pH and low pH at 3 to 7 days, oxidation stress for 3, 7 and 14 days, agitation for 3 days and high temperature for 7 and 14 days.

FIG. 5A, FIG. 5B and FIG. 5C. Graph showing the % of intact antibody analyzed by CE-NR for Synagis®, palivizumab F43, F44, F45 and F48 formulations while maintaining the formulations at a temperature of 25° C. for up to 12 weeks (FIG. 5A); at 40° C. for up to 2 weeks (FIG. 5B) and at 2-8° C. for up to 1 year (FIG. 5C).

FIG. 6A, FIG. 6B and FIG. 6C. Graph showing the % of heavy chain (HC) and light chain (LC) of the antibody analyzed by CE-R for Synagis®, palivizumab F43, F44, F45 and F48 formulations while maintaining the formulations at a temperature of 25° C. for up to 12 weeks (FIG. 6A); at 40° C. for up to 2 weeks (FIG. 6B) and at 2-8° C. for up to 1 year (FIG. 6C).

FIG. 7A and FIG. 7B. (A) Graph showing the % of high-molecular weight species or levels of aggregates measured by SEC of Pembrolizumab antibody on F0 formulation compared with F24, F25 and F26 formulations when maintaining both formulations at a temperature of 40° C. for up to 2 weeks. (B) Graph showing the % of high-molecular weight species or levels of aggregates measured by SEC of Pembrolizumab antibody on F0 formulation compared with F24, F25 and F26 formulations when maintaining both formulations at a temperature of 25° C. for up to 12 weeks.

FIG. 8A, FIG. 8B and FIG. 8C. (A) Graph showing the % of acidic forms analyzed by IEX of Pembrolizumab antibody on F0 formulation compared to F24, F25 and F26 formulations when maintaining the formulations at a temperature of 40° C. for up to 2 weeks. (B) Graph showing the % of acidic forms of Pembrolizumab antibody on F0 formulation compared to F24, F25 and F26 formulations when maintaining both formulations at a temperature of 25° C. for up to 6 months. (C) Graph showing the % of acidic forms analyzed by IEX of Pembrolizumab antibody on F0 formulation compared to F24, F25 and F26 formulations when maintaining the formulations at a temperature between 2-8° C. for up to 6 months.

FIG. 9A, FIG. 9B and FIG. 9C. (A) Graph showing the % of basic forms analyzed by cIEF of Pembrolizumab antibody on F0 formulation compared to F24, F25 and F26 formulations when maintaining both formulations at a temperature of 40° C. for up to 2 weeks. (B) Graph showing the % of basic forms analyzed by cIEF of Pembrolizumab antibody on F0 formulation compared to F24, F25 and F26 formulations when maintaining both formulations at a temperature of 25° C. for up to 6 months. (C) Graph showing the % of basic forms analyzed by cIEF of Pembrolizumab antibody on F0 formulation compared to F24, F25 and F26 formulations when maintaining both formulations at a temperature between 2-8° C. for up to 6 months.

FIG. 10. Graph showing the positive or negative impact observed for the studied excipients in the % of high-molecular weight species or levels of aggregates (Agg) measured by SEC when maintaining the Pembrolizumab formulations showed in Table 3 at a temperature of 40° C. for up to 4 weeks (T40C 4w). Different buffers (Buf) (acetate and acetate-His), different sugars (Sug) (mannitol, trehalose and dextran 40) and antioxidant (Ant) (methionine) or no antioxidant were evaluated.

FIG. 11. Graph showing the positive or negative impact observed for the studied excipients in the % of acidic species (% Ac) analyzed by IEX when maintaining the Pembrolizumab formulations showed in Table 3 at a temperature of 40° C. for up to 4 weeks (T40C 4w). Different buffers (Buf) (acetate and acetate-His), different sugars (Sug) (mannitol, trehalose and dextran 40), amino acids (Amino) (arginine, proline or no amino acid) and antioxidants (Ant) (methionine) or no antioxidant were evaluated.

FIG. 12. Graph showing the confirmation data analysis of the % of high-molecular weight species or levels of aggregates (% Agg) by SEC at TO, after at least two of freeze/thaw cycles on the different studied excipients or combination of excipients showed in Pembrolizumab formulations of Table 4. Different buffers sugars (Sug) (mannitol and trehalose) and amino acids (Amino) (arginine and proline) or combinations thereof were evaluated.

FIG. 13A and FIG. 13B. (A) Graph showing the positive or negative impact observed for the studied excipients (Histidine-Acetate (HisAc), Trehalose (Tre), Arginine (Arg) in the % of high-molecular weight species or levels of aggregates measured by SEC rate (Δ% HMW) from time point 0 to time point 2 weeks (ΔT0 T2w) or time point 0 to time point 4 weeks (ΔT0 T4w). (B) Graph showing the positive or negative impact observed for the studied excipients in the % of acidic species (ACI) analyzed by IEX from time point 0 to time point 2 weeks (ΔT0 T2w) or time point 0 to time point 4 weeks (ΔT0 T4w).

FIG. 14 (A): Graph showing the % of high-molecular weight species or levels of aggregates measured by SEC rate (Δ% HMW) when maintaining the formulation showed in table 5 from time point 0 to time point 1 month, 3 months and 6 months (% ΔT0-T1m, T0-T3m, T0-T6m) at 25° C. (B) Graph showing the % of high-molecular weight species or levels of aggregates measured by SEC rate (Δ% HMW)) when maintaining the formulation showed in table 5 from time point 0 to time point 3 month, 6 months and 12 months (% ΔT0-T3m, T0-T6m, T0-T12m) at 2-8° C. Pembrolizumab biosimilar (MB12) formulated in F0 and in F45′, and Keytruda® reference product were tested.

FIG. 15 (A): Graph showing the % of acidic species rate (% ΔAc) analyzed by IEX when maintaining the Pembrolizumab formulations showed in Table 5 at a temperature of 40° C. for up to 4 weeks (ΔT0-T4w). (B) Graph showing the % of acidic species rate (% ΔAc) analyzed by IEX when maintaining the Pembrolizumab formulations showed in Table 5 at a temperature of 25° C. for 3 months and up to 6 months (ΔT0-T3m, ΔT0-T6m). Pembrolizumab biosimilar (MB12) formulated in F0 and in F45′, and Keytruda® reference product were tested

FIG. 16 (A): Graph showing the % of high-molecular weight species or levels of aggregates (% HMW) measured by SEC when incubating the Pembrolizumab formulations showed in Table 5 at a temperature of 25° C. for up to 3 days (3D) in Non-PVC (PO) bags. (B) Graph showing the % of high-molecular weight species or levels of aggregates rate (Δ% HMW) measured by SEC, from time point 0 to time point 3D (% ΔT0-3D) at 25° C. Pembrolizumab biosimilar (MB12) formulated in F0 and in F45′, and Keytruda® reference product were tested in non-PVC bags for in use stability.

FIG. 17 (A): Graph showing the % of high-molecular weight species or levels of aggregates (% HMW) measured by SEC when incubating the Pembrolizumab formulations showed in Table 5 at a temperature of 2-8° C. for up to 14 days (14D). (B) Graph showing the % of high-molecular weight species or levels of aggregates measured by SEC rate (Δ% HMW) from time point 0 to time point 14D (% ΔT0-14D) at 2-8° C. Pembrolizumab biosimilar (MB12) formulated in F0 and in F45′, and Keytruda® reference product were tested in non-PVC bags for in use stability.

FIG. 18 (A): Graph showing the number of subvisible particles/ml in the range of ≥10 μm % measured by Micro-Flow Imaging (MFI) when incubating the Pembrolizumab formulations showed in Table 5 at a temperature of 25° C. for up to 3 days (3D). (B) Graph showing the number of subvisible particles/ml in the range of ≥25 μm % measured by Micro-Flow Imaging (MFI) when incubating the Pembrolizumab formulations showed in Table 5 at a temperature of 25° C. for up to 3 days (3D). Pembrolizumab biosimilar (MB12) formulated in F0 and in F45′, and Keytruda® reference product were tested in non-PVC bags for in use stability.

FIG. 19 (A): Graph showing the % of high-molecular weight species or levels of aggregates measured by SEC rate (Δ% HMW) when incubating the Pembrolizumab formulations showed in Table 6 from time point 0 to time point 1 month (% ΔT0-T1m) at 40° C. (B) Graph showing the % of high-molecular weight species or levels of aggregates measured by SEC rate (Δ% HMW) when incubating the Pembrolizumab formulations showed in Table 6 from time point 0 to time point 1 month (% ΔT0-T1m) at 25° C. Pembrolizumab biosimilar (MB12) formulated in F0 and in F45′ at a concentration of 25 mg/ml, Keytruda® reference at 25 mg/ml and MB12 at a concentration of 165 mg/ml formulated in F48′ and F49′ were tested.

FIG. 20 (A): Graph showing the % of acidic species rate (% ΔAc) analyzed by IEX when maintaining the Pembrolizumab formulations showed in Table 6 at a temperature of 40° C. for up to 1 month (ΔT0-T1m). (B) Graph showing the % of acidic species rate (% ΔAc) analyzed by IEX when maintaining the Pembrolizumab formulations showed in Table 6 at a temperature of 25° C. for 1 months (ΔT0-T1m). Pembrolizumab biosimilar (MB12) formulated in F0 and in F45′ at a concentration of 25 mg/ml, Keytruda® reference at 25 mg/ml and MB12 at a concentration of 165 mg/ml formulated in F48′ and F49 were tested.

FIG. 21: Static light Scattering (SLS) at 266 nm was measured in a range of temperature from 25° C.-95° C. F48′ formulation (pembrolizumab at 165 mg/mL) was compared with the viscosity evolution of Keytruda® (25 mg/ml).

### DETAILED DESCRIPTION OF THE INVENTION

The present disclosure relates to the provision of formulations for stabilizing antibodies, in particular, for use in the prevention or treatment of diseases, as well as new methods for preparing stabilized antibody-containing compositions.

### 1. Formulation

The present disclosure relates to compositions that exhibit optimal characteristics for retaining or enhancing stability of antibodies, in particular, therapeutic antibodies.

The authors have developed formulations comprising an antibody, a buffer, an amino acid and trehalose, which are particularly useful in pH ranges between 5.0 to 7.0. The authors have found that the formulations of the disclosure provides long term stability at temperature ranges from 40° C. to −80° C. while retaining the biological activity of the antibody comprised in the formulation. In particular, the presence of trehalose is not only responsible for protecting the antibody molecule during freezing and thawing cycles, but also for providing enhanced stability during processes in which the antibody undergoes low pH conditions, for example, during protein purification or at stages of viral inactivation, required for pharmaceutical product safety. Therefore, the present disclosure provides effective means for simultaneously enhancing temperature-related antibody molecule stability and for reducing antibody aggregation at low pH conditions. Enhancement of long-term stability in a formulation according to the disclosure also results in lower production and treatment costs. The formulations according to the disclosure exhibits optimal properties for administration, storage and manipulation of antibodies, in particular, therapeutical antibodies. Manipulation includes, for example, lyophilization, reconstitution, dilution, titration and the like.

Thus, in a first aspect, the disclosure relates to formulations comprising an antibody, a buffer, an amino acid and trehalose, having a pH between 5.0 to 7.0.

Throughout this specification, the word “comprise” or variations such as “comprises” or “comprising” will be understood to imply the inclusion of a stated integer (or component) or group of integers (or components), but not the exclusion of any other integer (or component) or group of integers (or components).

Throughout the specification, where compositions are described as having, including, or comprising (or variations thereof), specific components, it is contemplated that compositions also may consist essentially of, or consist of, the recited components. Similarly, where methods or processes are described as having, including, or comprising specific process steps, the processes also may consist essentially of, or consist of, the recited processing steps. Further, it should be understood that the order of steps or order for performing certain actions is immaterial as long as the compositions and methods described herein remains operable.

As used herein, the term “about” modifying the quantity of an ingredient or component in the compositions of the disclosure refers to the variation in the numerical quantity that can occur, for example, through typical measuring and liquid handling procedures used for making pharmaceutical compositions in the real world; through inadvertent error in these procedures; through differences in the manufacture, source, or purity of the ingredients employed to make the compositions or carry out the methods; and the like without having a substantial effect on the chemical or physical attributes of the compositions of the disclosure. Such variation can be within an order of magnitude, typically within 10%, more typically still within 5%, of a given value or range. The term “about” also encompasses amounts that differ due to different equilibrium conditions for a composition resulting from a particular initial mixture. Whether or not modified by the term “about,” the paragraphs include equivalents to the quantities. Reference to “about” a value or parameter herein includes (and describes) embodiments that are directed to that value or parameter per se. For example, description referring to “about X” includes description of “X.” Numeric ranges are inclusive of the numbers defining the range.

The term “formulation” refers to a mixture of components and/or excipients that provides with long-term stability to one or more molecules comprised therein under a variety of conditions and applications. The term “formulation” also refers to effective means of delivering an efficacious and safe amount of a therapeutically active molecule. As used herein, the term “formulation” refers to a pharmaceutically acceptable medium that is compatible with an antibody, in particular, a therapeutical antibody, and it is safe and non-toxic when administered to a mammal, in particular, to humans.

According to the present disclosure, the therapeutically active molecule comprises at least one recombinant or natural therapeutical protein, in particular, at least one antibody.

As used herein, the term “antibody” or “Ab” refers to an immunoglobulin molecule (e.g., complete antibodies, antibody fragment or modified antibodies) capable of recognizing and binding to a specific target or antigen, such as a carbohydrate, polynucleotide, lipid, polypeptide, etc., through at least one antigen recognition site, located in the variable region of the immunoglobulin molecule. As used herein, the term “antibody” can encompass any type of antibody, including but not limited to monoclonal antibodies, polyclonal antibodies, human antibodies, engineered antibodies (including humanized antibodies, fully human antibodies, chimeric antibodies, multispecific antibodies, single-chain antibodies, artificially selected antibodies, CDR-granted antibodies, etc.) that specifically bind to a given antigen. In some embodiments, the antibody is a monoclonal antibody.

As used herein, the term “antibody” when used in reference to an antibody is also intended to mean a portion or functional fragment of an antibody which still retains some or all of its specific antigen binding activity.

As used herein, the term “functional fragment” when used in reference to an antibody is intended to mean a portion of an antibody which still retains some or all of its specific antigen binding activity. Such functional fragments can include, for example, antibody functional fragments such as Fd, Fv, Fab, F(ab′), F(ab)₂, F(ab′)₂, single chain Fv (scFv), chimeric antibodies, diabodies, triabodies, tetrabodies and minibody. Other functional fragments can include, for example, heavy (H) or light (L) chain polypeptides, variable heavy (VH) and variable light (VL) chain region polypeptides, complementarity determining region (CDR) polypeptides, single domain antibodies, and polypeptides that contain at least a portion of an immunoglobulin that is sufficient to retain its specific binding activity. With respect to antibodies and functional fragments thereof, various forms, alterations and modifications are well known in the art.

The term “monoclonal antibody” as used herein refers to an antibody obtained from a population of substantially homogeneous antibodies, i.e., the individual antibodies contained in the population may be present in small amounts. Except for naturally occurring mutations and/or post-translational modifications (e.g., isomerization, amidation) monoclonal antibodies are very specific and directed against a single antigenic site. Furthermore, in contrast to general (polyclonal) antibody preparations which typically include different antibodies to different determinants (epitopes), each monoclonal antibody is directed against a single determinant on the antigen. In addition to their specificity, monoclonal antibodies are advantageous in that they are synthesized by hybridoma culture without being contaminated by other immunoglobulins. The modifier “monoclonal” indicates the character of the antibody as obtained from a substantially homogeneous population of antibodies, and does not imply that the antibody has to be generated in any particular way. The term “monoclonal antibody” refers to an antibody that is derived from a single cell clone or hybridoma, including any eukaryotic, prokaryotic, or phage clone, and not the method by which it is produced. The monoclonal antibodies used in accordance with the disclosure can be made by the hybridoma method first described by Kohler et al., Nature, 256:495 (1975), or can be made by recombinant DNA methods known in the art.

The monoclonal antibodies of the disclosure can include any of such various monoclonal antibody forms, alterations and modifications. As used herein, an antibody can have one or more binding sites. If there is more than one binding site, the binding sites may be identical to one another or may be different. For example, a naturally occurring immunoglobulin has two identical binding sites, a single-chain antibody or Fab fragment has one binding site, while a “bispecific” or “bifunctional” antibody has two different binding sites.

In some embodiments, the term “antibody” is understood as a polypeptide product of B cells within the immunoglobulin class of polypeptides which is composed of heavy and light chains and able to bind with a specific molecular target or antigen. In some embodiments, “antibody” and/or “immunoglobulin” refers to a polypeptide comprising at least two heavy (H) chains (about 50-70 kDa) and two light (L) chains (about 25 kDa), optionally inter-connected by disulfide bonds.

The term “immunoglobulin” (lg) is used interchangeably herein with “antibody”. The term “immunoglobulin” includes five major classes of immunoglobulins: IgA, IgD, IgE, IgG and IgM, with heavy chains called α, δ, ε, γ and μ respectively. Furthermore, the classes of γ and α are divided into subclasses based on relatively minor differences such as CH sequence and function, for example, in humans the following subclasses are expressed: IgG1, IgG2, IgG3, IgG4, IgA1 and IgA2.

A “chimeric antibody” as used herein refers to an antibody that contains one or more regions from one antibody and one or more regions from one or more other antibodies.

A “humanized antibody” or “grafted antibody” as used herein has a sequence that differs from a non-human species antibody sequence by one or more amino acid substitutions, deletions, and/or additions, such that the humanized antibody is less likely to induce an immune response, and/or induces a less severe immune response, as compared to the non-human species antibody, when it is administered to a human subject. In one specific example, certain amino acids in the framework and constant domains of the heavy and/or light chains of the non-human species antibody are changed to produce the humanized antibody. In another specific example, the constant domain(s) from a human antibody are fused to the variable domain(s) of a non-human species.

A “human antibody” refers to antibodies that have one or more variable and constant regions derived from human immunoglobulin sequences. For example, a fully human antibody includes an antibody where all of the variable and constant domains are derived from human immunoglobulin sequences.

A “neutralizing antibody” (or an inhibitory antibody) when used in reference to a formulated antibody of the disclosure may refer to an antibody that inhibits the binding of receptor to ligand. Binding inhibition can occur by at least 10%, at least 20%, at least 30%, at least 40%, at least 50%, at least 60%, at least 70%, at least 75%, at least 80%, at least 85%, at least 90%, at least 95%, at least 97%, at least 99%, and at least 99.9%. The binding reduction may be measured by any means known to one of ordinary skill in the art, for example, as measured in an in vitro competitive binding assay.

As used herein, “biological activity” of an antibody refers to the ability of the antibody to bind to an antigen. It can further include antibody binding to an antigen and resulting in a measurable biological response which can be measured in vitro or in vivo. Such activity may be antagonistic or agonistic.

An “antagonistic” antibody refers to an antibody that inhibits an activity response of its antigen. Diminution in activity can be by at least 5%, at least 10%, at least 20%, at least 30%, at least 40%, at least 50%, at least 60%, at least 70%, at least 80%, at least 90% or 100%.

An “agonistic” antibody refers to an antibody that activates a response of its antigen. Increase in activity can be by at least 5%, at least 10%, at least 20%, at least 30%, at least 40%, at least 50%, at least 60%, at least 70%, at least 80%, at least 90% or 100%.

As used herein, the term “specific” when used in reference to an antibody, in particular monoclonal antibody binding activity, is intended to mean that the referenced monoclonal antibody exhibits preferential binding for its antigen compared to other similar antigens.

As used herein, “components” or “excipients” as comprised in the formulation of the disclosure are useful, for example, as a diluent, vehicle, buffer, stabilizer, tonicity agent, bulking agent, surfactant, cryoprotectant, lyoprotectant, anti-oxidant, metal ion source, chelating agent and/or preservative. As used herein, the term “component”, except for the term antibody, is intended to mean a therapeutically inactive substance.

The term “biosimilar” (also known as follow-on biologics) is well known in the art, and the skilled person would readily appreciate when a drug substance would be considered a biosimilar of an antibody. The term “biosimilar” is generally used to describe subsequent versions (generally from a different source) of “innovator biopharmaceutical products” (“biologics” whose drug substance is made by a living organism or derived from a living organism or through recombinant DNA or controlled gene expression methodologies) that have been previously officially granted marketing authorisation. Typically, biosimilars show a biological activity which is similar to that of the innovator biopharmaceutical product. Accordingly, as used herein, a biosimilar of the antibody used in the formulations disclosed herein is characterized in that it retains an activity of at least about 50%, 55%, 60%, 65%, 70%, 75%, 80%, 85%, 90%, 95% or 100% and can include activity measurements greater than 100% such as 105%, 110%, 115%, 120%, 125% or 150% or more compared to the activity of the innovator antibody. Suitable assays for determining the biological activity of an antibody are well-known in the art and can be used by the person skilled in the art depending on the specific antibody. By way of example, methods for determining the biological activity of palivizumab, pembrolizumab and nivolumab are described in the present application in the context of the determination of the stability of the antibody in the formulation and can be equally applied to determine whether a given antibody is a biosimilar of a reference antibody.

Stability of a formulation comprising an antibody, according to the disclosure, refers to the retention of structure, biological activity and/or function of the antibodies within a formulation. An antibody comprised in the formulation of the disclosure exhibit attributes such as resistance to change or deterioration that affect stability or function, and therefore consistently maintains functional characteristics over time. Accordingly, formulations of the disclosure exhibit, for example, reliability and safety with respect to activity per volume or activity units.

As used herein, the term “stable” or “stabilized” formulation refers to the physical stability and/or chemical stability and/or biological stability of a component, typically an active or composition thereof, during preservation/storage. Stability can be measured for a selected period of time at a selected temperature. For example, the degree of aggregation of the protein during storage can be used as an indicator of protein stability. Thus, a “stable” formulation may be one in which less than about 10%, preferably less than about 5% of the protein is present in the formulation as aggregates. Various analytical techniques for measuring protein stability are available in the art. A protein “retains its physical stability” in a pharmaceutical formulation if it shows no signs or very little of aggregation, precipitation and/or denaturation upon visual examination of color and/or clarity, or as measured by UV light scattering or by size exclusion chromatography.

A protein “retains its chemical stability” in a formulation according to the disclosure, if the chemical stability at a given time is such that the protein is considered to still retain its biological activity as defined below. Chemical stability can be assessed by detecting and quantifying chemically altered forms of the protein. Chemical alteration may involve size modification (e.g., clipping) which can be evaluated using size exclusion chromatography, SDS-PAGE and/or matrix-assisted laser desorption ionization/time-of-flight mass spectrometry (MALDIITOF MS), for example. Other types of chemical alteration include charge alteration (e.g., occurring as a result of deamidation) which can be evaluated by ion exchange chromatography or iciEF (image capillary isoelectric focusing), for example. Methods useful in the present disclosure to measure agitation-induced aggregation include gel electrophoresis, isoelectric focusing, capillary electrophoresis, chromatography such as size exclusion chromatography, ion exchange chromatography, reverse phase high performance liquid chromatography, peptide mapping, oligosaccharide mapping, mass spectrometry, ultraviolet absorption spectroscopy, fluorescence spectroscopy, circular dichroism spectroscopy, isothermal titration calorimetry, differential scanning calorimetry, analytical ultracentrifugation, dynamic light scattering, proteolysis, and crosslinking, detection of aggregation by turbidity measurement, filter retardation assay, immunological assay, fluorochrome binding assay, protein staining assay, microscopy, and ELISA or other binding assay.

An antibody “retains its biological activity” in a pharmaceutical formulation, if the biological activity of the antibody at a given time is within about 10% (within the errors of the assay) of the biological activity exhibited at the time the pharmaceutical formulation was prepared, for example, as determined in an antigen binding assay.

Stability can be measured at a selected temperature for a selected time period. In some embodiments, the formulation is stable at about 40° C. for at least about 1, 2, 3, 4, 5, 6, 7, 14, 21, 28, or more days. In certain embodiments, the formulation is stable at about 40° C. for at least about 1, 2, 3, 4, 5, 6, 7, 8, or more weeks.

In some embodiments, the formulation is stable at about 25° C. for at least about 1, 2, 3, 4, 5, 6, 7, 8, 9, 10, 11, 12, 13, 14, 15, 16, 17, 18, 19, 20, 21, 22, 23, 24, or more weeks. In some embodiments, the formulation is stable at about 25° C. for at least about 1, 2, 3, 4, 5, 6, 7, 8, 9, 10, 11, 12, 13, 14, 15, 16, 17, 18, 19, 20, 21, 22, 23, 24, or more months.

In certain embodiments, the formulation is stable at about 2-8° C. for at least about 1, 2, 3, 4, 5, 6, 7, 8, 9, 10, 11, 12, 13, 14, 15, 16, 17, 18, 19, 20, 21, 22, 23, 24, or more weeks. In certain embodiments, the formulation is stable at about 2-8° C. for at least about 1, 2, 3, 4, 5, 6, 7, 8, 9, 10, 11, 12, 13, 14, 15, 16, 17, 18, 19, 20, 21, 22, 23, 24, or more months.

In certain embodiments, the formulation is stable at about 5° C. for at least about 1, 2, 3, 4, 5, 6, 7, 8, 9, 10, 11, 12, 13, 14, 15, 16, 17, 18, 19, 20, 21, 22, 23, 24, or more weeks. In certain embodiments, the formulation is stable at about 5° C. for at least about 1, 2, 3, 4, 5, 6, 7, 8, 9, 10, 11, 12, 13, 14, 15, 16, 17, 18, 19, 20, 21, 22, 23, 24, or more months.

In some embodiments, the formulation is stable at about −20° C. for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or more weeks. In some embodiments, the formulation is stable at about −20° C. for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or more months.

In some embodiments, the formulation is stable at −80° C. for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or more weeks. In some embodiments, the formulation is stable at −80° C. for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or more months.

In some embodiments, the formulation is stable following freezing (to, e.g., −20° C., −40° C. or −80° C.) and thawing of the formulation, for example, following 1, 2 3, 4, or 5 cycles of freezing and thawing.

In some embodiments, the formulation is stable when submitted to forced degradation conditions for at least about 1, 2, 3, 4, 5, 6, 7, 8, 9, 10, 11, 12, 13, 14, 15, 16, 17, 18, 19, 20, 21, 22, 23, 24, 25, 26, 27, 28 days. Examples of forced degradation conditions include, but are not limited to, high pH (e.g., incubation at pH about 8-10 and about 2-8° C.), low pH (e.g., incubation at pH about 3-4.5 and about 2-8° C.), oxidation stress (e.g., addition of about 0.05% H₂O₂ and incubation at about 2-8° C.), agitation (e.g., stirring at 600 RPM at Room Temperature) and high temperature (e.g., incubation at about 40-60° C.).

Stability can be evaluated qualitatively and/or quantitatively in a variety of different ways, including, but not limited to, (i) evaluation of aggregate formation (for example using size exclusion chromatography, by measuring turbidity, and/or by visual inspection); (ii) by assessing charge heterogeneity using cation or anion exchange chromatography, capillary isoelectric focusing (cIEF), image capillary isoelectric focusing (iciEF) or capillary zone electrophoresis; (iii) amino-terminal or carboxy-terminal sequence analysis; (iv) mass spectrometric analysis; (v) SDS-PAGE analysis to compare reduced and intact antibody; (vi) peptide map (for example tryptic or LYS-C) analysis; (vii) evaluating biological activity or antigen binding function of the antibody; etc. Instability may involve any one or more of: aggregation, deamidation (e.g., Asn deamidation), oxidation (e.g. Met oxidation), isomerization (e.g. Asp isomerization), clipping/hydrolysis/fragmentation (e.g. hinge region fragmentation), succinimide formation, unpaired cysteine(s), N-terminal extension, C-terminal processing, glycosylation differences, etc. In some embodiments, stability of a protein, in particular, an antibody is assessed following the methods known in the art, for example, by measuring protein concentration (UV280), pH, osmolality, visual inspection, conductivity, micro differential scanning calorimetry (μDSC), size exclusion chromatography, SDS capillary gel electrophoresis (in non-reducing conditions or reducing conditions: CE-SDS NR or CE-SDS R), capillary isoelectric focusing (cIEF) or ion exchange chromatography (analysis of charge variant content and profile), or antigen-binding capacity (e.g. by ELISA).

According to the present disclosure, the stability of an antibody within a formulation of the disclosure includes, for example, the retention of physical and/or chemical stability and/or biological stability. Antibody stability can be assessed by, for example, determining whether the antibody has been subjected to a physical degradation and/or chemical degradation including chemical modification of its structure. Preservation of stability of an antibody in a formulation of the disclosure includes, for example, preservation of physical or chemical stability between about 50-100%, 60-100%, 70-100%, 80-100%, 85-99%, 90-98%, 92-96% or 94-95% compared to the stability of the antibody at an initial time point. Preservation in stability of an antibody in a formulation of the disclosure includes, for example, preservation of physical stability between about 50-100%, 60-100%, 70-100%, 80-100%, 85-99%, 90-98%, 92-96% or 94-95% compared to the stability of the antibody at an initial time point. Preservation in stability of an antibody in a formulation of the disclosure includes, for example, preservation of chemical stability between about 50-100%, 60-100%, 70-100%, 80-100%, 85-99%, 90-98%, 92-96% or 94-95% compared to the stability of the antibody at an initial time point. Preservation in stability of an antibody in a formulation of the disclosure includes, for example, preservation of physical and chemical stability between about 50-100%, 60-100%, 70-100%, 80-100%, 85-99%, 90-98%, 92-96% or 94-95% compared to the stability of the antibody at an initial time point.

Accordingly, stability of an antibody within a formulation of the disclosure may refer to the preservation of stability greater than 99.5%, at least about 99%, 98%, 97%, 96%, 95%, 94%, 93%, 92%, 91%, 90%, 89%, 88%, 87%, 86%, 85%, 84%, 83%, 82%, 81% or 80% compared to the stability of the polypeptide at an initial time point.

As used herein, stability of an antibody within the formulation of the disclosure includes, for example, preservation of biological activity. The “biological activity” of an antibody refers to the ability of the antibody to bind to its antigen, resulting in a biological response. Antibody activity can be assessed using, for example, an in vitro, in vivo and/or in situ assay indicative of the antibody's function. Preservation of biological stability of an antibody in a formulation of the disclosure includes, for example, preservation of activity between about 50-100% or more, depending on the variability of the assay. Preservation in biological stability of an antibody in a formulation of the disclosure includes, for example, preservation of biological activity between about 60-100%, 70-100%, 80-100%, 85-99%, 90-98%, 92-96% or 94-95% compared to the activity of the antibody at an initial time point.

In some embodiments, the biological activity of the antibody is measured by the specific binding of said antibody to its antigen and the binding is compared to the binding of the antibody at an initial time point. The determination of the biological activity of the antibody can be carried out using one of a variety of methods known to those skilled in the art. Examples of such methods are various enzyme-linked immunosorbent assay (ELISA) or radioimmunoassay methods.

For example, palivizumab biological activity can be determined by measuring the specific binding of palivizumab antibody to RSV Protein F. For example, RSV-infected cells (such as HEp-2 cells) are probed by the palivizumab antibody in the formulation of the disclosure and the specific binding of the antibody to protein F is determined and compared to the specific binding of the antibody at an initial time point. Another example of the determination of palivizumab binding to RSV protein F is performed by coating a plate with protein F, blocking the plate with a blocking agent, addition of the palivizumab samples and addition of a secondary antibody and substrate.

For example, the biological activity of pembrolizumab or nivolumab antibody can be measured by a method on the ability of pembrolizumab or nivolumab to bind in a dose-dependent manner its antigen PD-1, blocking the PD-1/PD-L1 binding in a competitive ELISA assay. For example, a competitive ELISA assay wherein a plastic plate is covered with a fixed amount of PD-1 and left to be coated. After incubating the plates with a blocking reagent, different concentrations of the primary antibody (for example pembrolizumab or nivolumab) mixed with a fixed amount of PD-L1 are added onto the plate and incubated. Afterwards, an anti-PD-L1 antibody conjugated with HRP (horseradish peroxidase) is added, joining to PD-L1. Finally, the formation of complexes is revealed by the addition of HRP substrate, and its subsequent stop with Sulfuric acid, generating a colorimetric product detectable at 450 nm. The intensity of the signal is inversely proportional to the amount of pembrolizumab or nivolumab associated with PD-1. Comparing the dose-response curves generated with the reference standard and the biosimilar samples, the relative binding potency of the biosimilar to PD-1 can be calculated. The biological activity (potency) of the samples can be expressed as the percentage of bioactivity of the sample against the reference standard.

Accordingly, biological stability of an antibody within the formulation of the disclosure includes retention of activity of at least about 50%, 55%, 60%, 65%, 70%, 75%, 80%, 85%, 90%, 95% or 100% and can include activity measurements greater than 100% such as 105%, 110%, 115%, 120%, 125% or 150% or more compared to the activity of the antibody at an initial time point. Generally, an initial time point is selected to be the time that an antibody is first prepared in a formulation of the disclosure or first examined for quality (i.e., meets release specifications). An initial time point also can include the time at which an antibody is reformulated in a formulation of the disclosure. The reformulation can be, for example, at a higher concentration, lower concentration or at the same concentration of an initial preparation.

In some embodiments, the formulations of the disclosure show an enhanced stability compared to the antibody reference medical product. In some embodiments, the stability is evaluated as % of aggregate formation (for example using size exclusion chromatography, by measuring turbidity, and/or by visual inspection) or by assessing charge heterogeneity using cation or anion exchange chromatography. In some embodiments, the formulation of the disclosure comprising palivizumab antibody shows an enhanced stability compared to Synagis®. In some embodiments, the formulation of the disclosure comprising pembrolizumab antibody shows an enhanced stability compared to Keytruda®. In some embodiments, the formulation of the disclosure comprising nivolumab antibody shows an enhanced stability compared to Opdivo®.

The term “buffer”, as used herein, refers to an aqueous solution comprising a mixture of a weak acid and its conjugate base intended for pH regulation of a formulation. A buffer is used herein to control the pH in a range that optimizes the therapeutic effect, especially when the stability is pH dependent. Buffering agents suitable for use with the present disclosure include both organic and inorganic acids and their salts. For example, citric acid, phosphoric acid, succinic acid, tartaric acid, fumaric acid, gluconic acid, oxalic acid, lactic acid, and/or acetic acid. Furthermore, the buffer may consist of histidine, acetate, histidine-acetate and/or trimethylamine salts.

Herein, a “buffer system” comprises one or more buffering agent(s) and/or an acid/base conjugate(s) thereof, and more suitably comprises one or more buffering agent(s) and an acid/base conjugate(s) thereof, and most suitably comprises one buffering agent only and an acid/base conjugate thereof. Unless stated otherwise, any concentrations stipulated herein in relation to a “buffer system” (i.e., a buffer concentration) suitably refers to the combined concentration of the buffering agent(s) and/or acid/base conjugate(s) thereof. In other words, concentrations stipulated herein in relation to a “buffer system” suitably refer to the combined concentration of all the relevant buffering species (i.e., the species in dynamic equilibrium with one another, e.g. citrate/citric acid). As such, a given concentration of a histidine buffer system generally relates to the combined concentration of histidine and the imidazolium form of histidine. However, in the case of histidine, such concentrations are usually straightforward to calculate by reference to the input quantities of histidine or a salt thereof. The overall pH of the composition comprising the relevant buffer system is generally a reflection of the equilibrium concentration of each of the relevant buffering species (i.e., the balance of buffering agent(s) to acid/base conjugate(s) thereof). As used herein, “buffer” refers to a “buffered solution” that resists changes in pH by the action of its acid-base conjugate components. The pH of a “buffered solution” will change only slightly upon addition of a small quantity of strong acid or base due to the “buffering effect” imparted by the “buffering agent”.

In some embodiments, the buffer of the disclosure has a pH in the range from about 5.0 to about 7.0. In some embodiments, the pH is in the range from about 5.0 to 6.0. In some embodiments, the pH is in the range from about 5.5 to about 7.0, for example from 5.6 to 6.9, 5.7 to 6.8, 5.8 to 6.7, 5.9 to 6.6, 5.9 to 6.5, 6.0, 6.0 to 6.4, or 6.1 to 6.3. In one embodiment, the buffer has a pH 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, or 7.0.

As used herein, the buffer maintains the pH of liquid formulations through the product shelf-life and maintain the pH of, i.e., lyophilized formulations during the lyophilization process and upon reconstitution, etc.

The term “having a pH between 5.0 to 7.0” refers to the range of pH of the formulation according to the disclosure so that the stability of the antibody is preserved.

In some embodiments, the formulation described herein has a pH about 5 to about 6, about 5.1 to about 5.9, about 5.2 to about 5.8, about 5.3 to about 5.7, about 5.4 to about 5.6.

In some embodiments, the formulation described herein has a pH about 5.5 to about 6.5, about 5.8 to about 6.8, about 5.8 to 6.2, about 5.9 to about 6.5, about 6.0 to about 6.5, about 6.0 to about 6.4, or about 6.0 to about 6.2. In some embodiments, the formulation has a pH about 5.6, about 5.8, about 5.9, about 6.0, about 6.2, about 6.4, about 6.5, about 6.8, or about 7.0, including every value in between these numbers.

In one embodiment, the pH of the formulation is pH 5 to 6.

In one embodiment, the pH of the formulation is pH 5.5-6.1.

In another embodiment, the pH of the formulation is pH 5.4-5.5.

As used herein, a “stabiliser” refers to a component which facilitates maintenance of the structural integrity of the biopharmaceutical drug, particularly during freezing and/or lyophilization and/or storage (especially when exposed to stress). This stabilising effect may arise for a variety of reasons, though typically such stabilisers may act as osmolytes which mitigate against protein denaturation. Examples of stabilisers are amino acids (i.e. free amino acids not part of a peptide or protein, e.g. glycine, arginine, histidine, aspartic acid, lysine).

Agents used as buffering agents, antioxidants or surfactants according to the disclosure, are excluded from the meaning of the term “stabilisers” as used herein, even if they may exhibit, i.a. stabilising activity.

The term “histidine” refers to the α-amino acid with chemical formula C₆H₉N₃O₂. As used herein, a “histidine buffer” is any buffer containing L-histidine in a concentration or range of concentration sufficient to maintain the pH of the formulation of the disclosure between 5.0 to 7.0 and to preserve the stability of the antibody comprised therein. Examples of a concentration sufficient to maintain the pH of the formulation includes concentrations in the range of about 5-50 mM, about 15-40 mM, about 15-30 mM, in the range of about 15-25 mM, in the range of about 20-25 mM. Examples of histidine buffers include histidine chloride, histidine hydrochloride, histidine acetate, histidine phosphate, and histidine sulphate.

The term “arginine” refers to the α-amino acid with chemical formula C₆H₁₄N₄O₂. As used herein, “arginine buffer” is any buffer containing arginine in a concentration or range of concentration sufficient to maintain the pH of the formulation of the disclosure between 5.0 to 7.0 and to preserve the stability of the antibody comprised therein.

The term “sugar” refers to an organic compound comprising only carbon, hydrogen, and oxygen, usually with a hydrogen oxygen atom ratio of 2:1 and the empirical formula Cm(H20) n. The term “sugar” includes mono-, di-, oligo- and polysaccharides. Examples of sugars include glucose, fructose, galactose, xylose, ribose, sucrose, mannose, lactose, maltose, trehalose, starch, and glycogen. Preferably, the sugar is a non-reducing sugar. Non-reducing sugars are sugars which are not able to act as a reducing agent, as they do not comprise a free aldehyde or ketone group.

In one embodiment, the sugar is trehalose. It is a disaccharide formed by a 1,1-glycosidic bond between a glucose and a fructose unit. The term “trehalose”, as used herein, refers to a non-reducing sugar. It is a disaccharide formed by a 1,1-glycosidic bond between two α-glucose units with chemical formula C₁₂H₂₂O₁₁ (CAS No.: 99-20-7 (anhydrous), 6138-23-4 (dihydrate)). In some embodiments, the dihydrate form of trehalose is used. The concentration of trehalose dihydrate in the liquid pharmaceutical composition of the present invention is from about 50 mM to about 250 mM, in some embodiments the concentration of trehalose dihydrate is from about 95 mM to about 200 mM, in certain embodiments the concentration of trehalose dihydrate is about 95 mM, about 100 mM or about 200 mM.

In some embodiments, trehalose is present in the formulation of the disclosure at a concentration of from about 5 mM to about 500 mM, from about 10 mM to about 400 mM, from about 15 mM to about 300 mM, from about 20 mM to about 200 mM or from about 25 mM to about 100 mM.

In one embodiment, trehalose is present in the formulation of the disclosure at a concentration of 50 to 250 mM.

In some embodiments, trehalose is present in the formulation of the disclosure at a concentration of about 50 mM to about 250 mM, about 55 mM to about 240 mM, about 60 mM to about 230 mM, about 65 mM to about 220 mM, about 70 mM to about 210 mM, about 75 mM to about 200 mM, about 80 mM to about 190 mM, about 81 mM to about 180 mM, about 82 mM to about 170 mM, about 83 mM to about 160 mM, about 84 mM to about 150 mM, about 85 mM to about 140 mM, about 86 mM to about 135 mM, about 87 mM to about 130 mM, about 88 mM to about 125 mM, about 89 mM to about 120 mM, about 90 mM to about 115 mM, about 91 mM to about 110 mM, about 92 mM to about 108 mM, about 93 mM to about 106 mM, about 94 mM to about 104 mM, or about 95 mM to about 102 mM.

In another embodiment, trehalose is present in the formulation of the disclosure at a concentration of about 100 mM to about 250 mM.

In some embodiments, trehalose is present in the formulation of the disclosure at a concentration of about 100 mM to about 250 mM, about 110 mM to about 240 mM, about 120 mM to about 230 mM, about 130 mM to about 220 mM, about 140 mM to about 210 mM, or about 150 mM to about 200 mM.

In some embodiments, trehalose is present in the formulation of the disclosure at a concentration of about 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM or 100 mM.

In one embodiment, trehalose is present in the formulation of the disclosure at a concentration of about 95 mM.

In another embodiment, trehalose is present in the formulation of the disclosure at a concentration of about 100 mM.

In some embodiments, trehalose is present in the formulation of the disclosure at a concentration of about 100 mM, 110 mM, 120 mM, 130 mM, 140 mM, 150 mM, 160 mM, 170 mM, 180 mM, 190 mM, 200 mM, 210 mM, 220 mM, 230 mM, 240 mM or 250 mM.

In one embodiment, trehalose is present in the formulation of the disclosure at a concentration of about 200 mM.

As used herein, “citrate buffer” is any buffer containing citrate in a concentration or range of concentration sufficient to maintain the pH of the formulation of the disclosure between 5.0 to 7.0 and to preserve the stability of the antibody comprised therein. The citrate buffer of the disclosure can include, for example, citric acid, citrate ion and/or citrate including citric acid salt forms. For example, “citrate buffer”, as used herein, is intended to refer to a buffer containing citric acid (chemical formula C₆H₈O₇) in equilibrium with its respective conjugate base.

As used herein, “acetate buffer” is any buffer containing acetate in a concentration or range of concentration sufficient to maintain the pH of the formulation of the disclosure between 5.0 to 7.0 and to preserve the stability of the antibody comprised therein. The acetate buffer of the disclosure can include, for example, acetic acid, acetate ion and/or acetate including acetic acid salt forms. For example, “acetate buffer”, as used herein, is intended to refer to a buffer containing acetic acid (chemical formula C₂H₄O₂) in equilibrium with its respective conjugate base.

In some embodiments, the buffer is selected from the group consisting of a histidine buffer, a citrate buffer, an acetate buffer, an arginine buffer or combinations thereof.

In one particular embodiment, the buffer in the formulation is a histidine buffer. In another embodiment, the histidine buffer is present in the formulation at a concentration of about 5 mM to about 50 mM histidine buffer.

In some embodiments, the histidine buffer is present in the formulation at a concentration of 1 mM to 500 mM, 2 mM to 400 mM, 3 mM to 300 mM, 4 mM to 200 mM, 5 mM to 100 mM, 6 mM to 90 mM, 7 mM to 80 mM, 8 mM to 70 mM, 9 mM to 60 mM, or 10 mM to 50 mM.

In some embodiments, the histidine buffer is present in the formulation at a concentration of 5 mM to 50 mM, 6 mM to 49 mM, 7 mM to 48 mM, 8 mM to 47 mM, 9 mM to 46 mM, 10 mM to 45 mM, 11 mM to 44 mM, 12 mM to 43 mM, 13 mM to 42 mM, 14 mM to 41 mM, 15 mM to 40 mM, 16 mM to 39 mM, 17 mM to 38 mM, 18 mM to 37 mM, 19 mM to 36 mM, 20 mM to 35 mM, 21 mM to 34 mM, 22 mM to 33 mM, 23 mM to 32 mM, 24 mM to 31 mM, or 25 mM to 30 mM.

In some embodiments, the histidine buffer is present in the formulation at a concentration of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM.

In one embodiment, the histidine buffer is present in the formulation at a concentration of about 25 mM histidine buffer. In another embodiment, the histidine buffer is present in the formulation at a concentration of about 10 mM histidine buffer. In another embodiment, the histidine buffer is present in the formulation at a concentration of about 5 mM histidine buffer

In one particular embodiment, the histidine buffer in the formulation according to the disclosure is a histidine-acetate buffer.

In another embodiment, the histidine-acetate buffer is present in the formulation at a concentration of about 5 mM to about 50 mM histidine buffer.

In some embodiments, in the histidine-acetate buffer, histidine is present at a concentration of 1.1 mM, 1.2 mM, 1.3 mM, 1.4 mM, 1.5 mM, 1.6 mM, 1.7 mM, 1.8 mM, 1.9 mM, 2 mM, 2.1 mM, 2.2 mM, 2.3 mM, 2.4 mM, 2.5 mM, 2.6 mM, 2.7 mM, 2.8 mM, 2.9 mM, 3 mM, 4 mM, 5 mM, 6 mM, 7 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, or 25 mM, and acetate is present at a concentration of 1.1 mM, 1.2 mM, 1.3 mM, 1.4 mM, 1.5 mM, 1.6 mM, 1.7 mM, 1.8 mM, 1.9 mM, 2 mM, 2.1 mM, 2.2 mM, 2.3 mM, 2.4 mM, 2.5 mM, 2.6 mM, 2.7 mM, 2.8 mM, 2.9 mM, 3 mM, 4 mM, 5 mM, 6 mM, 7 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, or 25 mM.

In some embodiments, the histidine-acetate buffer is present in the formulation at a concentration of 1 mM to 200 mM, 2 mM to 150 mM, 3 mM to 100 mM, 5 mM to 50 mM, 6 mM to 49 mM, 7 mM to 48 mM, 8 mM to 47 mM, 9 mM to 46 mM, 10 mM to 45 mM, 11 mM to 44 mM, 12 mM to 43 mM, 13 mM to 42 mM, 14 mM to 41 mM, 15 mM to 40 mM, 16 mM to 39 mM, 17 mM to 38 mM, 18 mM to 37 mM, 19 mM to 36 mM, 20 mM to 35 mM, 21 mM to 34 mM, 22 mM to 33 mM, 23 mM to 32 mM, 24 mM to 31 mM, or 25 mM to 30 mM.

In some embodiments, the histidine-acetate buffer is present in the formulation at a concentration of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, or 25 mM.

In one embodiment, the histidine-acetate buffer is present in the formulation of the disclosure at a concentration of about 5 mM histidine-acetate buffer. In another embodiment, the histidine-acetate buffer is present in the formulation of the disclosure at a concentration of about 10 mM histidine-acetate buffer. In another embodiment, the histidine-acetate buffer is present in the formulation of the disclosure at a concentration of about 25 mM histidine-acetate buffer.

The term “amino acid” as used herein refers to any organic compounds that contains an amino group —NH₂ and a carboxyl group-COOH functional groups, along with a side chain (R group) specific to each amino acid.

Examples of suitable amino acids according to the present disclosure include alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, or combinations thereof. In some embodiments, the amino acid is glycine. In other embodiments, the amino acid is arginine. In other embodiments, the amino acid is proline.

In some embodiments, the amino acid present in the formulation of the disclosure is selected from the group consisting of glycine, histidine, lysine, arginine, proline, serine, methionine and alanine.

In some embodiments, the amino acid is present in the formulation at a concentration of about 1 mM to about 500 mM, about 2 mM to about 400 mM, about 3 mM to about 300 mM, about 4 mM to about 200 mM, about 5 mM to about 190 mM, about 6 mM to about 180 mM, about 7 mM to about 175 mM, about 8 mM to about 170 mM, about 9 mM to about 165 mM, about 10 mM to about 160 mM, about 11 mM to about 155 mM, about 12 mM to about 150 mM about 13 mM to about 145 mM, about 14 mM to about 140 mM, or about 15 mM to about 135 mM.

In one embodiment, the amino acid is present in the formulation at a concentration of about 1-155 mM.

In some embodiments, the amino acid present in the formulation of the disclosure is arginine or proline.

In some embodiments, arginine is present in the formulation at a concentration of about 5 mM to about 200 mM, about 6 mM to about 195 mM, about 7 mM to about 190 mM, about 8 mM to about 185 mM, about 9 mM to about 180 mM, about 10 mM to about 175 mM, about 11 mM to about 170 mM, about 12 mM to about 165 mM about 13 mM to about 160 mM, about 14 mM to about 155 mM, or about 15 mM to about 150 mM.

In one embodiment, arginine is present in the formulation at a concentration of about 5-155 mM.

In one embodiment, arginine is present in the formulation at a concentration of about 155 mM.

In other embodiment, arginine is present in the formulation at a concentration of about 110 mM.

In some embodiments, proline is present in the formulation at a concentration of about 5 mM to about 200 mM, about 6 mM to about 195 mM, about 7 mM to about 190 mM, about 8 mM to about 185 mM, about 9 mM to about 180 mM, about 10 mM to about 175 mM, about 11 mM to about 170 mM, about 12 mM to about 165 mM about 13 mM to about 160 mM, about 14 mM to about 155 mM, or about 15 mM to about 150 mM.

In one embodiment, proline is present in the formulation at a concentration of about 155 mM.

In another embodiment, proline is present in the formulation at a concentration of about 110 mM.

In another embodiment, the amino acid present in the formulation of the invention disclosure is glycine.

In some embodiments, glycine is present in the formulation of the disclosure at a concentration of 1 mM to 3 mM. In some embodiments, glycine is in the formulation of the disclosure at a concentration of about 1 mM to about 3 mM, about 1.1 mM to about 2.9 mM, about 1.2 mM to about 2.8 mM, about 1.3 mM to about 2.7 mM, about 1.4 mM to about 2.6 mM, or about 1.5 mM to about 2.5 mM,

In one embodiment, glycine is present in the formulation of the disclosure at a concentration of about 1.6 mM.

As used herein, the term “surfactant” refers to a substance that functions to reduce the surface tension of a liquid in which it is dissolved. Surfactants can be included in a formulation for a variety of purposes including, for example, to prevent or control aggregation, particle formation and/or surface adsorption in liquid formulations or to prevent or control these phenomena during the lyophilization and/or reconstitution process in lyophilized formulations. Surfactants include, for example, amphipathic organic compounds that exhibit partial solubility in both organic solvents and aqueous solutions. General characteristics of surfactants include their ability to reduce the surface tension of water, reduce the interfacial tension between oil and water and also form micelles. Surfactants of the disclosure include non-ionic and ionic surfactants. Surfactants are well known in the art. Examples of surfactants that may be used include polysorbate (for example, polysorbate 20 (polyoxyethylene (20) sorbitan monolaurate, also known under the tradename Tween® 20); polysorbate 80 (polyoxyethylene (20) sorbitan monooleate, also known under the tradename Tween® 80); poloxamer (e.g. poloxamer 188, a non-ionic triblock copolymers composed of a central hydrophobic chain of polyoxypropylene (poly(propylene oxide)) flanked by two hydrophilic chains of polyoxyethylene (poly(ethylene oxide)), also known under the tradename Lutrol® F 68).

Agents used as buffering agents, antioxidants or stabilisers according to the disclosure, are excluded from the meaning of the term “surfactants” as used herein, even if they may exhibit, i.e., surfactant activity.

In some embodiments, the formulation of the disclosure comprises a surfactant. In some embodiments, the surfactant is a non-ionic surfactant. The use of non-ionic surfactants in the formulation of the disclosure include, for example, alkyl poly (ethylene oxide), alkyl polyglucosides such as octyl glucoside and decyl maltoside, fatty alcohols such as cetyl alcohol and oleyl alcohol, cocamide MEA, cocamide DEA, and cocamide TEA. Specific examples of non-ionic surfactants include the polysorbates including, for example, polysorbate 20, polysorbate 28, polysorbate 40, polysorbate 60, polysorbate 65, polysorbate 80, polysorbate 81, polysorbate 85 and the like; the poloxamers including, for example, poloxamer 188, also known as poloxalkol or poly(ethylene oxide)-poly(propylene oxide), poloxamer 407 or polyethylene-polypropylene glycol and the like, and polyethylene glycol (PEG). Polysorbate 20 is synonymous with TWEEN® 20, sorbitan monolaurate and polyoxyethylenesorbitan monolaurate. The present disclosure contemplates the use of any surfactant with average molecular weight from 7680 to 9510 g/mol.

In one embodiment, the formulation of the disclosure further comprises at least one surfactant.

In some embodiments, the surfactant is present in the formulation of the disclosure at a concentration is of about 0.01 to about 0.2%, about 0.02 to about 0.18%, about 0.04 to about 0.16%, about 0.06 to about 0.18%, about 0.08 to about 0.14%.

In another embodiment, the surfactant is present in the formulation of the disclosure at a concentration is of 0.01-0.2%.

In one embodiment, the surfactant is present in the formulation of the disclosure at about 0.2%.

In some embodiments, the surfactant present in the formulation of the disclosure is selected from the group consisting of polysorbate 80, polysorbate 20, poloxamer, poloxamer 188, and combinations thereof. In some embodiments, the surfactant is selected from the group consisting of polysorbate 80, polysorbate 20, poloxamer, poloxamer 188 and a surfactant with average molecular weight from 7680 to 9510 g/mol.

The term “poloxamer” as used herein refers to non-ionic triblock copolymers composed of a central hydrophobic chain of polyoxypropylene (poly(propylene oxide)) flanked by two hydrophilic chains of polyoxyethylene (poly(ethylene oxide)). As used herein, the term “poloxamer 188” (also poly(ethylene glycol)-block-poly(propylene glycol)-block-poly(ethylene glycol) refers to the poloxamer with formula (C₃H₆O·C₂H₄O)ₓ and CAS No.: 9003-11-6.

In some embodiments, poloxamer 188 is present in the formulation of the disclosure at a concentration is of about 0.01 to about 0.2%, about 0.02 to about 0.18%, about 0.04 to about 0.16%, about 0.06 to about 0.18%, about 0.08 to about 0.14%.

In another embodiment, the surfactant present in the formulation of the disclosure is poloxamer 188 and it is found at a concentration of between 0.01 and 0.2% (w/V).

In one embodiment, poloxamer 188 is present in the formulation of the disclosure at a concentration of about 0.2% (w/V).

In some embodiments, the formulation of the disclosure comprises an antibody at a concentration of about 1 mg/ml to 500 mg/ml, about 2 mg/ml to 400 mg/ml, about 3 mg/ml to 300 mg/ml, about 4 mg/ml to 200 mg/ml, about 5 mg/ml to 190 mg/ml, about 6 mg/ml to 180 mg/ml, about 7 mg/ml to 170 mg/ml, about 8 mg/ml to 160 mg/ml, about 9 mg/ml to 150 mg/ml, about 10 mg/ml to 140 mg/ml, about 11 mg/ml to 130 mg/ml, about 12 mg/ml to 120 mg/ml, about 13 mg/ml to 110 mg/ml, or about 14 mg/ml to 100 mg/ml.

In one embodiment, the formulation of the disclosure comprises an antibody at a concentration of about 10 mg/ml to about 100 mg/ml.

In some embodiments, the antibody comprised in the formulation is at a concentration of about 10 mg/mL to about 100 mg/mL, about 15 mg/mL to about 95 mg/mL, about 20 mg/mL to about 90 mg/mL, about 25 mg/mL to about 85 mg/mL, about 30 mg/mL to about 80 mg/mL, about 40 mg/mL to about 75 mg/mL, about 45 mg/mL to about 70 mg/mL, or about 50 mg/mL to about 65 mg/mL.

In some embodiments, the antibody comprised in the formulation is at a concentration of about 25 mg/mL, about 30 mg/mL, about 35 mg/mL, about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, or about 100 mg/mL.

In one embodiment, the antibody comprised in the formulation is at a concentration of about 100 mg/ml.

In some embodiments, the antibody comprised in the formulation is at a concentration of about 1 mg/mL, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml, about 11 mg/ml, about 12 mg/ml, about 13 mg/ml, about 14 mg/ml, about 15 mg/ml, about 16 mg/ml, about 17 mg/ml, about 18 mg/ml, about 19 mg/ml, about 20 mg/ml, about 21 mg/ml, about 22 mg/ml, about 23 mg/ml, about 24 mg/ml, or about 25 mg/ml.

In one embodiment, the antibody comprised in the formulation is at a concentration of about 25 mg/ml.

In another embodiment, the antibody comprised in the formulation is at a concentration of about 10 mg/ml.

In one embodiment, the antibody comprised in the formulation is an immunoglobulin IgG1.

In another embodiment, the antibody comprised in the formulation is an immunoglobulin IgG4.

In a further embodiment, the antibody comprised in the formulation is an immunoglobulin IgG1 or immunoglobulin IgG4.

As used herein, the term “Palivizumab” refers to a humanized monoclonal antibody (IgG1k) composed of two heavy chains (50.6 kDa each) and two light chains (27.6 kDa each), which contains 1-2% carbohydrate by weight and which is directed against an epitope in the A antigenic site of the F protein of RSV. The reference medical product (RMP) of palivizumab is Synagis®. It is presented in lyophilized powder and in solution format (0.5 and 1 ml at 100 mg/ml) for intramuscular injection. Binding of the antibody to its cognate antigen on the RSV surface renders the viral particle no longer infectious or pathogenic. In some embodiments, the antibody included within the formulation is a biosimilar antibody of Synagis®.

SEQ ID NO:1 shows the full-length heavy chain amino acid sequence of palivizumab. SEQ ID NO:2 shows the full-length light chain amino acid sequence of palivizumab. SEQ ID NO:3 shows the amino acid sequence of the variable region of the heavy chain sequence of palivizumab. SEQ ID NO:4 shows the amino acid sequence of the variable region of the light chain sequence of palivizumab. SEQ ID NO:5 shows the amino acid sequence of the CDR1 of the variable region of the heavy chain sequence of palivizumab. SEQ ID NO:6 shows the amino acid sequence of the CDR2 of the variable region of the heavy chain sequence of palivizumab. SEQ ID NO:7 shows the amino acid sequence of the CDR3 of the variable region of the heavy chain sequence of palivizumab. SEQ ID NO:8 shows the amino acid sequence of the CDR1 of the variable region of the light chain sequence of palivizumab. SEQ ID NO:9 shows the amino acid sequence of the CDR2 of the variable region of the light chain sequence of palivizumab. SEQ ID NO:10 shows the amino acid sequence of the CDR3 of the variable region of the light chain sequence of palivizumab.

In one embodiment, the antibody comprised in the formulation of the disclosure is a neutralizing antibody against the Respiratory Syncytial Virus (RSV).

In another embodiment, the neutralizing antibody comprised in the formulation of the disclosure is Palivizumab or a biosimilar thereof.

In some embodiments, Palivizumab is present in the formulation of the disclosure at a concentration of 5 mg/ml to 500 mg/ml, 10 mg/ml to 400 mg/ml, 20 mg/ml to 300 mg/ml, 30 mg/ml to 200 mg/ml, 40 mg/ml to 150 mg/ml, or 50 mg/ml to 100 mg/ml.

In some embodiments, Palivizumab is present in the formulation of the disclosure at a concentration of 10 mg/ml, 20 mg/ml, 30 mg/ml, 40 mg/ml, 50 mg/ml, 40 mg/ml, 50 mg/ml, 60 mg/ml, 70 mg/ml, 80 mg/ml, 90 mg/ml, 100 mg/ml, 110 mg/ml, 120 mg/ml, 130 mg/ml, 140 mg/ml, 150 mg/ml, 160 mg/ml, 170 mg/ml, 180 mg/ml, 190 mg/ml, or 200 mg/ml.

In one embodiment, Palivizumab is present in the formulation of the disclosure at a concentration of about 100 mg/ml.

In some embodiments, Palivizumab is present in the formulation of the disclosure at a concentration of about 1 mg/mL, 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml, 21 mg/ml, 22 mg/ml, 23 mg/ml, 24 mg/ml, or 25 mg/ml.

In some embodiments, the amino acid present in the formulation is glycine at a concentration of about 0.2 mM to about 10 mM, about 0.4 mM to about 9 mM, about 0.6 mM to about 8 mM, about 0.8 mM to about 7 mM, about 1 mM to about 6 mM, about 1.2 mM to about 5 mM, about 1.4 mM to about 4 mM, about 1.6 mM to about 3 mM, about 1.8 mM to about 2.8 mM, about 2 mM to about 2.6 mM, or about 2.2 mM to about 2.4 mM; the buffer present in the formulation is histidine buffer at a concentration of about 5 mM to 50 mM, 6 mM to 49 mM, 7 mM to 48 mM, 8 mM to 47 mM, 9 mM to 46 mM, 10 mM to 45 mM, 11 mM to 44 mM, 12 mM to 43 mM, 13 mM to 42 mM, 14 mM to 41 mM, 15 mM to 40 mM, 16 mM to 39 mM, 17 mM to 38 mM, 18 mM to 37 mM, 19 mM to 36 mM, 20 mM to 35 mM, 21 mM to 34 mM, 22 mM to 33 mM, 23 mM to 32 mM, 24 mM to 31 mM, or 25 mM to 30 mM; and trehalose is present in the formulation at a concentration of about 10 mM to about 250 mM, about 15 mM to about 220 mM, about 20 mM to about 210 mM, about 30 mM to about 200 mM, about 40 mM to about 190 mM, about 50 mM to about 180 mM, about 60 mM to about 170 mM, about 70 mM to about 160 mM, about 75 mM to about 150 mM, about 80 mM to about 140 mM, about 85 mM to about 130 mM, or about 90 mM to about 120 mM.

In one embodiment, in the formulation of the disclosure the amino acid is glycine at a concentration of 1.6 mM, the buffer is histidine buffer at a concentration of 25 mM and trehalose is present at a concentration of 100 mM.

In another embodiment, in the formulation of the disclosure, the amino acid is glycine at a concentration of 1.6 mM, the buffer is histidine buffer at a concentration of 25 mM, trehalose is present at a concentration of 100 mM and the antibody is palivizumab or a biosimilar thereof.

In another embodiment, in the formulation of the disclosure, the amino acid is glycine at a concentration of 1.6 mM, the buffer is histidine buffer at a concentration of 25 mM, trehalose is present at a concentration of 100 mM and the antibody is pembrolizumab or a biosimilar thereof.

In another embodiment, in the formulation of the disclosure, the amino acid is glycine at a concentration of 1.6 mM, the buffer is histidine buffer at a concentration of 25 mM, trehalose is present at a concentration of 100 mM and the antibody is nivolumab or a biosimilar thereof.

The terms “anti-PD1 antibody” or “anti-PD-1 neutralizing antibody” refer to an antibody that specifically binds to cell death protein 1 (PD-1) and inhibits the binding of PD-1 to its ligand PD-L1 and optionally inhibits the binding of PD-1 to its ligands PD-L1 and PD-L2. The anti-PD1 antibody thereby abolishes the suppressive effect of the PD-1/PD-L1 interaction on T cells. Known anti-PD1 antibodies include, but are not limited to, pembrolizumab, nivolumab, cemiplimab and cetrelimab. In some embodiments, the anti-PD1 antibody is pembrolizumab or a biosimilar thereof. In some embodiments, the anti-PD1 antibody is nivolumab or a biosimilar thereof.

As used herein, the term “Pembrolizumab” (Keytruda®, also known as MK-3475, SCH 900475 and lambrolizumab) refers to a humanized (IgG4/kappa) antibody with the heavy chain mutation S228P (IgG4-Pro) that specifically binds the programmed cell death protein 1 (PD-1) and blocks interaction of this protein with its ligands PD-1 ligand (PD-L1) and PD-1 ligand 2 (PD-L2). Pembrolizumab is described, for example, in U.S. Pat. Nos. 8,354,509 and 8,900,587; see also http://www.cancer.gov/drugdictionary?cdrid=695789 (last accessed: Dec. 14, 2014) and comprises the heavy and light chain amino acid sequences and CDRs described in Table 2 of WO 2018/204368.

The present commercial pembrolizumab formulation (Keytruda®) contains 10 mM histidine, 70 mg/ml sucrose, 0.2 mg/ml polysorbate 80 and water for injection, pH 5.5 and is supplied in a concentration of 25 mg/ml. In some embodiments, the antibody included within the formulation of the disclosure is a biosimilar antibody of Keytruda®.

SEQ ID NO:11 shows the full-length heavy chain amino acid sequence of pembrolizumab. SEQ ID NO: 12 shows the full-length light chain amino acid sequence of pembrolizumab. SEQ ID NO:13 shows the amino acid sequence of the variable region of the heavy chain sequence of pembrolizumab. SEQ ID NO:14 shows the amino acid sequence of the variable region of the light chain sequence of pembrolizumab. SEQ ID NO: 15 shows the amino acid sequence of the CDR1 of the variable region of the heavy chain sequence of pembrolizumab. SEQ ID NO:16 shows the amino acid sequence of the CDR2 of the variable region of the heavy chain sequence of pembrolizumab. SEQ ID NO: 17 shows the amino acid sequence of the CDR3 of the variable region of the heavy chain sequence of pembrolizumab. SEQ ID NO:18 shows the amino acid sequence of the CDR1 of the variable region of the light chain sequence of pembrolizumab. SEQ ID NO:19 shows the amino acid sequence of the CDR2 of the variable region of the light chain sequence of pembrolizumab. SEQ ID NO:20 shows the amino acid sequence of the CDR3 of the variable region of the light chain sequence of pembrolizumab.

As used herein, the term “Nivolumab” (also known as “OPDIVO®”; formerly designated 5C4, BMS-936558, MDX-1106, or ONO-4538) is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol Res. 2 (9): 846-56). Nivolumab is approved for the treatment of recurrent non-small cell lung cancer, melanoma, and renal cell carcinoma, among others. It comprises the heavy and light chain amino acid sequences and CDRs described in Table 2 of WO 2018/204368.

The present commercial nivolumab formulation contains 30 mg/ml mannitol, 0.008 mg/ml pentetic acid, 0.2 mg/ml polysorbate 80, 2.92 mg/ml sodium chloride, 5.88 mg/ml sodium citrate dihydrate, and water for injection, pH 6.0, and is supplied in a concentration of 10 mg/ml. In some embodiments, the antibody included within the formulation of the disclosure is a biosimilar antibody of Opdivo®.

SEQ ID NO:21 shows the full-length heavy chain amino acid sequence of nivolumab. SEQ ID NO:22 shows the full-length light chain amino acid sequence of nivolumab. SEQ ID NO:23 shows the amino acid sequence of the variable region of the heavy chain sequence of nivolumab. SEQ ID NO:24 shows the amino acid sequence of the variable region of the light chain sequence of nivolumab. SEQ ID NO:25 shows the amino acid sequence of the CDR1 of the variable region of the heavy chain sequence of nivolumab. SEQ ID NO:26 shows the amino acid sequence of the CDR2 of the variable region of the heavy chain sequence of nivolumab. SEQ ID NO:27 shows the amino acid sequence of the CDR3 of the variable region of the heavy chain sequence of nivolumab. SEQ ID NO:28 shows the amino acid sequence of the CDR1 of the variable region of the light chain sequence of nivolumab. SEQ ID NO:29 shows the amino acid sequence of the CDR2 of the variable region of the light chain sequence of nivolumab. SEQ ID NO: 30 shows the amino acid sequence of the CDR3 of the variable region of the light chain sequence of nivolumab.

In another embodiment, the antibody comprised in the formulation of the disclosure is an IgG4.

In one embodiment, the antibody comprised in the formulation of the disclosure is an anti-PD-1 neutralizing antibody.

In another embodiment, the antibody comprised in the formulation of the disclosure is Pembrolizumab.

In some embodiments, Pembrolizumab is present in the formulation of the disclosure at a concentration of about 1 mg/mL, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml, about 11 mg/ml, about 12 mg/ml, about 13 mg/ml, about 14 mg/ml, about 15 mg/ml, about 16 mg/ml, about 17 mg/ml, about 18 mg/ml, about 19 mg/ml, about 20 mg/ml, about 21 mg/ml, about 22 mg/ml, about 23 mg/ml, about 24 mg/ml, or about 25 mg/ml.

In some embodiments, Pembrolizumab is present in the formulation of the disclosure at a concentration of about 10 mg/ml, about 20 mg/ml, about 30 mg/ml, about 40 mg/ml, about 50 mg/ml, about 40 mg/ml, about 50 mg/ml, about 60 mg/ml, about 70 mg/ml, about 80 mg/ml, about 90 mg/ml, about 100 mg/ml, about 110 mg/ml, about 120 mg/ml, about 130 mg/ml, about 140 mg/ml, or about 150 mg/ml.

In one embodiment, Pembrolizumab is present in the formulation of the disclosure at concentration of about 25 mg/ml.

In another embodiment, the antibody comprised in the formulation of the disclosure is Nivolumab.

In some embodiments, Nivolumab is present in the formulation of the disclosure at a concentration of about 1 mg/mL, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml, about 11 mg/ml, about 12 mg/ml, about 13 mg/ml, about 14 mg/ml, about 15 mg/ml, about 16 mg/ml, about 17 mg/ml, about 18 mg/ml, about 19 mg/ml, about 20 mg/ml, about 21 mg/ml, about 22 mg/ml, about 23 mg/ml, about 24 mg/ml, or about 25 mg/ml.

In some embodiments, Nivolumab is present in the formulation of the disclosure at a concentration of about 10 mg/ml, about 20 mg/ml, about 30 mg/ml, about 40 mg/ml, about 50 mg/ml, about 40 mg/ml, about 50 mg/ml, about 60 mg/ml, about 70 mg/ml, about 80 mg/ml, about 90 mg/ml, or about 100 mg/ml.

In one embodiment, Nivolumab is present in the formulation of the disclosure at a concentration of about 10 mg/ml.

In some embodiments, the amino acid present in the formulation is arginine at a concentration of about 40 mM to about 200 mM, about 45 mM to about 190 mM, about 50 mM to about 185 mM, about 55 mM to about 180 mM, about 60 mM to about 175 mM, about 65 mM to about 170 mM, about 70 mM to about 165 mM, about 75 mM to about 160 mM, about 80 mM to about 155 mM, about 98 mM to about 150 mM, about 11 mM to about 140 mM, about 12 mM to about 135 mM about 13 mM to about 130 mM, about 14 mM to about 125 mM, or about 15 mM to about 120 mM; the buffer present in the formulation is histidine-acetate buffer at a concentration of about a 1 mM, 2 mM, 3 mM, 4 mM, 5 mM, 6 mM, 7 mM, 8 mM, 9 mM, or 10 mM; and trehalose is present in the formulation at a concentration of about 50 mM to about 300 mM, about 60 mM to about 280 mM, about 70 mM to about 260 mM, about 80 mM to about 240 mM, about 100 mM to about 220 mM, about 120 mM to about 210 mM, about 130 mM to about 200 mM, about 140 mM to about 190 mM, or about 150 mM to about 180 mM.

In one embodiment, in the formulation of the disclosure the amino acid is arginine at a concentration of 80-155 mM, the buffer is a 5 mM histidine-acetate buffer and trehalose is at a concentration of 100-250 mM.

In one embodiment, in the formulation of the disclosure the amino acid is arginine at a concentration of 80-155 mM, the buffer is a 5 mM histidine-acetate buffer, trehalose is at a concentration of 100-250 mM and the antibody is pembrolizumab or a biosimilar thereof.

In one embodiment, in the formulation of the disclosure the amino acid is arginine at a concentration of 80-155 mM, the buffer is a 5 mM histidine-acetate buffer, trehalose is at a concentration of 100-250 mM and the antibody is nivolumab or a biosimilar thereof.

In some embodiments, the amino acid present in the formulation is arginine at a concentration of about 80 mM to about 155 mM, about 82 mM to about 150 mM, about 84 mM to about 145 mM, about 86 mM to about 140 mM, about 88 mM to about 135 mM, about 90 mM to about 130 mM, about 92 mM to about 125 mM, about 94 mM to about 120 mM, about 96 mM to about 115 mM, about 98 mM to about 110 mM, about 11 mM to about 105 mM, about 12 mM to about 100 mM about 13 mM to about 95 mM, about 14 mM to about 90 mM, or about 15 mM to about 85 mM; the buffer present in the formulation is histidine-acetate buffer at a concentration of about a 1 mM, 2 mM, 3 mM, 4 mM, 5 mM, 6 mM, 7 mM, 8 mM, 9 mM, or 10 mM; poloxamer at 0.01% w/v, 0.02% w/V, 0.05% w/v, 0.1% w/v, 0.12% w/v, 0.14% w/v, 0.16% w/v, 0.18, % w/v, 0.2% w/v 0.22% w/v, 0.23% w/v 0.24% w/v, 0.25% w/v, 0.26% w/v, 0.27% w/v, 0.28% w/v, 0.29% w/V, or 0.3% w/v; and trehalose is present in the formulation at a concentration of about 100 mM to about 250 mM, about 105 mM to about 240 mM, about 110 mM to about 230 mM, about 115 mM to about 220 mM, about 120 mM to about 210 mM, about 125 mM to about 200 mM, about 130 mM to about 190 mM, or about 140 mM to about 180 mM.

In some embodiments, the amino acid present in the formulation is arginine at a concentration of about 5 mM to about 155 mM, 10 mM to about 155 mM, 20 mM to about 155 mM, 40 mM to about 155 mM, 80 mM to about 155 mM, about 82 mM to about 150 mM, about 84 mM to about 145 mM, about 86 mM to about 140 mM, about 88 mM to about 135 mM, about 90 mM to about 130 mM, about 92 mM to about 125 mM, about 94 mM to about 120 mM, about 96 mM to about 115 mM, about 98 mM to about 110 mM, about 11 mM to about 105 mM, about 12 mM to about 100 mM about 13 mM to about 95 mM, about 14 mM to about 90 mM, about 15 mM to about 85 mM or about 5 mM to about 50 mM; the buffer present in the formulation is histidine-acetate buffer at a concentration of about 5 mM to about 50 mM, or about 10 to about 40 mM; poloxamer at 0.01% w/v, 0.02% w/v, 0.05% w/v, 0.1% w/v, 0.12% w/v, 0.14% w/v, 0.16% w/v, 0.18, % w/v, 0.2% w/v 0.22% w/v, 0.23% w/v 0.24% w/v, 0.25% w/v, 0.26% w/v, 0.27% w/V, 0.28% w/v, 0.29% w/v, or 0.3% w/v; and trehalose is present in the formulation at a concentration of about 50 mM to about 250 mM, about 100 mM to about 250 mM, about 105 mM to about 240 mM, about 110 mM to about 230 mM, about 115 mM to about 220 mM, about 120 mM to about 210 mM, about 125 mM to about 200 mM, about 130 mM to about 190 mM, or about 140 mM to about 180 mM.

In one embodiment, in the formulation of the disclosure the amino acid is arginine at a concentration of 110 mM, the buffer is a 5 mM histidine-acetate buffer, poloxamer is present at 0.2% w/v and trehalose is present at a concentration of 200 mM.

In one embodiment, in the formulation of the disclosure the amino acid is arginine at a concentration of 5-150 mM, the buffer is a 5-50 mM histidine-acetate buffer, poloxamer is present at 0.01-0.2% w/v and trehalose is present at a concentration of 50-250 mM.

In one embodiment, in the formulation of the disclosure the amino acid is arginine at a concentration of 110 mM, the buffer is a 5 mM histidine-acetate buffer, poloxamer is present at 0.2% w/v, trehalose is present at a concentration of 200 mM and the antibody is pembrolizumab or a biosimilar thereof.

In one embodiment, in the formulation of the disclosure the amino acid is arginine at a concentration of 5-150 mM, the buffer is a 5-50 mM histidine-acetate buffer, poloxamer is present at 0.01-0.2% w/v, trehalose is present at a concentration of 50-250 mM and the antibody is nivolumab or a biosimilar thereof.

In some embodiments, the amino acid present in the formulation is arginine at a concentration of about 80 mM to about 200 mM, about 82 mM to about 190 mM, about 84 mM to about 185 mM, about 86 mM to about 180 mM, about 88 mM to about 175 mM, about 90 mM to about 170 mM, about 92 mM to about 165 mM, about 94 mM to about 160 mM, about 96 mM to about 155 mM, about 98 mM to about 150 mM, about 11 mM to about 140 mM, about 12 mM to about 135 mM about 13 mM to about 130 mM, about 14 mM to about 125 mM, or about 15 mM to about 120 mM; the buffer present in the formulation is histidine-acetate buffer at a concentration of about a 1 mM, 2 mM, 3 mM, 4 mM, 5 mM, 6 mM, 7 mM, 8 mM, 9 mM, or 10 mM; poloxamer is present at 0.01% w/v, 0.02% w/v, 0.05% w/v, 0.1% w/v, 0.12% w/v, 0.14% w/v, 0.16% w/v, 0.18, % w/V, 0.2% w/v 0.22% w/v, 0.23% w/v 0.24% w/v, 0.25% w/v, 0.26% w/v, 0.27% w/v, 0.28% w/v, 0.29% w/v, or 0.3% w/v; and trehalose is present in the formulation at a concentration of about 10 mM to about 250 mM, about 15 mM to about 220 mM, about 20 mM to about 210 mM, about 30 mM to about 200 mM, about 40 mM to about 190 mM, about 50 mM to about 180 mM, about 60 mM to about 170 mM, about 70 mM to about 160 mM, 75 mM to about 150 mM, 80 mM to about 140 mM, 85 mM to about 130 mM, or 90 mM to about 120 mM.

In one embodiment, in the formulation of the disclosure the amino acid is arginine at a concentration of 155 mM, the buffer is a 5 mM histidine-acetate buffer, poloxamer is present at 0.2% w/v and trehalose is present at a concentration of 95 mM.

In one embodiment, in the formulation of the disclosure the amino acid is arginine at a concentration of 155 mM, the buffer is a 5 mM histidine-acetate buffer, poloxamer is present at 0.2% w/v, trehalose is present at a concentration of 95 mM and the antibody is pembrolizumab or a biosimilar thereof.

In one embodiment, in the formulation of the disclosure the amino acid is arginine at a concentration of 155 mM, the buffer is a 5 mM histidine-acetate buffer, poloxamer is present at 0.2% w/v, trehalose is present at a concentration of 95 mM and the antibody is nivolumab or a biosimilar thereof.

In some embodiments, the amino acid present in the formulation is proline at a concentration of 80 mM to about 155 mM, about 82 mM to about 150 mM, about 84 mM to about 145 mM, about 86 mM to about 140 mM, about 88 mM to about 135 mM, about 90 mM to about 130 mM, about 92 mM to about 125 mM, about 94 mM to about 120 mM, about 96 mM to about 115 mM, about 98 mM to about 110 mM, about 11 mM to about 105 mM, about 12 mM to about 100 mM about 13 mM to about 95 mM, about 14 mM to about 90 mM, or about 15 mM to about 85 mM; the buffer present in the formulation is histidine-acetate buffer at a concentration of about a 1 mM, 2 mM, 3 mM, 4 mM, 5 mM, 6 mM, 7 mM, 8 mM, 9 mM, or 10 mM; poloxamer is present at 0.01% w/V, 0.02% w/v, 0.05% w/v, 0.1% w/v, 0.12% w/v, 0.14% w/v, 0.16% w/v, 0.18, % w/v, 0.2% w/v 0.22% w/v, 0.23% w/v 0.24% w/v, 0.25% w/v, 0.26% w/v, 0.27% w/v, 0.28% w/V, 0.29% w/v, or 0.3% w/v; and trehalose is present in the formulation at a concentration of about 100 mM to about 250 mM, about 105 mM to about 240 mM, about 110 mM to about 230 mM, about 115 mM to about 220 mM, about 120 mM to about 210 mM, about 125 mM to about 200 mM, about 130 mM to about 190 mM, or about 140 mM to about 180 mM.

In one embodiment, in the formulation of the disclosure the amino acid is proline at a concentration of 110 mM, the buffer is a 5 mM histidine-acetate buffer, poloxamer is present at 0.2% w/v and trehalose is present at a concentration of 200 mM.

In one embodiment, in the formulation of the disclosure the amino acid is proline at a concentration of 5-155 mM, the buffer is a 5-50 mM histidine-acetate buffer, poloxamer is present at 0.01-0.2% w/v and trehalose is present at a concentration of 50-250 mM.

In one embodiment, in the formulation of the disclosure the amino acid is proline at a concentration of 110 mM, the buffer is a 5 mM histidine-acetate buffer, poloxamer is present at 0.2% w/v, trehalose is present at a concentration of 200 mM and the antibody is pembrolizumab or a biosimilar thereof.

In one embodiment, in the formulation of the disclosure the amino acid is proline at a concentration of 5-155 mM, the buffer is a 5-50 mM histidine-acetate buffer, poloxamer is present at 0.01-0.2% w/v, trehalose is present at a concentration of 50-250 mM and the antibody is pembrolizumab or a biosimilar thereof.

In one embodiment, in the formulation of the disclosure the amino acid is proline at a concentration of 110 mM, the buffer is a 5 mM histidine-acetate buffer, poloxamer is present at 0.2% w/v, trehalose is present at a concentration of 200 mM and the antibody is nivolumab or a biosimilar thereof.

In one embodiment, in the formulation of the disclosure the amino acid is proline at a concentration of 5-155 mM, the buffer is a 5-50 mM histidine-acetate buffer, poloxamer is present at 0.01-0.2% w/v, trehalose is present at a concentration of 50-250 mM and the antibody is nivolumab or a biosimilar thereof.

In some embodiments, the amino acid present in the formulation is proline at a concentration of about 80 mM to about 200 mM, about 82 mM to about 190 mM, about 84 mM to about 185 mM, about 86 mM to about 180 mM, about 88 mM to about 175 mM, about 90 mM to about 170 mM, about 92 mM to about 165 mM, about 94 mM to about 160 mM, about 96 mM to about 155 mM, about 98 mM to about 150 mM, about 11 mM to about 140 mM, about 12 mM to about 135 mM about 13 mM to about 130 mM, about 14 mM to about 125 mM, or about 15 mM to about 120 mM; the buffer present in the formulation is histidine-acetate buffer at a concentration of about a 1 mM, 2 mM, 3 mM, 4 mM, 5 mM, 6 mM, 7 mM, 8 mM, 9 mM, or 10 mM; poloxamer at 0.01% w/v, 0.02% w/v, 0.05% w/v, 0.1% w/v, 0.12% w/v, 0.14% w/v, 0.16% w/v, 0.18, % w/v, 0.2% w/V 0.22% w/v, 0.23% w/v 0.24% w/v, 0.25% w/v, 0.26% w/v, 0.27% w/v, 0.28% w/v, 0.29% w/v, or 0.3% w/v; and trehalose is present in the formulation at a concentration of about 10 mM to about 250 mM, about 15 mM to about 220 mM, about 20 mM to about 210 mM, about 30 mM to about 200 mM, about 40 mM to about 190 mM, about 50 mM to about 180 mM, about 60 mM to about 170 mM, about 70 mM to about 160 mM, 75 mM to about 150 mM, 80 mM to about 140 mM, 85 mM to about 130 mM, or 90 mM to about 120 mM.

In one embodiment, in the formulation of the disclosure the amino acid is proline at a concentration of 155 mM, the buffer is a 5 mM histidine-acetate buffer, poloxamer is present at 0.2% w/v and trehalose is present at a concentration of 95 mM.

In one embodiment, in the formulation of the disclosure the amino acid is proline at a concentration of 155 mM, the buffer is a 5 mM histidine-acetate buffer, poloxamer is present at 0.2% w/v, trehalose is present at a concentration of 95 mM and the antibody is pembrolizumab or a biosimlar thereof.

In one embodiment, in the formulation of the disclosure the amino acid is proline at a concentration of 155 mM, the buffer is a 5 mM histidine-acetate buffer, poloxamer is present at 0.2% w/v, trehalose is present at a concentration of 95 mM and the antibody is nivolumab or a biosimlar thereof.

In some embodiments, the amino acid present in the formulation is glycine at a concentration of about 0.2 mM to about 10 mM, about 0.4 mM to about 9 mM, about 0.6 mM to about 8 mM, about 0.8 mM to about 7 mM, about 1 mM to about 6 mM, about 1.2 mM to about 5 mM, about 1.4 mM to about 4 mM, about 1.6 mM to about 3 mM, about 1.8 mM to about 2.8 mM, about 2 mM to about 2.6 mM, or about 2.2 mM to about 2.4 mM; the buffer present in the formulation is histidine buffer at a concentration of about a at a concentration of 5 mM to 50 mM, 6 mM to 49 mM, 7 mM to 48 mM, 8 mM to 47 mM, 9 mM to 46 mM, 10 mM to 45 mM, 11 mM to 44 mM, 12 mM to 43 mM, 13 mM to 42 mM, 14 mM to 41 mM, 15 mM to 40 mM, 16 mM to 39 mM, 17 mM to 38 mM, 18 mM to 37 mM, 19 mM to 36 mM, 20 mM to 35 mM, 21 mM to 34 mM, 22 mM to 33 mM, 23 mM to 32 mM, 24 mM to 31 mM, or 25 mM to 30 mM; and trehalose is present in the formulation at a concentration of about 10 mM to about 250 mM, about 15 mM to about 220 mM, about 20 mM to about 210 mM, about 30 mM to about 200 mM, about 40 mM to about 190 mM, about 50 mM to about 180 mM, about 60 mM to about 170 mM, about 70 mM to about 160 mM, 75 mM to about 150 mM, 80 mM to about 140 mM, 85 mM to about 130 mM, or 90 mM to about 120 mM.

In one embodiment, the formulation does not contain sucrose.

In another embodiment, the formulation does not contain polysorbate 80.

In a further embodiment, the formulation does not contain sucrose and/or does not contain polysorbate 80.

In a further embodiment, the formulation does not contain mannitol.

In a further embodiment, the formulation does not contain pentetic acid.

In a further embodiment, the formulation does not contain sodium chloride or/and sodium citrate.

In a further embodiment, the formulation does not contain more than one antibody.

The antibody-containing formulation of the disclosure is particularly advantageous during the viral inactivation or elimination step of the antibody purification process. The viral inactivation usually takes place at very low pH values, for example, at pH 3-4.5. The formulation of the disclosure strongly reduces the low pH-associated degradation of the antibody. By using the formulation of the disclosure in the viral inactivation step, the degree of aggregation of the formulated antibody is notably reduced compared to a different formulation. For example, by formulating palivizumab antibody in the formulation of the disclosure, the level of antibody aggregates was significantly reduced compared to palivizumab reference product Synagis®. In some embodiments, the aggregates reduction is of about 1%, about 2%, about 3%, about Δ%, about 5%, about 6%, about 7%, about 8%, about 9% or about 10%, compared to the palivizumab reference product Synagis®.

### 2. Medical Uses

In a further aspect, the formulation of the disclosure is for use in the prevention and/or treatment of a disease caused by an infection by RSV.

In another aspect, the present disclosure provides a method for the prevention and/or treatment of a disease caused by an infection by RSV in a subject in need thereof, the method comprising administering to the subject a therapeutically effective amount of the formulation of the disclosure.

As used herein, the term “therapeutically effective amount” refers to that amount of the therapeutic agent being administered, as a single agent or in combination with one or more additional agents, which will relieve to some extent one or more of the symptoms of the condition being treated. In some embodiments, the therapeutically effective amount is an amount sufficient to effect the beneficial or desired clinical results. With respect to the treatment of cancer, a therapeutically effective amount refers to that amount which has at least one of the following effects: palliate, ameliorate, stabilize, reverse, prevent, slow or delay the progression of (and/or symptoms associated with) of cancer. The effective amounts that may be used in the present disclosure varies depending upon the manner of administration, the age, body weight, and general health of the subject. The appropriate amount and dosage regimen can be determined using routine skill in the art.

“Treating” or “treatment” of a state, disorder, disease or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

“Prevention” or “treatment” as used herein refers to both therapeutic treatment and prophylactic or preventative measures. Those in need of treatment include those already with the disorder as well as those in which the disorder is to be prevented. A “disorder” is any condition that would benefit from treatment including, but not limited to, chronic and acute disorders or diseases including those pathological conditions which predispose the mammal to the disorder in question.

The term “RSV” refers to the Respiratory Syncytial Virus (also human respiratory syncytial virus (hRSV) and human orthopneumovirus) which causes infections of the lungs and respiratory tract, usually exhibiting mild cold-like signs and symptoms. The RSV may, however, cause pneumonia or bronchiolitis if it spreads to the lower respiratory tract.

In a further aspect, the formulation of the disclosure is for use in the prevention and/or treatment of cancer.

In another aspect, the present disclosure provides a method for the prevention and/or treatment of cancer in a subject in need thereof, the method comprising administering to the subject a therapeutically effective amount of the formulation of the disclosure.

The terms “cancer”, “tumor”, “cell proliferative disorder” and “proliferative disorder” are used interchangeably, and they refer to disorders that are associated with some degree of abnormal cell proliferation. In one embodiment, the cell proliferative disorder is cancer. In one embodiment, the cell proliferative disorder is a tumor. “Tumor,” as used herein, refers to all neoplastic cell growth and proliferation, whether malignant or benign, and all pre-cancerous and cancerous cells and tissues. The terms “cancer”, “cancerous”, “cell proliferative disorder”, “proliferative disorder” and “tumor” are not mutually exclusive as referred to herein. The terms “cancer” and “cancerous” refer to or describe the physiological condition in mammals that is typically characterized by unregulated cell growth. Examples of cancer include, but are not limited to, prostate cancer, lung cancer, ovarian cancer, liver cancer, bladder cancer, hepatoma, thyroid cancer, renal cancer, esophageal carcinoma, head and neck carcinoma, Hodgkin and Non-Hodgkin lymphoma, primary mediastinal large B-cell lymphoma, colorectal carcinoma, gastric or gastroesophageal junction adenocarcinoma, cervical cancer, breast cancer, endometrial carcinoma, blastoma, sarcoma, merkel cell carcinomas, cutaneous squamous cell carcinoma, kidney cancer high mutational burden (TMB) cancers, microsatellite instability-high (MSI-H/dMMR) cancers and leukemia or lymphoid malignancies.

In one embodiment, the cancer is characterized in that it shows increased levels of PD-L1.

In another aspect, the formulation according to the present disclosure, when used in the treatment of cancer, may be co-administered with one or more therapeutic agents, e.g., a cytotoxic agent, a radiotoxic agent or an immunosuppressive agent. The antibody forming part of the formulation can be linked to the agent (as an immunocomplex) or can be administered separate from the agent. In the latter case (separate administration), the antibody can be administered before, after or concurrently with the agent or can be co-administered with other known therapies, e.g., an anti-cancer therapy, e.g., radiation. A cytotoxic agent includes any agent that is detrimental to (e.g., kills) cells.

Cytotoxic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclinies (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids). Further examples of cytotoxic agent to be used in combination with the formulations disclosed herein include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

Other preferred examples of therapeutic cytotoxins that can be co-administered with the formulations disclosed herein include duocarmycins, calicheamicins, maytansines and auristatins, and derivatives thereof.

In another embodiment, the formulation of the disclosure is for use in the prevention and/or treatment of a disease caused by increased levels of PD-L1.

Disease other than cancer which are characterized by increased levels of PD-L1 include, for example, infectious diseases in which the infectious agent acts by up-regulating PD-1 or PD-L1. Examples of pathogens for which this therapeutic approach may be particularly useful, include pathogens for which there is currently no effective vaccine, or pathogens for which conventional vaccines are less than completely effective. These include, but are not limited to HIV, Hepatitis (A, B, & C), Influenza, Herpes, Giardia, Malaria, Leishmania, Staphylococcus aureus, Pseudomonas Aeruginosa. PD-1 blockade is particularly useful against established infections by agents such as HIV that present altered antigens over the course of the infections.

Methods to determine if the levels of PD-L1 are increased in a subject are known in the art. For example, the immunohistochemistry assays are routinely used. The immunohistochemistry assays stain the tumor tissue specimens according to the presence or absence of PD-L1. Further, the level of expression can be quantified. For example, the kit PD-L1 IHC 22C₃ pharmDx® (Manufactured by Dako North America), an immunochemistry kit for assessing expression levels of PD-L1 has been approved by the FDA in relation to the use of Keytruda® for the treatment of Non-Small Cell Lung Cancer (NSCLC), gastric or gastroesophageal junction adenocarcinoma, cervical cancer, urothelial carcinoma, head and neck squamous cell carcinoma (HNSCC), esophageal squamous cell carcinoma and triple negative breast cancer (TNBC) can be used. Also, the kit PD-L1 IHC 28-8 pharmDx® (Manufactured by Dako North America) approved by the FDA in relation to the use of Opdivo® for Non-small cell lung cancer (NSCLC) in combination with Yervoy (Ipilimumab) may be used.

In one embodiment, the cancer is characterized in that it shows increased levels of mutations in solid tumors or microsatellite instability.

Methods to determine if the levels of mutations in solid tumors of a patient of cancer are known in the art. For example, methods based on the study of individual genomic profile of each patient's cancer. For example, next generation sequencing oncology panel, somatic or germline variant detection system in vitro diagnosis can be used. For example, the FoundationOne CDx (Foundation Medicine) approved by the FDA for the use of Keytruda in solid tumors may be used.

The route of administration of therapeutical proteins is well-known in the art, for example, single or multiple boluses or long-term infusion in a suitable manner, such as subcutaneous, intravenous, intraperitoneal, intramuscular, intraarterial, intralesional or intraarticular route. Also, topical administration, inhalation or sustained release or sustained release means, and the like are contemplated by the present disclosure. The methods of the disclosure may be combined with known methods of treatment for diseases as a combination or additional treatment step, or as an additional component of a therapeutic formulation.

The dosage and desired drug concentration of the pharmaceutical composition of the present disclosure will vary depending on the particular use envisioned. Determination of the appropriate dosage or route of administration is within the skill of the ordinary artisan.

The formulations of the disclosure, including but not limited to reconstituted formulations, are intravenously administered to mammals, preferably humans, in need of treatment with proteins, i.e., by bolus or continuous infusion over a period of time. The administration is carried out according to known methods such as intramuscular, intraperitoneal, intracerebrospinal, subcutaneous, intraarticular, intrasynovial, intrathecal, oral, topical or inhalation routes.

### 3. Method for Preparing a Stabilized Antibody-Containing Composition

The authors have found that formulating an antibody in a composition comprising a buffer, an amino acid and trehalose, results in enhanced stability of the antibody comprised therein when the formulation has a pH of 5.0 to 7.0.

Thus, in a further aspect, the disclosure relates to a method for preparing a stabilized antibody-containing composition comprising the step of formulating the antibody in a composition comprising a buffer, an amino acid and trehalose, and wherein the formulation has a pH of 5.0 to 7.0.

Methods of preparing antibodies and other proteins that can be formulated as described herein are well known in the art.

In another embodiment, the antibody is formulated in a first step together with the buffer and the amino acid, and in a second step trehalose is formulated together with the formulation obtained in the first step. The last step is to adjust the pH to the desired pH of 5.0-7.0, so as to obtain the composition according to the method of the disclosure.

The method for preparing a stabilized antibody-containing composition of the disclosure is particularly advantageous during the viral inactivation or elimination step of the antibody purification process. The viral inactivation usually takes place at very low pH values, for example, at pH 3-4.5. The preparation of the stabilized antibody-containing composition of the disclosure strongly reduces the low pH-associated degradation of the antibody. By formulating the composition of the disclosure according to the present method, the degree of aggregation of the formulated antibody is notably reduced compared to a different formulation. For example, by formulating palivizumab antibody by the method of the disclosure, the level of antibody aggregates was significantly reduced compared to palivizumab reference product Synagis®. In some embodiments, the aggregates reduction is of about 1%, about 2%, about 3%, about Δ%, about 5%, about 6%, about 7%, about 8%, about 9% or about 10%, compared to the palivizumab reference product Synagis®.

In some embodiments, the formulations described herein can be prepared as reconstituted lyophilized formulations. The proteins or antibodies described herein may be lyophilized and then reconstituted to produce a liquid formulation according to the disclosure. In a particular embodiment, after preparation of any protein of interest described above, a “pre-lyophilized formulation” may be produced. The amount of protein present in the pre-lyophilized formulation may be determined taking into consideration the desired dose, method of administration, and the like. A “reconstituted” formulation is one which is prepared by dissolving the lyophilized protein or antibody formulation in a diluent and dispersing the protein in a reconstituted formulation. The reconstituted formulation may be suitable for administration (e.g., parenteral administration) to a patient treated with the protein of interest.

In another embodiment, the formulation according to the method of the disclosure is prepared in liquid state and stored in liquid form, whereas storing at freezing temperatures result in freezing of the liquid formulation.

The term “stable” or “stabilized” antibody-containing composition as used herein refers to a composition in which the antibody therein essentially retains its physical stability and/or chemical stability and/or biological activity upon storage. Accordingly, a stable composition essentially retains its physical and chemical stability, as well as its biological activity upon storage. The storage period is generally selected based on the intended shelf-life of the formulation.

In one embodiment, the buffer formulated according to the method of the disclosure is selected from the group consisting of a histidine buffer, a citrate buffer, an acetate buffer, an arginine buffer or combinations thereof.

In another embodiment, the buffer is a histidine buffer.

In some embodiments, the histidine buffer formulated in the method of the disclosure contains 5 to 50 mM histidine.

In another embodiment, the histidine buffer formulated in the method of the disclosure contains 5 mM histidine.

In another embodiment, the histidine buffer formulated in the method of the disclosure contains 25 mM histidine.

In another embodiment, the buffer formulated in the method of the disclosure is a histidine-acetate buffer.

In some embodiments, the buffer formulated in the method of the disclosure contains histidine-acetate at a concentration from 5 mM to 50 mM.

In another embodiment, the histidine-acetate formulated in the method of the disclosure contains about 5 mM histidine-acetate buffer.

In another embodiment, the amino acid formulated in the method of the disclosure is selected from the group consisting of glycine, histidine, lysine, arginine, proline, serine, methionine and alanine.

In another embodiment, the amino acid is arginine or proline.

In some embodiments, arginine or proline are formulated in the method of the disclosure at a concentration of about 5-155 mM.

In one embodiment, arginine or proline is formulated at a concentration of about 155 mM.

In another embodiment, arginine or proline is formulated at a concentration of about 110 mM.

In another embodiment, the amino acid formulated in the method of the disclosure is glycine.

In some embodiments, glycine is formulated at a concentration of 1 to 3 mM.

In one embodiment, the glycine is formulated at a concentration of about 1.6 mM.

In some embodiments, the trehalose is formulated according to the method of the disclosure at a concentration of 50 to 250 mM.

In some embodiments, trehalose is formulated according to the method of the disclosure at a concentration of about 95 mM.

In some embodiments, trehalose is formulated at a concentration of 100 to 250 mM.

In some embodiments, trehalose is formulated according to at a concentration of about 200 mM or about 100 mM. In another embodiment, trehalose is formulated according to the present method at a concentration of 100 mM. In another embodiment, trehalose is formulated according to the present method at a concentration of 200 mM.

In one embodiment, the pH of the formulation according to the method of the disclosure is of about 5 to about 6.

In another embodiment, the pH of the formulation according to the method of the disclosure is of 5.4-5.5.

In another embodiment, the method comprises formulating an antibody in a composition further comprising a surfactant.

In another embodiment, the surfactant concentration is of 0.01-0.2%.

In one embodiment, the surfactant is formulated in the method at a concentration of about 0.2%.

In another embodiment, the formulated surfactant is poloxamer.

In another embodiment, the poloxamer is formulated according to the method at a concentration of between 0.01 and 0.2% (w/V).

In another embodiment, the poloxamer is at a concentration of about 0.2% (w/V).

In another embodiment, the antibody is formulated according to method of the disclosure at a concentration of 10-100 mg/ml.

In one embodiment, the antibody is formulated according to method of the disclosure at a concentration of 100 mg/ml.

In some embodiments, the antibody is formulated at a concentration of 25 mg/ml.

In some embodiments, the antibody is formulated at a concentration of 10 mg/ml.

In another embodiment, the formulated antibody is an IgG1 or an IgG4.

In another embodiment, the antibody formulated according to method of the disclosure is a neutralizing antibody against RSV.

In another embodiment, the formulated neutralizing antibody against RSV is Palivizumab or a biosimilar thereof.

In some embodiments, Palivizumab is formulated according to method of the disclosure at a concentration of 10 mg/ml, 20 mg/ml, 30 mg/ml, 40 mg/ml, 50 mg/ml, 40 mg/ml, 50 mg/ml, 60 mg/ml, 70 mg/ml, 80 mg/ml, 90 mg/ml, 100 mg/ml, 110 mg/ml, 120 mg/ml, 130 mg/ml, 140 mg/ml, 150 mg/ml, 160 mg/ml, 170 mg/ml, 180 mg/ml, 190 mg/ml, or 200 mg/ml.

In some embodiments, Palivizumab is formulated according to the method of the disclosure at a concentration of about 100 mg/ml.

In some embodiments, the antibody is maintained after formulating at a temperature of about −80° C., of about −10° C., of about 25° C., of about 40° C. or of 2-8° C.

The term “antibody is maintained after formulating” refers to the period of storage of the composition comprising the antibody, which has been previously formulated according to the method of the disclosure.

The term “composition is maintained after formulating” refers to the period of storage of the composition comprising the antibody, which has been previously formulated according to the method of the disclosure.

In another embodiment, the antibody is maintained after formulating at a temperature of about −80° C.

In another embodiment, the antibody is maintained after formulating at a temperature of about −10° C.

In another embodiment, the antibody is maintained after formulating at a temperature of about 25° C.

In another embodiment, the antibody is maintained after formulating at a temperature of about 40° C.

In another embodiment, the antibody is maintained after formulating at a temperature of about 2-8° C.

In another embodiment, the composition is maintained after formulating for at least 12 weeks at a temperature of 2-8° C.,

In another embodiment, the composition is maintained after formulating at a temperature of 25° C. for at least 4 weeks,

In another embodiment, the composition is maintained after formulating at temperature of 40° C. for at least 1 week,

In another embodiment, the composition is maintained after formulating at a temperature of −80° C. for at least 24 weeks.

In another embodiment, the composition is maintained after formulating at a temperature of −10° C. for at least 24 weeks.

In another embodiment, the composition is maintained after formulating for at least 6 months at a temperature of 2-8° C.

In another embodiment, the composition is maintained after formulating at a temperature of 25° C. for at least 1 month.

In another embodiment, the composition is maintained after formulating at a temperature of 40° C. for at least 2 weeks.

In one embodiment, the formulated antibody is an anti-PD-1 neutralizing antibody.

In another embodiment, the formulated anti-PD-1 neutralizing antibody is Pembrolizumab or a biosimilar thereof or Nivolumab or a biosimilar thereof.

In some embodiments, Pembrolizumab is formulated according to the method of the disclosure at a concentration of 10 mg/ml, 20 mg/ml, 30 mg/ml, 40 mg/ml, 50 mg/ml, 40 mg/ml, 50 mg/ml, 60 mg/ml, 70 mg/ml, 80 mg/ml, 90 mg/ml, 100 mg/ml, 110 mg/ml, 120 mg/ml, 130 mg/ml, 140 mg/ml, or 150 mg/ml.

In one embodiment, Pembrolizumab is formulated at a concentration of about 25 mg/ml.

In some embodiments, Nivolumab is formulated according to the method of the disclosure at a concentration of about 1 mg/mL, 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml, 21 mg/ml, 22 mg/ml, 23 mg/ml, 24 mg/ml, or 25 mg/ml.

In some embodiments, Nivolumab is present in the formulation of the disclosure at a concentration of 10 mg/ml, 20 mg/ml, 30 mg/ml, 40 mg/ml, 50 mg/ml, 40 mg/ml, 50 mg/ml, 60 mg/ml, 70 mg/ml, 80 mg/ml, 90 mg/ml, or 100 mg/ml.

In one embodiment, Nivolumab is formulated at a concentration of about 10 mg/ml.

In another embodiment, the method does not comprise formulating the antibody with sucrose added to the composition. In another embodiment, the method does not comprise formulating the antibody with polysorbate 80 added to the composition. In another embodiment, the method does not comprise formulating the antibody with sucrose and polysorbate 80 added to the composition. In another embodiment, the method does not comprise formulating the antibody with mannitol added to the composition. In a further embodiment, the method does not comprise formulating the antibody with pentetic acid added to the composition. In another embodiment the method does not comprise formulating the antibody with sodium chloride or/and sodium citrate added to the composition. In another embodiment, the method does not comprise formulating the antibody with mannitol, pentetic acid, sodium chloride or/and sodium citrate added to the composition.

In a further embodiment, the method does not comprise formulating the antibody with a different further antibody added to the composition. In a further embodiment, the method does not comprise more than one antibody added to the composition.

The disclosure is detailed below by means of the following examples which are merely illustrative and by no means limiting the scope of the disclosure.

### EXAMPLES

### Example 1

### Preparation of Antibody Formulations

### Preparation of Palivizumab Antibody Formulations

Palivizumab antibody harvested at 12 days was fully purified. Once purified, the buffer was exchanged by tangential flow filtration and formulated in the different formulations below. As a control, Synagis® (marketed by AstraZeneca) was included into the study as reference product. The palivizumab antibody concentration was set at 100 mg/ml in all the formulations tested. The details of the tested formulations are shown below:

| Formulation | Excipients |
|---|---|
| F0 | 25 mM Histidine, 1.6 mM Glycine, pH 6.2 ± 0.1 |
| F43 | 25 mM Histidine, 16 mM Arginine, pH 6.2 ± 0.1 |
| F44 | 20 mM Histidine/Acetate, 190 mM Sorbitol, 0.10% |
| | PS80, pH 6.2 ± 0.1 |
| F45 | 25 mM Histidine, 1.6 mM Glycine, 100 mM Trehalose, |
| | pH 6.0 ± 0.1 |
| F48 | 25 mm Histidine, 16 mM Arginine, 2 mM Methionine, |
| | pH 6.2 ± 0.1 |

For the preparation of the F0, F43, F45 and F48 formulation buffers, 25 mM L-histidine was added (0.712 g per 500 ml preparation), L-histidine monohydrochloride monohydrate was added to adjust pH to pH 5.8 (1.66 g per 500 ml preparation). 1.6 mM glycine or 16 mM arginine were added afterwards. 2 mM methionine was added for F48. Lastly, 100 mM trehalose dihydrate (18.91 g per 500 ml) was added for F45 formulation and water was adjusted up to 500 ml. For the preparation of F44 formulation buffer, L-histidine was added, and pH adjusted to pH 6.3 with acetate. 190 mM sorbitol was then added and lastly, 0.10% PS80. Water was adjusted up to 500 ml.

### Preparation of Pembrolizumab Antibody Formulations

Pembrolizumab antibody harvested at 12 days was fully purified. Once purified, the buffer was exchanged and formulated in the different formulations below. As a control, Keytruda® (marketed by Merck) was included into the study as reference product. The pembrolizumab antibody concentration was set at 25 mg/ml in all the formulations tested. The details of the tested formulations are shown below:

| Formulation | Excipients |
|---|---|
| F0 | 10 mM Histidine, 70 mg/ml sucrose, 0.02% polysorbate |
| | 80, pH 5.4-5.5 |
| F24 | 25 mM Histidine, 1.6 mM Glycine, 100 mM Trehalose, |
| | pH 5.4-5.5 |
| F25 | 18 mM Acetate, 4.6 mM Sorbitol, 0.01% PS20, |
| | pH 5.4-5.5 |
| F26 | 15 mM Histidine, 5 mM Acetate, 190 mM Sucrose, |
| | 0.10% PS80, pH 5.4-5.5 |

For the preparation of F24 formulation buffer, 25 mM L-histidine was added, L-histidine monohydrochloride monohydrate was added to adjust pH to pH 5.4. 1.6 mM glycine was added afterwards. Lastly, 100 mM trehalose dihydrate was added and water was adjusted up to 500 ml.

For the preparation of F25 formulation buffer, 18 mM acetic acid glacial was added and the pH adjusted to pH 5.4-5.5 with NaOH. 4.6 mM sorbitol was then added. Lastly, 0.01% PS20 was added, and water adjusted up to 500 ml.

For the preparation of F26 formulation buffer, L-histidine was added, and pH adjusted to pH 5.4 with 5 mM acetate. 190 mM sucrose was then added and lastly, 0.10% PS80. Water was adjusted up to 500 ml.

### Preparation of Nivolumab Antibody Formulations

Nivolumab antibody harvested at 12 days is fully purified. Once purified, the buffer is exchanged and formulated in the different formulations below. As a control, Opdivo® (marketed by Bristol-Myers Squibb) is included into the study as reference product. The nivolumab antibody concentration is set at 10 mg/ml in all the formulations tested. The details of the tested formulations are shown below:

| Formulation | Excipients |
|---|---|
| F0 | 30 mg Mannitol, 0.008 mg pentetic acid, 0.2 mg |
| | polysorbate 80, 2.92 mg sodium chloride, |
| | 5.88 mg sodium citrate dihydrate, pH 6.0 |
| F24 | 25 mM Histidine, 1.6 mM Glycine, 100 mM |
| | Trehalose, pH 5.4 ± 0.1 |

For the preparation of F24 formulation buffer, 25 mM L-histidine is added, L-histidine monohydrochloride monohydrate is added to adjust pH to pH 5.4. 1.6 mM glycine is added afterwards. Lastly, 100 mM trehalose dihydrate is added and water is adjusted up to 500 ml.

The samples were placed at 40° C., 25° C., 2-8° C., −20° C. and −80° C. to elucidate the colloidal and conformational stability of the molecules. Visual inspection, protein concentration, osmolality, conductivity and pH measurements were performed to monitor the stability of the protein. Size Exclusion chromatography (SEC) and Ion Exchange chromatography (IEX) were used to characterize protein stability. The analytical panel and time points used in the study are:

| Conditions/ | Analytical |
|---|---|
| Time | panel |
| T0 | Visual Inspection, concentration, pH, conductivity, osmolality, |
| | SEC, IEX, CE-SDS NR, CE-SDS R, μDSC, Binding ELISA |
| T2-8° C. (12, 24, 36, 52 w) | Concentration, pH, SEC, cIEF, CE-SDS NR, CE-SDS R |
| T25° C./60% HR (4, 8, 12, 16, 20 w) | Concentration, pH, SEC, IEX, CE-SDS NR, CE-SDS R, μDSC |
| T40° C. (1, 2 w) | Concentration, pH, SEC, IEX, CE-SDS NR, CE-SDS R |
| T −80° C. (24, 52 w)/T −20° C. (24, | Concentration, pH, SEC, IEX, CE-SDS NR, CE-SDS R |
| 52 w) | |

### Example 2

### Protein Concentration, pH, Conductivity and Osmolality Determination

Protein concentration was assessed by means of OD280. UV Lambda 25 Perkin Elmer UV visible spectrophotometer was used for protein concentration measurements (280 nm wavelength). 10 mm quartz cuvettes were used, with a volume of 1000 μL (Hellma® Analytics). The corresponding buffer of each formulation was used as OD 280 blank. The protein concentration at T0 was comparable in all formulations. The protein concentration was constant during the stability period of 40° C. 2 weeks, 25° C. 16 weeks, 2-8° C. 36 weeks, −80° C. 24 weeks and −20° C. 24 weeks. The pH was determined by using a VWR pH 1100L, following the manufacturer instructions. The pH accuracy is of 0.05. Conductivity was determined by using a Mettler Toledo Seven2GO S3 device, following the manufacturer instructions. Osmolality was determined by using the Osmo1® Micro-Osmometer, following the manufacturer instructions.

### Palivizumab

The protein concentration at T0 was comparable in all palivizumab formulations. Palivizumab F0 and F45 formulations comprised a concentration of the protein of 102.7 mg/ml and 99.1 mg/ml, respectively. The protein concentration was constant during the stability period of 40° C. 2 weeks, 25° C. 16 weeks, 2-8° C. 36 weeks, −80° C. 24 weeks and −20° C. 24 weeks. The pH at T0 for F45 was 5.90 and for F0, F43, F44 and F48 pH 6.24, 6.23, 6.28 and 6.19, respectively. The conductivity values determined were 1706 μS/cm for F0, 1720 μS/cm for F43, 1100 μS/cm for F44, 1955 μS/cm for F45 and 1872 μS/cm for F48. The osmolality values determined for F0 were 53 mOsm/kg, for F43 111 mOsm/kg, for F44 256 mOsm/kg, for F45 210 mOsm/kg and for F48 100 mOsm/kg. Therefore, for palivizumab formulations, concentration, pH, conductivity and osmolality were constant during the stability time points for all the formulations.

### Pembrolizumab

The pH at T0 for F0, F24, F25 and 26 were pH 5.33, 5.41, 5.47 and 5.41, respectively. The conductivity values determined were 940 μS/cm, 1794 μS/cm, 1207 μS/cm and 1095 μS/cm, respectively. The osmolality values determined were 263 mOsm/kg, 193 mOsm/kg, 324 mOsm/kg and 265 mOsm/kg, respectively. Visual inspection, concentration and pH at T0 were comparable for all the formulations tested. For pembrolizumab formulations, conductivity and osmolality were also constant during the stability time points for all the formulations.

### Example 3

### Micro Differential Scanning Calorimetry (μDSC)

The conformational stability is a key parameter for prediction of long-term stability of proteins and was used herein for the formulation candidates. A Microcal PEAQ-DSC automated instrument was used for the determination. The antibody in solution is in equilibrium between its native (folded) and denatured (unfolded) conformations. The higher the thermal transition midpoint (Tm), the more stable the molecule. DSC measures the enthalpy (ΔH) of a sample that results from heat-induced denaturation. It is also used to determine the change in heat capacity (ΔCp) of denaturation. The ΔH is calculated as the area under the endothermic curve (kcal/mol or KJ/mol). The Tonset temperature is defined as the temperature at which the protein starts to denature due to changes in folding. Tm1, Tm2 and Tm3 indicate the melting points of the antibody solutions. Tm1, Tm2 and Tm3 of palivizumab F0, F43, F44, F45 and F48 formulations were quantified.

Table 1 below summarizes the average of the different Tm values:

| | μDSC | | | | | |
|---|---|---|---|---|---|---|
| | | | | | | Thalf |
| | Tonset | (ΔH)(kcal/ | Tm1 | Tm2 | Tm3 | Main Peak |
| Sample | (° C.) | mol) | (° C.) | (° C.) | (° C.) | (° C.) |
| Synagis | 64.6 | 1196.7 | 70.8 | 85.0 | 88.2 | 8.09 |
| F0 | 64.9 | 1196.7 | 71.0 | 85.0 | 88.2 | 8.10 |
| F43 | 62.9 | 1270.0 | 69.1 | 84.5 | 88.8 | 10.46 |
| F44 | 65.5 | 1276.7 | 71.2 | 85.5 | 88.7 | 8.02 |
| F45 | 63.2 | 1126.7 | 68.9 | 84.7 | 89.2 | 7.28 |
| F48 | 62.9 | 1330.0 | 68.9 | 84.5 | 88.8 | 10.31 |

Tm obtained for each formulation showed comparable values between formulations demonstrating that F43, F44, F45 and F48 formulations provide similar stability to the molecule compared with F0 and the reference product formulation.

### Example 4

### Size Exclusion Chromatography (SEC)

SEC was used to characterize protein aggregation, because it shows clear separations between aggregates, antibody monomers and fragments. For SEC, a high-performance liquid chromatography system and a Column Super SW3000; 4,6×300 mm (4 μm) (TOSOH Bioscience) were used. Each assay included a reference standard preparation, which was injected 3 times at the beginning and 3 times at the end of the analysis sequence. In addition, the reference standard preparation was injected 1 time for every 10 sample injections. The reference standard preparation was palivizumab antibody in F0 formulation or Synagis® formulation, pembrolizumab antibody in F0 formulation or Keytruda®, or nivolumab antibody in F0 formulation or Opdivo®. A blank preparation (mobile phase) was performed and included in each analysis sequence. Each independent sample preparation was injected in duplicates. The samples were prepared at a concentration of 1 mg/mL, using mobile phase as diluent. The samples were filtered through a 0.2 μm membrane and 30 μl of each sample was injected.

Considering all injections of the reference standard, the mean, standard deviation (SD) and RSD (%) of the following parameters were calculated:
Total integrated area,
Percentage area of Main Peak (monomer),
Retention time of Main Peak (monomer),
Number of theoretical plates of Main Peak (monomer),
Resolution of Main Peak (monomer),
Symmetry factor of the main peak of Main Peak (monomer),
Sum of percentage area of the peaks with less retention time (pre-peaks, aggregates or HMW),
Sum of percentage area of the peaks with longer retention time (post-peaks, fragments or LMW).

The mean and the % RSD of the following parameters was also calculated for each of the samples:
Total integrated area of the peaks.
Area percent of the main peak, pre-peaks and post-peaks.
Retention time of the main peak.

The purity of the sample corresponds to the percentage area of the main peak.

Calculations with relative peak areas (e.g., for peak grouping) were performed using initial values for relative peak areas with three decimal places. The initial values used for calculations and the calculated results are reported rounded to one decimal place. The initial values used for calculations and the calculated results are reported rounded to one decimal place. The formulation samples were incubated at 40° C. for 2 weeks, at 25° C. for 4 weeks, 8 weeks, 12 weeks and 16 weeks, at 2-8° C./−20° C. and −80° C. for 12 weeks, 24 weeks and 36 weeks. The high molecular weight species (dimers, trimers or tetramers) were calculated by summing the areas of the different peaks eluting at an earlier time than the monomer and referring them to the sum of the areas of total peaks.

### Palivizumab

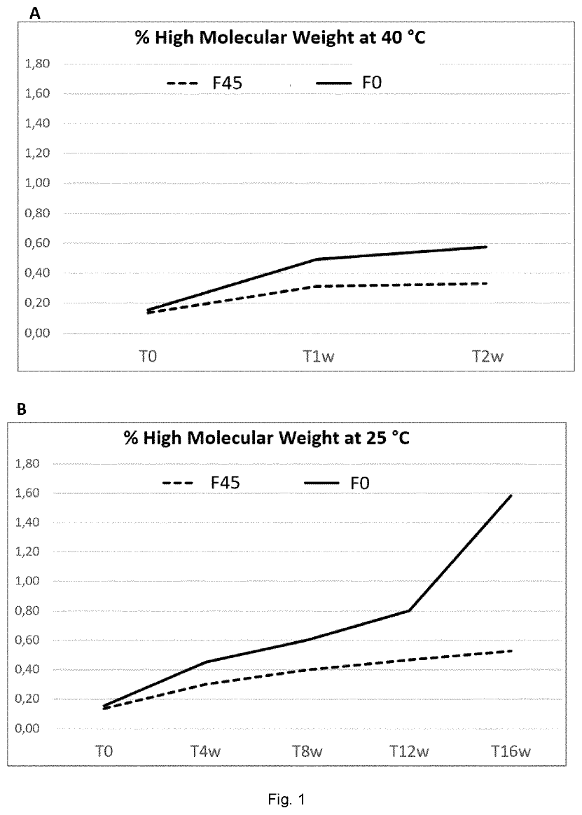

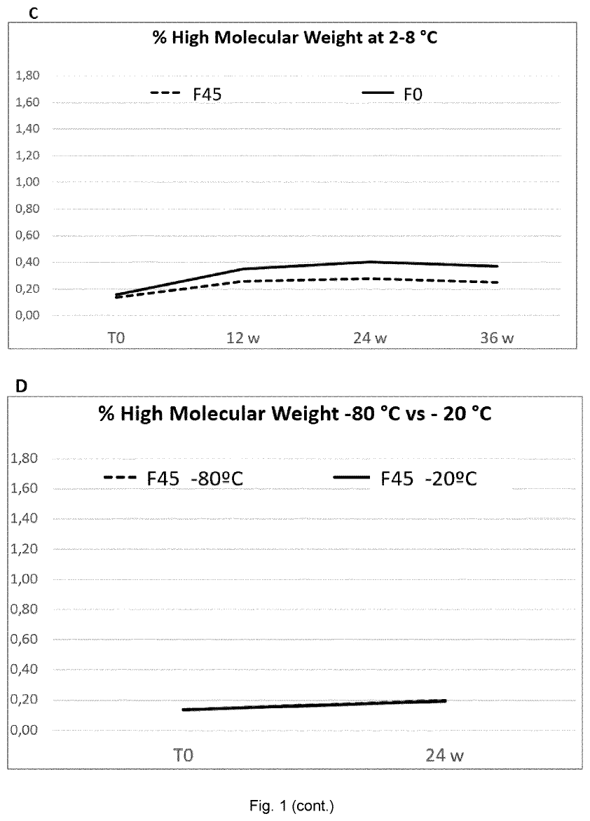

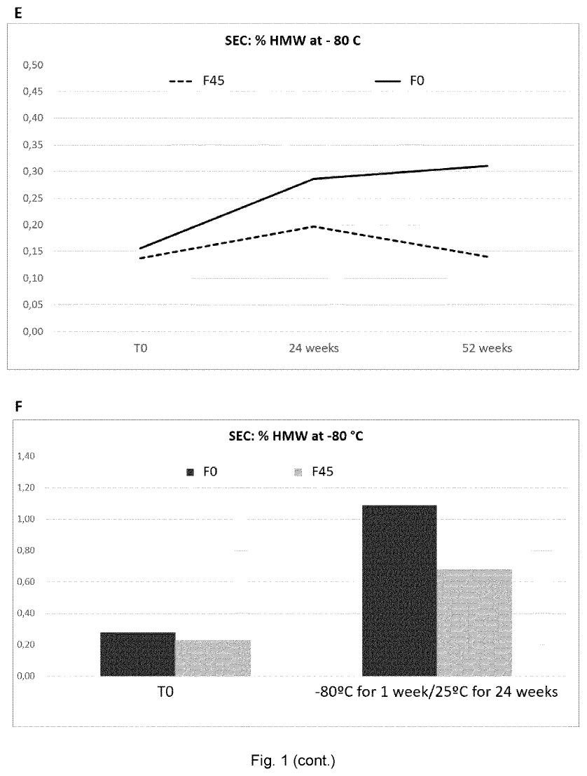

F45 did not show significant aggregation upon storage at 40° C. for up to 2 weeks, as no significant changes in high molecular weight species were detected. See FIG. 1A. Similarly, F45 did not show significant aggregates upon storage at 25° C. for up to 16 weeks. See FIG. 1B. Compared to F45, F0 showed higher evidence of aggregate formation, as the percentage of high molecular weight species significantly increased for F0 formulation from week 12, as shown in FIG. 1B. F45 did not show higher-order of aggregates when stored at 2-8° C. and −80° C./−20° C., as no significant changes in high molecular weight species was detected for 24 weeks and even for up to 52 weeks at −80° C. F0 showed an increased level in aggregates when maintained at −80° C. for up to 52 weeks, compared to F45. See FIGS. 1C, 1D and 1E. F45 and F0 were both stored at-80° C. for one week followed by a six-month incubation at 25° C. for 24 weeks. The molecular aggregates were quantified by SEC. As shown in FIG. 1F, the amount of high molecular weight species in F45 was significantly lower than in F0 in these conditions.

### Pembrolizumab

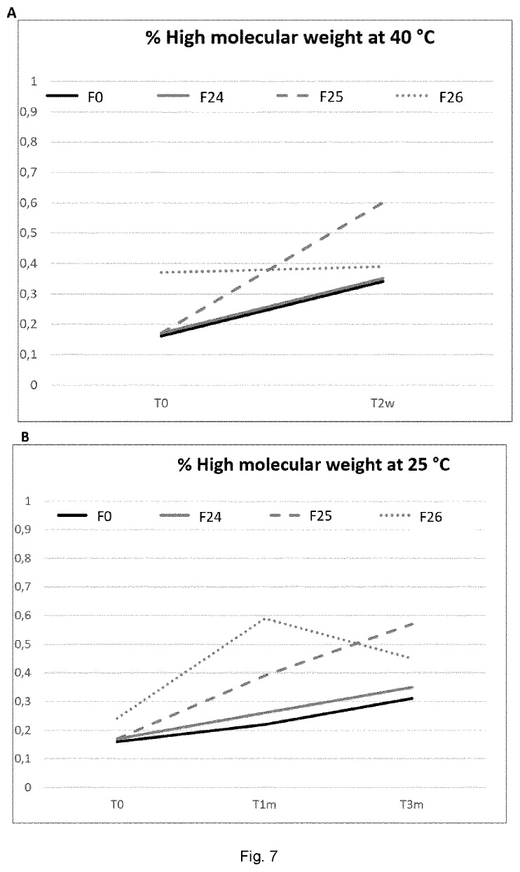

F24 and F26 did not show aggregation upon storage at 40° C. for up to 2 weeks, as no significant changes in high molecular weight species were detected. The changes were comparable to F0 formulation. See FIG. 7A. F25 showed a higher degree of aggregation, as the high molecular weight species increased compared to T0.

Similarly, F24 did not show significant aggregates upon storage at 25° C. for up to 3 months. See FIG. 7B. The aggregates levels were very similar to the aggregates observed for F0 formulation. Compared to F24, F25 and F26 showed evidence of aggregate formation, as the percentage of high molecular weight species significantly increased at 3-months time, as shown in FIG. 7B. The table below shows the difference in the % of high molecular weight (HMW) species between the initial time point (T0) and the end time point (2 weeks, 1 month or 3 months). As shown in the table, F25 and F26 showed higher level of aggregation compared to Keytruda®, F0 and F24 formulations.

| | Δ HMW (T0 − | Δ HMW (T0 − | Δ HMW (T0 − |
|---|---|---|---|
| | T 40 C. 2 w) | T 25 C. 1 m) | T 25 C. 3 m) |
| F0 | 0.18 | 0.06 | 0.15 |
| F24 | 0.18 | 0.09 | 0.18 |
| F25 | 0.43 | 0.22 | 0.4 |
| F26 | 0.02 | 0.22 | 0.21 |
| Keytruda ® | 0.12 | −0.03 | 0.02 |

### Example 5

### Ion Exchange Chromatography

Ion exchange chromatography was used to characterize protein stability. A weak cation exchange column was used (Dionex Propac WCX-10 column, 4.0 mm i.d.×250 mm). Each assay included a reference standard preparation, which was injected 3 times at the beginning and 3 times at the end of the analysis sequence. In addition, is injected 1 time for every 10 sample injections. The reference standard preparation was palivizumab antibody in F0 formulation or Synagis® formulation. The reference standard preparation was palivizumab antibody in F0 formulation or Keytruda® formulation. The samples were prepared at a concentration of 1 mg/mL, using mobile phase as diluent. The samples were filtered through a 0.2 μm membrane and 40 μl of each sample was injected. The equipment software (Empower 3) was used to integrate the total area of the main peak, the peak areas with the shortest retention time (pre-peak or acidic forms) and the peak areas with the longest retention time (post-peak or basic forms). Considering all the injections of the reference standard, the mean, standard deviation (SD) and the % Relative Standard Deviation (RSD) of the following parameters were calculated:
Total integrated area, percentage area.
Retention time.
Number of theoretical plates.
Width 50%.
Start P/V.
Percentage area of the peaks with less retention time (pre-peaks or acid forms).
Percentage area of the peaks with the longest retention time (post-peak or basic forms).

The mean and the % RSD of the following parameters was also calculated for each of the samples:
Total integrated area of the peaks.
Percentage area of the main peak, pre-peak and post-peak.
Retention time of the main peak.

The purity of the sample corresponds to the percentage area of the main peak.

Calculations with relative peak areas (e.g., for peak grouping) were performed using initial values for relative peak areas with three decimal places. The initial values used for calculations and the calculated results are reported rounded to one decimal place. The initial values used for calculations and the calculated results are reported rounded to one decimal place.

The formulation samples were incubated at 40° C. for 2 weeks, at 25° C. for 2 weeks and 12 weeks, at 2-8° C./−20° C. and −80° C. for 2 weeks, 12 weeks and 24 weeks. The percentage of acidic and basic species was calculated by summing the areas of the acidic peaks (pre-peaks) or the basic peaks (post-peaks) and referring them to the sum of the areas of total peaks.

### Palivizumab

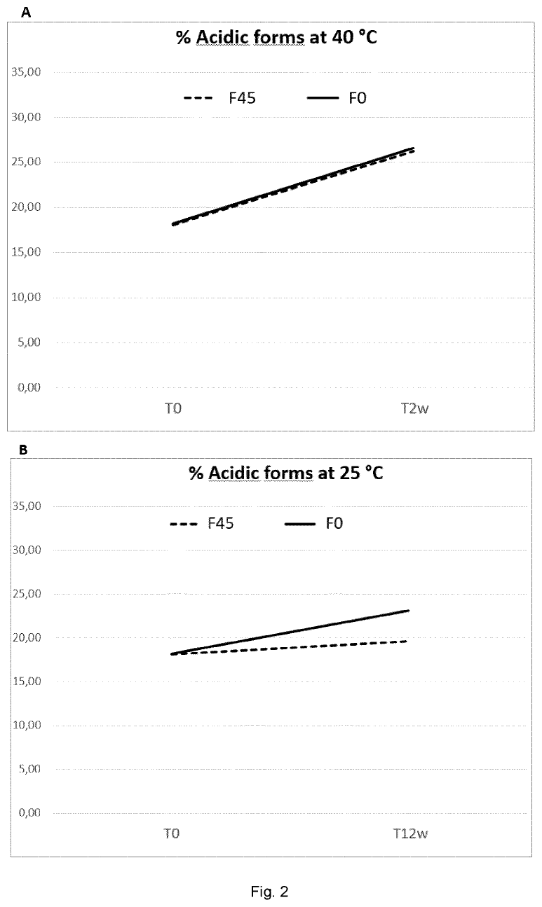

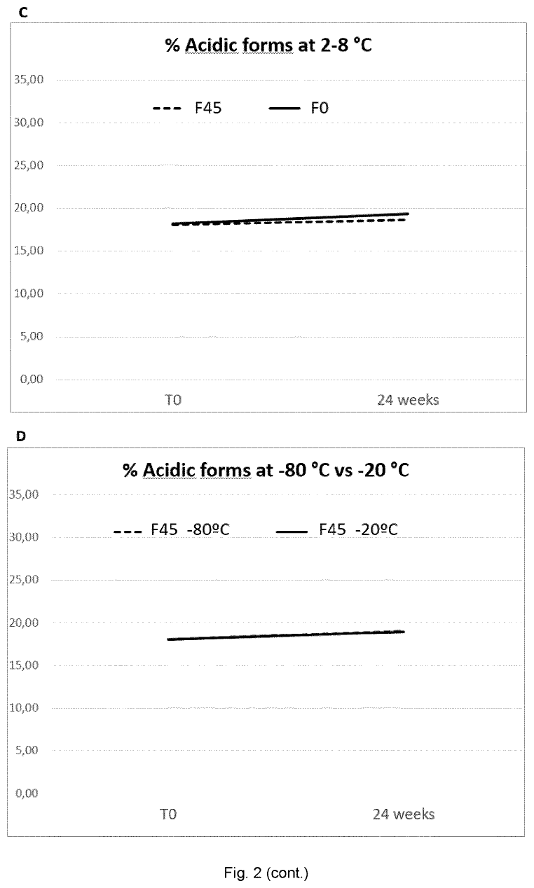

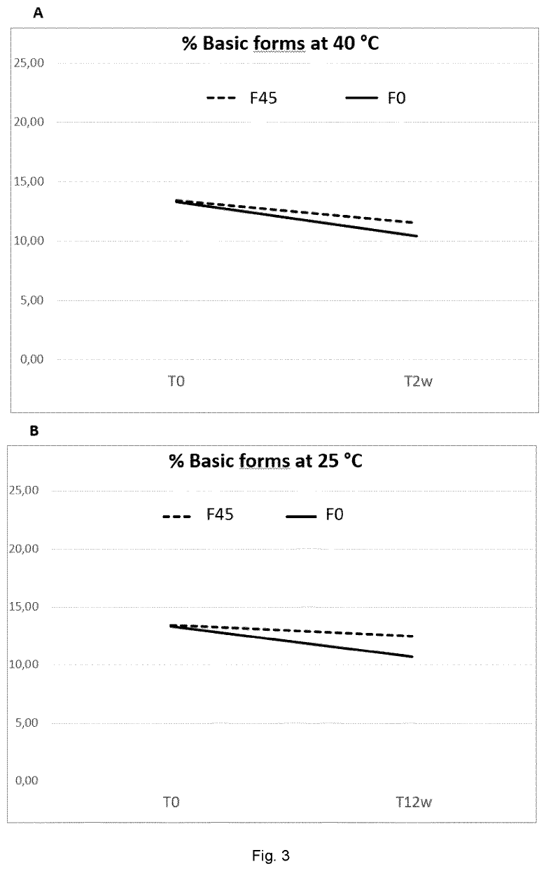

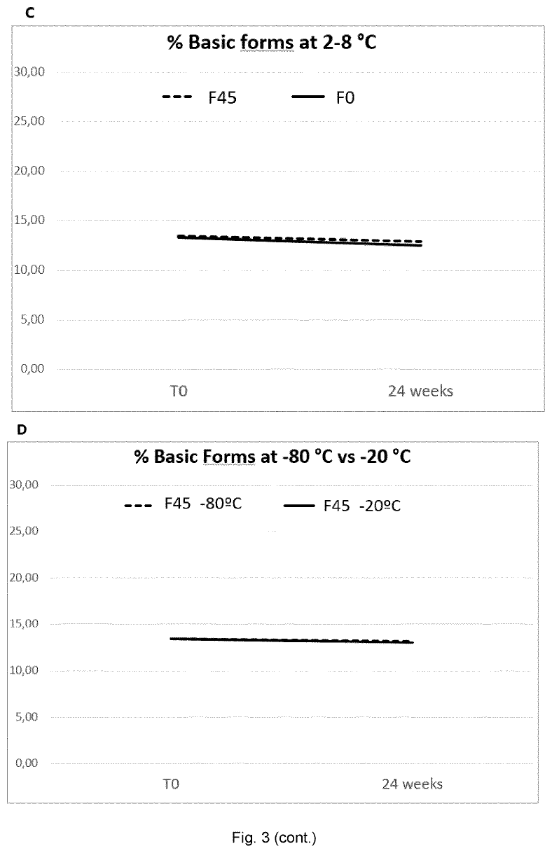

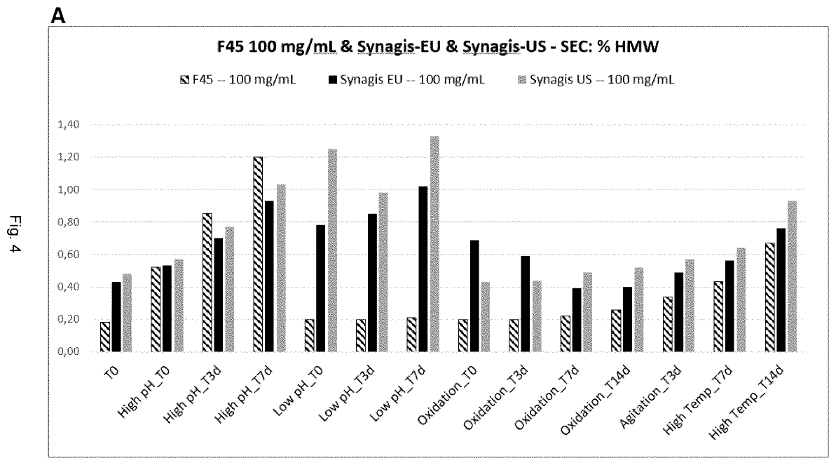

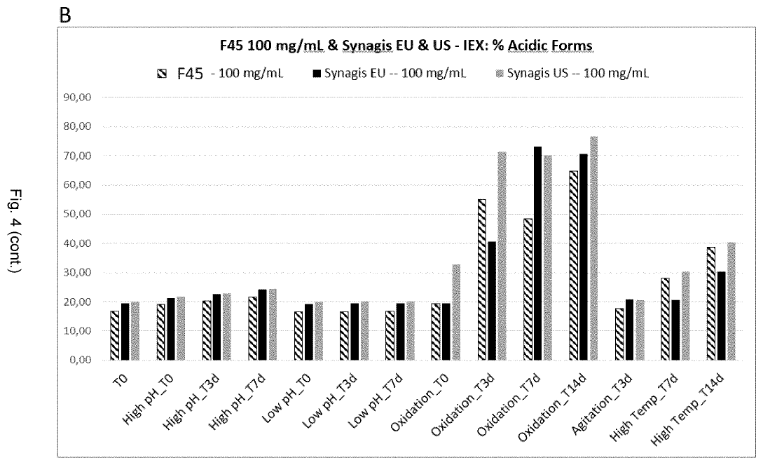

F45 showed excellent stability upon storage at 25° C. for 12 weeks, as no significant changes in acidic species was quantified. The percentage of acidic forms increased around 5% for F0 formulation, as shown in FIG. 2B. F45 also showed excellent stability when stored at 2-8° C. and −80° C./−20° C., as no significant changes in acidic forms was detected for 24 weeks. See FIGS. 2C and 2D. A shift to acidic species was detected for both F45 and F0 formulations when stored for 2 weeks at 40° C., as shown in FIG. 2A. The change was comparable for both F0 and F45 formulations. F45 showed very good stability upon storage at 25° C. for 12 weeks, as no significant change in basic species was observed, whereas a decrease in basic species was detected with F0 formulation. See FIG. 3B. Upon storage at 40° C. a slight decrease in the % of basic forms was observed with F45. The shift was also observed for F0 formulation, to a higher extent. See FIG. 3A. F45 also showed excellent stability when stored at 2-8° C. and −80° C./−20° C., as no significant changes in basic forms was detected for 24 weeks. See FIGS. 3C and 3D.

### Pembrolizumab

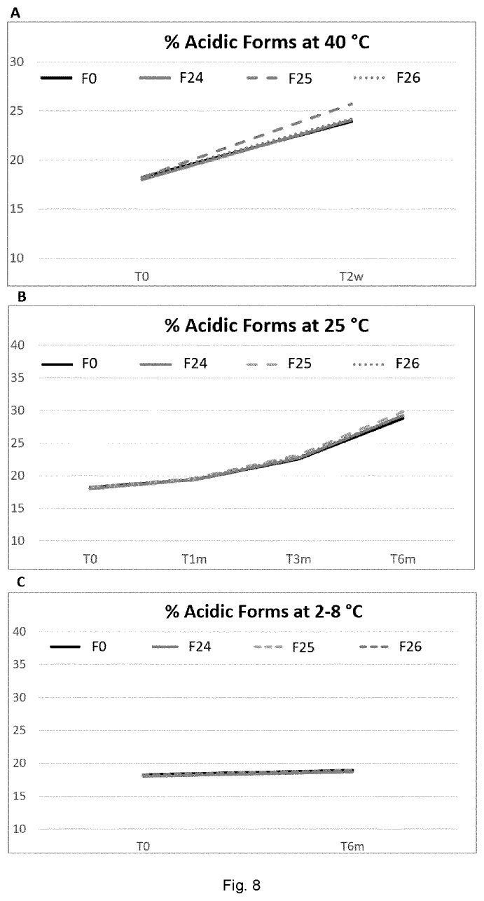

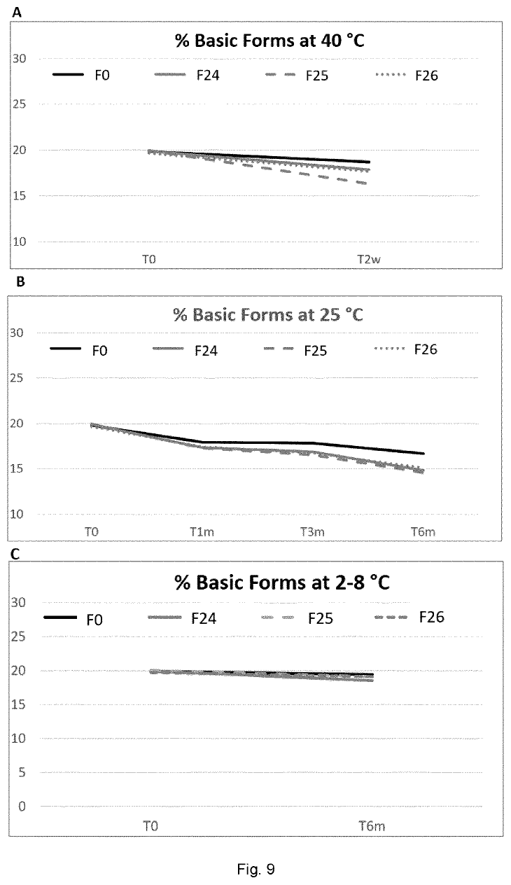

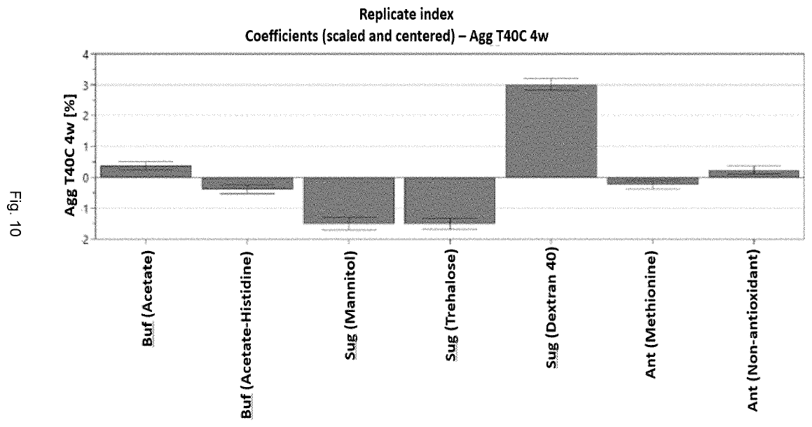

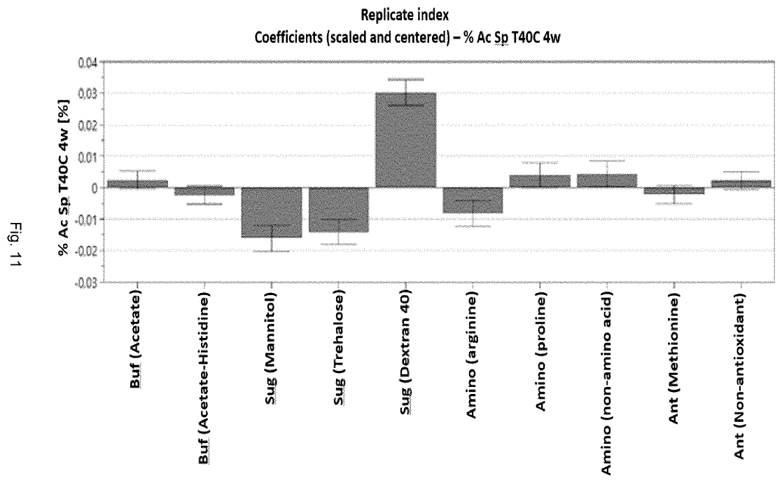

A shift to acidic species was detected for both F24 and F26 formulations when stored for 2 weeks at 40° C., as shown in FIG. 8A. The change was comparable to F0. The shift to acidic species was higher for F25. A shift to acidic forms was also detected for all the tested formulations at 25° C. for up to 6 months, though the shift was smaller up to 3 months, as shown in FIG. 8B. F24, F25 and F26 showed excellent stability upon storage at 2-8° C. for up to 6 months, as no significant changes in acidic species was quantified. The levels were comparable to F0 formulation, as shown in FIG. 8C. F24 and F26 showed very good stability upon storage at 40° C. for 2 weeks, as no significant change in basic species was observed (comparable to F0 formulation), whereas a decrease in basic species was detected with F25 formulation. See FIG. 9A. Upon storage at 25° C. a slight decrease in the % of basic forms was observed with all formulations. The shift was also observed for F0 formulation. See FIG. 9B.

F24, F25 and F26 also showed excellent stability, and comparable to F0, when stored at 2-8° C., as no significant changes in basic forms was detected for 24 weeks. See FIG. 9C.

### Example 6

Detection of Acidic and Basic Species by cIEF.

The aim of capillary isoelectric focusing (cIEF) is to determine the isoelectric point (pI) and the heterogeneity of charge isoforms of a protein that are caused by posttranslational modifications. The power of cIEF lies in the high resolving electropherograms allowing a precise and reproducible relative quantification of the charge isoforms. The PA 800 Plus Pharmaceutical Analysis System (Sciex, Beckman Coulter) was used for the determination of the pI by cIEF and the method High Resolution Integration to analyze the data. All data was integrated using 32 Karat™ software.

For each sample, the following calculations were performed:
Area for main peak, basic and acidic forms with one decimal,
Main peak pI with one decimal,
Correlation coefficient (R2) of linear curve of pI markers with two decimals, and
Theoretical plate number of pI 10.0 without decimals.

A slight increment in acidic species during stress and accelerated conditions was observed for all molecules. For palivizumab, the % of acidic forms slightly increased for F44, F45 and F48 formulations when maintained at 25° C. for up to 12 weeks and was very similar to the % of acidic forms of F0. For F43, the increase was more significant. A similar behaviour was observed at 40° C., being F43 the one with the highest increase. Data no shown. For palivizumab, no significant changes in the % of basic species were observed for any of the stability conditions (25° C., 40° C. and 2-8° C.). Data no shown.

### Example 7

### Capillary Electrophoresis (CE) in Non-Reducing (CE-NR) and Reducing Conditions (CE-R)

Capillary gel electrophoresis with sodium dodecyl sulfate (CE-SDS, CGE-SDS) is an analytical technique able to separate proteins based on their mass within a high viscosity electrolytic solution (sieving matrix) under the influence of an electric field. Samples are denatured by heat and treated with sodium dodecyl sulfate (SDS) in order to provide a high negative charge density to each species so they can be separated based on their migration velocity differences resulted by their relative hydrodynamic volumes. Separation is performed in a bare fused silica capillary tube with a 50 μm of internal diameter. For the non-reduced analysis (CE-NR), the native protein is treated prior to separation with SDS and Iodoacetamide, an alquilating agent, is added to avoid disulfide scrambling and free cysteine related disulfide breakage. For reduced analysis, the sample is treated with SDS and tris(2-Carboxyethyl)Phosphine to reduce the native protein structure and to allow the identification of the distinct protein subunits. A capillary electrophoresis equipment from Beckman Coulter PA 800 was used for the analysis.

For CE-NR, the following parameters were analyzed:
% RSD corrected area percent for the sum of impurities
% RSD corrected area percent of main peak (IgG)

For CE-R, the following parameters were analyzed:
% RSD corrected area percent for the sum of impurities
% RSD corrected area percent of NGHC peak
% RSD corrected area percent of HC

Therefore, CE-NR was used to assess the antibody fragmentation.

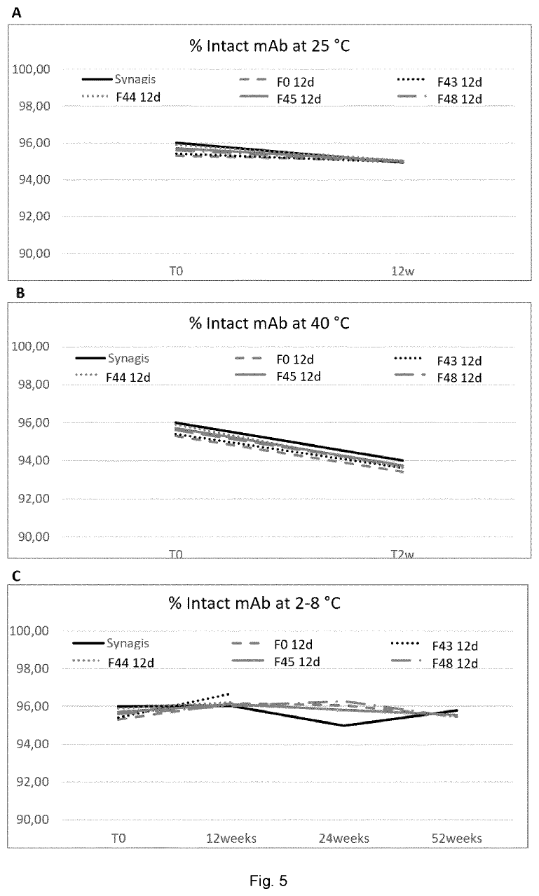

For palivizumab, a slight decrease in the % of intact antibody was observed in the CE-NR results for formulations Synagis®, F0, F43, F44, F45 and F48 at 25° C. (FIG. 5A) and 40° C. (FIG. 5B) for up to 12 weeks and 2 weeks, respectively. However, no significant changes were detected in the % of intact antibody during the stability period of 1 year at 2-8° C. for any of the formulations tested (FIG. 5C). Similarly, for palivizumab a slight increase in the % of fragments of antibody was observed in the CE-NR results for formulations Synagis®, F0, F43, F44, F45 and F48 at 25° C. and 40° C. for up to 12 weeks and 2 weeks, respectively. However, no significant changes were detected in the % of fragments of antibody during the stability period of 1 year at 2-8° C. for any of the formulations tested (Data not shown).

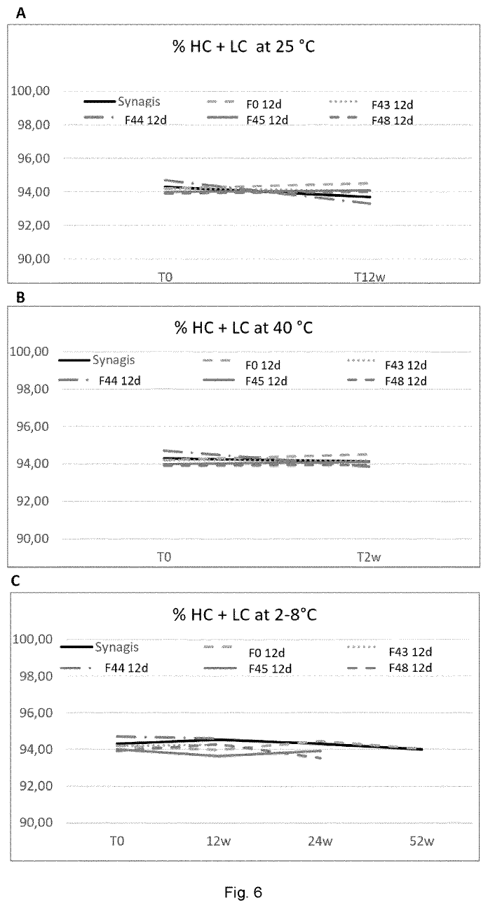

Reducing CE (CE-R) analysis allowed quantification of the heavy (HC) and light chains (LC) and degradants in the antibody samples. Partial reduction with 50 mM Tris(2-carboxiethyl)-phosphine (TCEP) was performed, at 95° C. for 5 minutes. For palivizumab, no significant changes in the % of heavy chains and light chains in the antibody was observed in the CE-R results for formulations Synagis®, F0, F43, F44, F45 and F48 at 25° C. (FIG. 6A) and 40° C. (FIG. 6B) for up to 12 weeks and 2 weeks, respectively. No significant changes were observed for all the formulations at 2-8° C. for up to 52 weeks (FIG. 6C). Out of all the formulations tested, F45 and F48 formulations are the more stable formulations, showing very little changes in the % of HC and LC throughout the time at the temperatures tested.

CE-R also allows the quantification of non-glycosylated heavy chain (NGHC). No significant changes in the % of NGHC were detected for palivizumab F0, F43, F44, F45 and F48 formulations during the stability period of 1 year at 2-8° C. During stress and accelerated conditions (incubation at 40° C. up to 2 weeks and at 25° C. up to 12 weeks), the same results were observed. Data not shown.

### Example 8

### Forced Degradation Experiment

The study design of the forced degradation experiment is described in Table 2 and was used to determine the stability of the formulations in different stress conditions. Palivizumab F45 formulation and Synagis® samples from EU batches or US batches were used. The samples at 2-8° C. were transferred from the refrigerator and allowed to reach room temperature. The material was then placed under the different forced degradation conditions described below. After the samples were stressed, each time point was analysed. The following parameters were analysed for each sample, each condition and different time points: visual inspection, protein concentration, pH, conductivity, osmolality, aggregation levels by SEC, charge variants by IEX and cIEF and impurities analysed by CE-SDS in non-reduced conditions, conformational stability analysed by μDSC and biological activity analysed by Protein F binding ELISA.

TABLE 2
Summary of forced degradation stability conditions.

| | | Stress | Major Degradation |
|---|---|---|---|
| Test | Condition | intensity | pathways |
| High | 45° C. dry incubation | 7, 14 days | Aggregates, fragments, |
| Temperature | | | oxidation, deamidation |
| Oxidation | Addition 0.05% H₂O₂ and | (0), 3, 7, 14 | Oxidation of methionine |
| | incubation 5 ± 3° C. | days | and tryptophan residues |
| Agitation | Stirring 600 RPM RT | 3 days | Aggregates |
| (stir) | | | |
| Low pH | pH transfer to pH 3 by dialysis (max | (0), 3, 7 | Aggregates, aspartic acid |
| (Acidic) | 12 hours) and incubation 5 ± 3° C. | days | isomerization |
| High pH | pH transfer to pH 9 by dialysis (max | (0), 3, 7 | Aggregates, deamidation |
| (Basic) | 12 hours) and incubation 5 ± 3° C. | days | |

Briefly, description of each stress condition is indicated as follow:
Temperature stress—45° C. stress conditions. The material was introduced in an incubator set at 45° C. for 7 and 14 days.
pH stress—low and high pH.

Low pH stress: Molecule was exposed to low pH buffering exchange in a buffer with pH of 3. After dialysis, the molecule was stored in a chamber at 2-8° C. and analysed at days 0, 3 and 7.

High pH stress: Molecule was exposed to high pH buffering exchange in a buffer with pH of 9. After dialysis, the molecule was stored in a chamber at 2-8° C. and analysed at days 0, 3 and 7.
Oxidation stress—addition of an oxidizing agent. The material robustness against oxidation was evaluated through the addition of an oxidative agent directly into the solution. 0.05% of H₂O₂ was spiked on the sample solution and it was analysed at day 0, 3, 7 and 14 days. The material was stored in a chamber at 2-8° C. and analysed at days 0, 3, 7 and 14.
Mechanical stress—agitation at 600 rpm for 3 days at RT (20-22° C.). The samples were exposed to an agitation condition in a magnetic agitator at RT to evaluate the impact of the stability.

### Results

Levels of antibody concentration were found to be similar for all forced degradation conditions compared with T0.

The pH values under the forced degradation conditions did not change except for the high pH and low pH, which was done intentionally.

No changes in the values of conductivity and osmolality were observed for any of the forced degradation conditions except for the high pH and low pH conditions.

The levels of aggregation and main peak were measured by SEC. The levels of aggregates (% of high molecular weight species) remained unchanged for the oxidation condition for all the products. However, an increment in the levels of aggregates were observed for the high pH and High Temperature conditions for all the molecules. In the case of high pH, the levels of aggregates reached values between 1%-1.5% after 7 days; and for High Temperature, the levels reached were of 0.6-1.0%. These values were comparable for both Synagis® and F45 samples. Interestingly, for the low pH condition, aggregation was only observed for the Synagis® samples (for both the EU and US samples), reaching values of 1-1.3%, whereas the F45 sample aggregates remained stable at days 0, 3 and 7, at values around 0.2%. See FIG. 4A.

Charge variants were analyzed by two orthogonal techniques IEX and cIEF. The results obtained were similar for both techniques and the same trend was observed for all the molecules.

Charge variants were analyzed by IEX. Initial level of charges variants was similar between F45 and Synagis®. The level of charge variants remains unchanged for High pH, Low pH and Agitation conditions. Oxidation led to some reduction in main peak, which are compensated by increase in acidic and basic species. The profile under oxidation conditions showed a significant change in all the molecules. Increase in acidic species was observed for all the products under the high temperature condition. After 14 days the levels of acidic species reached values of around 40% for F45 and 30-40% for Synagis® (See FIG. 4B).

The temperature-based degradation was similar between F45 and Synagis®. Overall, F45 was found comparable to Synagis® upon the stress conditions tested.

Charge variants were analyzed by cIEF and IEX. As observed for IEX, oxidation and High Temperature were the conditions in which the charge variants showed modifications compared to T0. In the case of oxidation, an extreme increase in acidic species and decrease in mean peak and basic species were observed. Increase in acidic species was observed for all the products under the high temperature condition. After 14 days the levels of acidic species reached values of around 40% for F45 and similar for Synagis®. The temperature-based degradation was similar between F45 and Synagis®. Therefore, F45 was very similar to Synagis® upon the stress conditions tested. Data not shown.

Conformational stability was analyzed by μDSC. No significant changes in the Tm values were observed for the agitation and high temperature conditions for all the samples. Substantial modifications in the values of the Tm were observed under the low pH and oxidation condition indicating less conformational stability of the molecules under these conditions. In addition, slight changes under the High pH condition were observed for all the products. Data not shown.

Overall, under the stress conditions tested, the candidate F45 showed a highly comparable behavior compared to Synagis®.

### Example 9

### Biological Activity Measured by Protein F or Competitive Binding ELISA.

### Palivizumab

A plastic plate is covered with a fixed amount of Protein F and left to be coated 16±3 h at 5±3° C. After incubating the plates for a few minutes with a commercial reagent (Superblock), different concentrations of the palivizumab reference standard (Synagis®) and F0, F45 samples (primary antibody) are added onto the plate (2). Afterwards, a polyclonal anti-human IgG secondary antibody conjugated with HRP (horseradish peroxidase) which recognizes the Fc region of palivizumab is added, attaching to the antigen-primary antibody complex (3). Finally, the formation of complexes is observed by the addition of HRP substrate, 3,3 ‘, 5,5’-Tetramethyl benzidine (TMB), and its subsequent stop with Sulfuric acid 1 M, generating a colorimetric product detectable at 450 nm (4). The intensity of the signal is proportional to the amount of palivizumab antibody associated with Protein F.

No noticeable differences were observed for High pH, Low pH, Agitation and High temperature conditions for any of the molecules and the results were comparable to F0, in which all the values were in a range from 80 to 110% of binding.

### Pembrolizumab and Nivolumab

A plastic plate is covered with a fixed amount of PD-1 and left to be coated 16±4 h at 5±3° C. After incubating the plates for 1 hour with a commercial reagent (Blotto 5X), different concentrations of the reference standard and pembrolizumab or nivolumab samples (primary antibody) mixed with a fixed amount of PD-L1 are added onto the plate and incubated for 1 h. Afterwards, an anti-PD-L1 antibody conjugated with HRP (horseradish peroxidase) is added, joining to PD-L1. Finally, the formation of complexes is revealed by the addition of HRP substrate, 3,3 ‘, 5,5’-Tetramethyl benzidine (TMB) and its subsequent stop with Sulfuric acid 1 M, generating a colorimetric product detectable at 450 nm (4). The intensity of the signal is inversely proportional to the amount of pembrolizumab or nivolumab samples associated with PD-1. Comparing the dose-response curves generated with the reference standard and the biosimilar samples, the relative binding potency of the biosimilar to PD-1 can be calculated.

No noticeable differences were observed for High pH, Low pH, Agitation and High temperature conditions for any of the molecules and the results were comparable to F0, in which all the values were in a range from 80 to 120% of binding.

### Example 10

### Evaluation of Stability of Pembrolizumab Formulations (DoE Approach)

Design of Experiments (DoE) uses a multifactorial approach employing statistical methodologies to both design and analyse an experimental process. In a ‘designed’ experiment a number of factors are simultaneously varied. The DoE approach studied herein consisted in three steps (DoE1, DoE2 and DoE3).

The Design of Experiments (DoE) was performed by using the Umetrics™ Suite of Data Analytics Solutions (MODDER DoE software, Sartorius). This software was able to analyze the statistical accuracy of the results.

Before starting with the DoE1 a previous study to identify the optimal pH was performed, formulating the antibody (pembrolizumab) in a histidine-acetate buffer in different pHs: 5.2, 5.4 and 5.8 and first evaluating the pH changes under different buffer strengths (10 mM, 20 mM, 30 mM) at 40° C. for 2 weeks. After that, one buffer strength/pH was selected and put under thermal stress at 40° C. for two weeks and analysed for its potential stability through different techniques (SEC, μDSC, DSF, CE-SDS NR, CE-SDS R, IEX or cIEF, data not shown). Following that, up to two buffers were selected for the DoE1 approach based on the pH determined before and the pKa of the buffer, also one or two excipients were chosen for each category.

A DoE1 (Design of Experiments) screening study was conducted to comparatively evaluate fifteen formulations and the impact of several factors to stabilize pembrolizumab formulations over time. The fifteen formulations included changes in buffer type, sugar/polyol type, amino acid type, antioxidant type and surfactant type. The formulations tested are shown in Table 3 below. Pembrolizumab antibody concentration was in all cases 25 mg/ml and pH between 5.4-5.5.

TABLE 3
Pembrolizumab formulations tested in the Optimal Design runs (DOE1).

| Exp | Buffer | Sugar/polyol | Surfactant | Amino | Antioxidant |
|---|---|---|---|---|---|
| Number | Type | Type | Type | Acid Type | Type |
| 1 | Acetate- | Dextran 40 | Poloxamer 188 | Arginine | Methionine |
| | Histidine | | | | |
| 2 | Acetate | Trehalose | Hydroxypropyl- | Arginine | Methionine |
| | | | B-cyclodextrin | | |
| 3 | Acetate | Mannitol | Poloxamer 188 | Proline | Methionine |
| 4 | Acetate | Dextran 40 | Poloxamer 188 | Proline | Methionine |
| 5 | Acetate- | Trehalose | Hydroxyproplyl- | Proline | Methionine |
| | Histidine | | B-cyclodextrin | | |
| 6 | Acetate | Trehalose | Poloxamer 188 | Non- | Methionine |
| | | | | aminoacid | |
| 7 | Acetate- | Mannitol | Hydroxyproplyl- | Non- | Methionine |
| | Histidine | | B-cyclodextrin | aminoacid | |
| 8 | Acetate- | Trehalose | Poloxamer 188 | Arginine | Non- |
| | Histidine | | | | antioxidant |
| 9 | Acetate | Mannitol | Hydroxyproplyl- | Arginine | Non- |
| | | | B-cyclodextrin | | antioxidant |
| 10 | Acetate- | Mannitol | Poloxamer 188 | Proline | Non- |
| | Histidine | | | | antioxidant |
| 11 | Acetate- | Trehalose | Hydroxyproplyl- | Proline | Non- |
| | Histidine | | B-cyclodextrin | | antioxidant |
| 12 | Acetate | Dextran 40 | Hydroxypropyl- | Proline | Non- |
| | | | B-cyclodextrin | | antioxidant |
| 13 | Acetate | Trehalose | Poloxamer 188 | Non- | Non- |
| | | | | aminoacid | antioxidant |
| 14 | Acetate- | Dextran 40 | Poloxamer 188 | Non- | Non- |
| | Histidine | | | aminoacid | antioxidant |
| 15 | Acetate | Dextran 40 | Hydroxyproplyl- | Non- | Non- |
| | | | B-cyclodextrin | aminoacid | antioxidant |

DoE1 allowed the selection of the optimal conditions buffer type, sugar/polyol type, surfactant type, amino acid type and antioxidant to be used for the subsequent DoE2 confirmation study described below.

### Preparation of the Pembrolizumab Samples

The pre-formulated drug substance (DS, pembrolizumab) was fully purified under Protein A chromatography, viral inactivation and several ion exchange chromatography steps. A buffer exchange by filtration with Amicon® Ultra Centrifugal Filters with a cut-off of 30 KDa (Merck) was performed. 10 diavolumes were passed through the protein sample to ensure full buffer exchange in the corresponding buffer. After that, a final reconcentration step was performed to achieve final concentration of 25 mg/ml. The required excipients were also added to the relevant buffer-exchanged DS material and pH adjusted to the target so as to obtain the compositions listed in Table 3.

DoE1 experimental results demonstrate that a positive impact on aggregation was observed with Mannitol, Trehalose, Methionine, and Histidine-acetate buffer and a negative impact on aggregation was observed for Dextran 40, Acetate buffer and non-antioxidant when maintained at 40° C. for up to 4 weeks (% AggT40C 4w), as shown in FIG. 10. The levels of aggregation and main peak were measured by SEC and represented in the graph of FIG. 10.

DoE1 experimental results on charge variants were measured and analyzed by IEX. The results demonstrate that a positive impact on charge variants (acidic species) was observed for Mannitol, Trehalose, Methionine, Histidine-acetate and Arginine, wherein a negative impact on charge variants (acidic species) was observed for Dextran 40 when maintained at 40° C. for up to 4 weeks (% Ac Sp T40C 4w), as shown in FIG. 11.

DoE1 outcome analysed by MODDER Software allowed to select eight formulations that were tested in a next DoE2 confirmation experimental run. The DoE2 study was conducted to comparatively evaluate the effect of Mannitol versus Trehalose and the impact of amino acid (Arginine versus Proline) in an Acetate-Histidine buffer with a Poloxamer 188 surfactant without an antioxidant (fixed excipients, the buffer, the surfactant and the antioxidant). The formulations tested in the DoE2 confirmation run are shown in Table 4 below. Pembrolizumab antibody concentration was in all cases 25 mg/ml.

TABLE 4
Pembrolizumab formulations tested in the confirmation Optimal Design runs

| Exp | Buffer | Sugar/polyol | Surfactant | Amino | Antioxidant |
|---|---|---|---|---|---|
| Number | Type | Type | Type | Acid Type | Type |
| 1 | Acetate- | Mannitol | Poloxamer 188 | Arginine | Non- |
| | Histidine | | | | antioxidant |
| 2 | Acetate- | Trehalose | Poloxamer 188 | Arginine | Non- |
| | Histidine | | | | antioxidant |
| 3 | Acetate- | Mannitol | Poloxamer 188 | Proline | Non- |
| | Histidine | | | | antioxidant |
| 4 | Acetate- | Trehalose | Poloxamer 188 | Proline | Non- |
| | Histidine | | | | antioxidant |
| 5 | Acetate- | Mannitol | Poloxamer 188 | Arginine | Non- |
| | Histidine | | | | antioxidant |
| 6 | Acetate- | Trehalose | Poloxamer 188 | Arginine | Non- |
| | Histidine | | | | antioxidant |
| 7 | Acetate- | Mannitol | Poloxamer 188 | Proline | Non- |
| | Histidine | | | | antioxidant |
| 8 | Acetate- | Trehalose | Poloxamer 188 | Proline | Non- |
| | Histidine | | | | antioxidant |

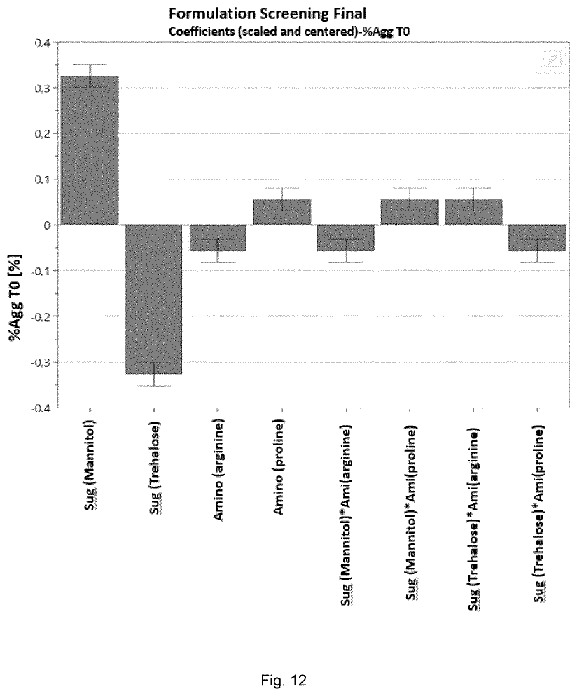

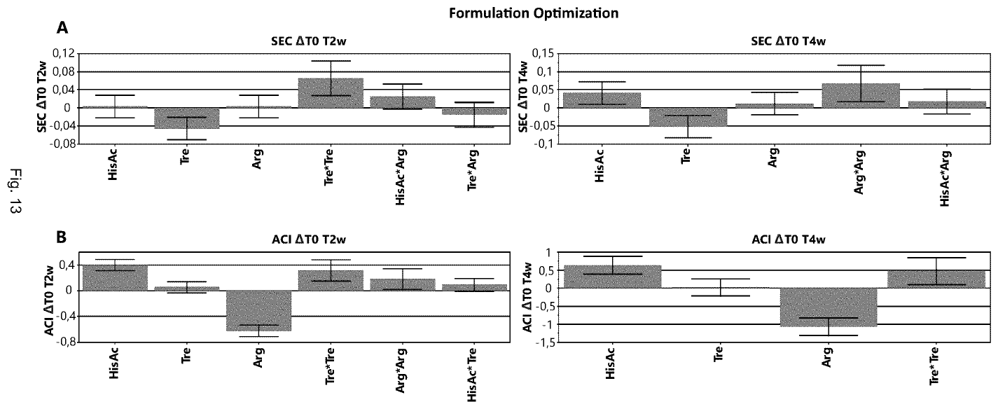

DoE2 outcome on aggregation were measured by SEC and demonstrate that slightly higher levels of aggregates were found in formulations containing Mannitol vs formulations containing Trehalose when maintained at 40° C. for up to 4 weeks (% Agg T40C 4w). Interestingly, at T0, after at least two of freeze/thaw cycles (frozen long-term effect) it was found that values of aggregates were higher (% Agg T0) for the formulations containing mannitol (negative impact) versus trehalose (positive impact), as shown in FIG. 12. No statistically significant variations in terms of aggregates upon thermal stress at 40° C. could be highlighted for the rest of factors evaluated, thus indicating that the different matrices tested led to invariant/negligible changes in the aggregation pattern.

The next step was a DoE3 optimization experiment to explore which ranges of concentration were behaving better in the final formulation. The excipients tested were, Histidine-acetate (5 mM, 27.5 mM and 50 mM), Trehalose (50 mM, 150 mM and 250 mM) and an amino acid (such as arginine) at 5 mM, 77.5 mM and 155 mM. Poloxamer 188 was used at a fixed concentration of 0.2%. The different formulations were studied from T0 up to 2 weeks and up to 4 weeks. FIGS. 13A and 13B show their increments in % for aggregates (SEC ΔT0 T2w n=17; DF (Degree of Freedom)=10; R=0.79 and ΔT0 T4w n=16; DF=10; R=0.7, confidence=0.95) and for charge variants (acidic species, ACI ΔT0 T2w n=16; DF=9; R2=0.98 and ACI ΔT0 T4w n=17; DF=12; R2=0.92 confidence=0.95). The results corroborate that trehalose has a positive impact in the level of aggregation and the presence of an amino acid (such as arginine) has a positive impact in the levels of acidic species. In particular, when Histidine-acetate is about 5 mM, trehalose is about 200 mM, and the amino acid (such as arginine) is about 110 mM. The levels of aggregation and main peak were measured by SEC, wherein the charge variants were measured and analyzed by IEX.

When combining all the responses obtained during the DoE3 approach, a formulation with optimal working concentrations (Sweet spot) for each of the excipients was suggested, considering the % levels of aggregation and the acid variants change. Good results were obtained when Histidine-acetate buffer was in the low ranges of the concentration used (˜5 mM), an amino acid (e.g., arginine or proline) in the high ranges of the concentration used (˜50 to 155 mM) and trehalose in the high ranges of the concentration used (˜85-250 mM), which allows to predict values of each excipient in the different candidate formulations. In particular, optimal working concentrations were found for formulations comprising histidine-acetate buffer at 5 mM, trehalose at 95 mM and arginine or proline at 155 mM, and furthermore for formulations comprising histidine acetate at 5 mM, trehalose at 200 mM and arginine or proline at 110 mM. As commented before, for all formulations tested, Poloxamer 188 was used at a fixed concentration of 0.2%.

### Example 11

### Comparative Data for F45′ and Reference Product (Keytruda)

TABLE 5
Selected formulation deriving from DoE3 compared with F0 and Keytruda.

| | | | | | Amino |
|---|---|---|---|---|---|
| | Antibody | Buffer | Surfactant | Sugar | acid |
| F45′ | Pembrolizumab | Histidine- | Poloxamer 188 | Trehalose | Arginine |
| | 25 mg/ml | acetate 5 mM | 0.2% | 95 mM | 155 mM |
| F0 | Pembrolizumab | Histidine | Polysorbate 80 | Sucrose | — |
| | 25 mg/ml | 10 mM | 0.02% | 70 mg/mL | |
| Keytruda | Pembrolizumab | Histidine | Polysorbate 80 | Sucrose | — |
| | 25 mg/ml | 10 mM | 0.02% | 70 mg/mL | |

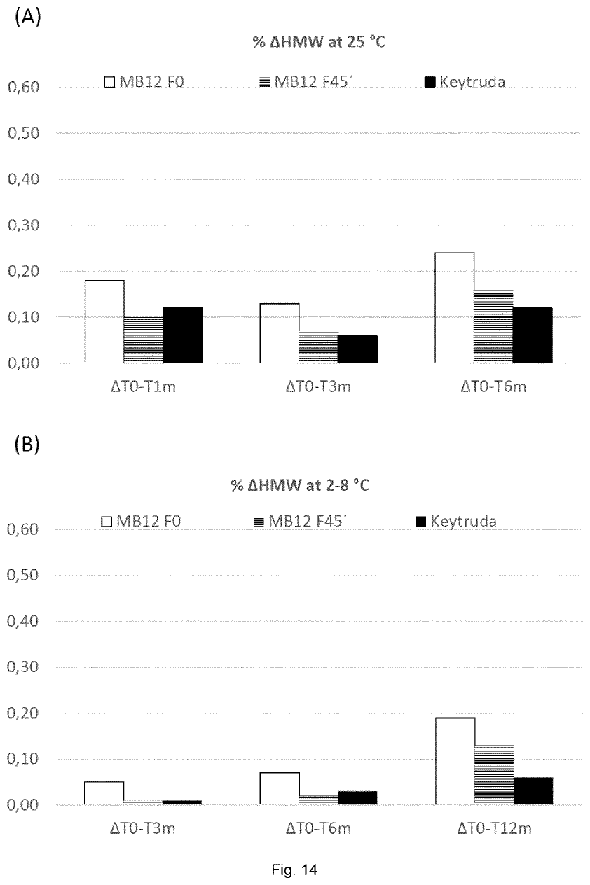

F45′ containing pembrolizumab at 25 mg/ml formulation deriving from DoE3, was studied together with pembrolizumab formulated in F0 (commercial formulation of the reference product) and Keytruda reference product. FIG. 14 (A) shows the % of high-molecular weight species or levels of aggregates rate (Δ% HMW) from time point 0 to 1 month, 3 months and 6 months time points (% ΔT0-T1m, T0-T3m, T0-T6m) at 25° C., measured by size exclusion chromatography (SEC, as described in example 4). At the three time points (1 month, 3 months and 6 months) pembrolizumab biosimilar, when formulated in F45′, is behaving in a similar way than the commercial product Keytruda and a less rate of aggregates is found when the pembrolizumab biosimilar is formulated in F0 (commercial formulation of Keytruda).

FIG. 14 (B) shows the % of high-molecular weight species or levels of aggregates measured by SEC rate (Δ% HMW) from time point 0 to time points 3 months, 6 months and 12 months (% ΔT0-T3m, T0-T6m, T0-T12m) at 2-8° C. Same performance can be seen when the rate of aggregation is carried out at 2-8° C. up to 12 months. The behavior of pembrolizumab when formulated in F45′ is similar to Keytruda commercial reference product and a less % of aggregation rate can be found when compared with pembrolizumab formulated in F0.

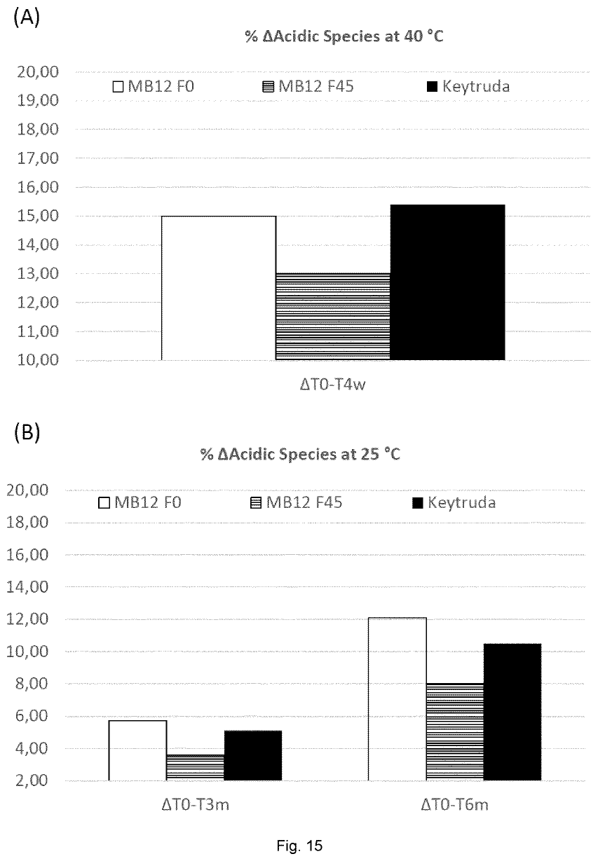

FIG. 15 (A) shows pembrolizumab biosimilar when formulated in F45′% of acidic species rate (% ΔAc), analyzed by IEX (as described in example 5) when maintaining the pembrolizumab formulations showed in Table 5 at a temperature of 40° C. for up to 4 weeks (ΔT0-T4w) and when maintaining at a temperature of 25° C. up to 3 months or up to 6 months. Pembrolizumab biosimilar formulated in F45′ composition is behaving better on regards the % of acidic species rate in all the settings when compared with F0 (pembrolizumab biosimilar in commercial formulation), and when compared with the reference product Keytruda. F45′ seems to keep better protected the antibody against the formation of aggregates and the increase of acid species.

Furthermore, in-use stability studies were designed to verify the product stability in in-use conditions. The aim is to provide stability assurance in real-life conditions for MB12, head-to-head with Keytruda.

Two types of exercises were carried out. First exercise, consisted in testing the stability of the molecule in the infusion bag during a certain period of time, to check the maximum time that the molecule could be kept it in the bag before the infusion. Second exercise consisted in testing the stability of the molecule during the infusion bag, replicating real life conditions for the administration of the drugs. In both cases, the samples were prepared at 1 mg/ml in 100 ml infusion bag 0.9% sodium chloride, as it is recommended in the EU, US and Japan leaflets. The infusion set exercise was carried out in 30 min keeping a flow of 3.33 ml/min, that corresponds to 66 drops per minute under the flow hood. Placebos were prepared using the corresponding formulation buffer mainly for particles counting techniques.

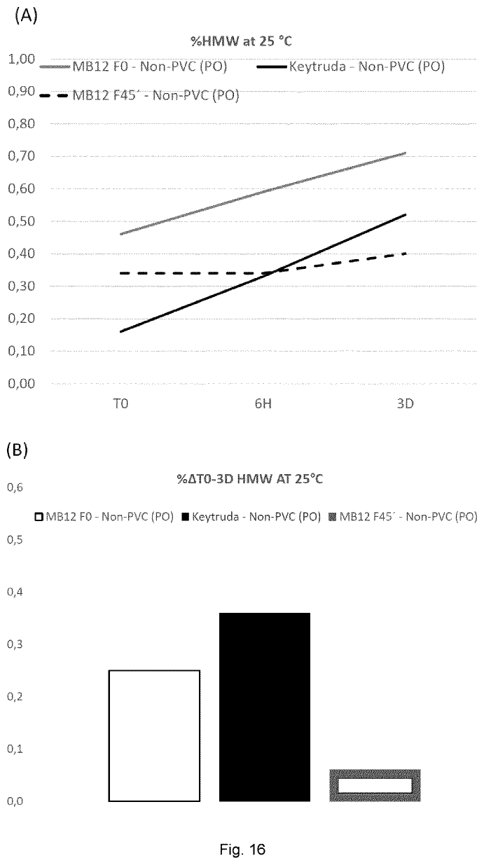

Stability in infusion bags filled with NaCl were tested for both materials (MB12 and Keytruda) at 25° C. and 2-8° C. by SEC and by Micro Fluid Imaging (MFI). Three different bags were tested ((PVC-DEHP, EVA and Non-PVC (PO)). For PVC-DEHP and EVA bags data confirmed that both materials were having similar behavior (data not shown) so confirmation of compatibility of the product with these material of bags and infusion sets in in-use conditions of MB12 product can be made. FIG. 16 (A) and (B) shows the % of high-molecular weight species or levels of aggregates (% HMW) measured by SEC when incubating the pembrolizumab formulations showed in Table 5 at a temperature of 25° C. for up to 3 days (3D) in Non-PVC (PO) bags. An increase in aggregates was observed for both molecules MB12 biosimilar in F0 and Keytruda in the Non-PVC (PO) bags after the 6 hours up to 3 days at 25° C., data that was expected considering the instructions found in the label of Keytruda, where it is demonstrated in-use stability of 6 h at 25° C., reaching 0.35% and 0.25% of aggregates, respectively, wherein the MB12 in F45′ formulation is not reaching 0.1% suggesting a better stability when the product is formulated in F45′ than in F0, but also a much higher stability and longer (up to 3 days compared with 6 hours) in aggregates when compared with the reference product (Keytruda).

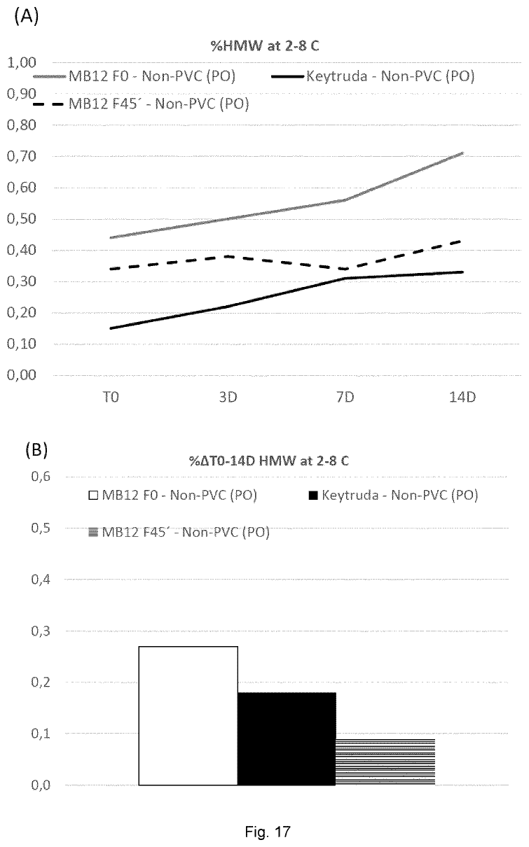

Increase in the level of aggregation was also observed at 2-8° C. at 14 days for MB12 and Keytruda, data that was expected considering the instructions found in the label of Keytruda, where it is mentioned that Keytruda shows an in-use stability up to 3 days at 2-8° C. The levels reached 0.27% when MB12 is formulated in F0, 0.39% in Keytruda and 0.09% when MB12 is formulated in F45′ formulation (FIG. 17). Thus, F45′ is showing a longer (up to 14 days) and lower level of aggregates at 2-8° C. This confirms the increased stability and protection of the molecule in Non-PVC bags at 25° C. up to 3 days and in 2-8° C. up to 14 days when formulated in F45′ compared with MB12 formulated in F0 and compared with the reference product Keytruda.

### Subvisible Particles Number Analyzed by MFI (Micro-Flow Imaging)

Subvisible particles number is a quality attribute in pharmaceuticals. The biopharmaceutical development of products based on proteins or monoclonal antibodies can lead to the formation of aggregates and/or particles, in addition to the presence of contaminants (such as silicone microdroplets, cellulose fibers), due to the potential problem that these particles or contaminants regulatory agencies require their optimal characterization. Harmonized protocols with European and Japanese standards set limits on the number of acceptable particles for parenteral products such as monoclonal antibody injectables. Injectables must be essentially free of visible particles and must contain small amounts of subvisible particles (SVPs) within the ranges: ≤6000 for particles ≥10 μm and ≤600 for particles ≥25 μm. MFI technology is based on the use of dynamic images that allow direct detection of particles. Allowing rapid quantification of the size and shape of thousands of particles per sample. This technique will allow us to evaluate during our formulation development different issues related to the stability and quality of our product. As well as the stability between different molecules, the impact on the change of formulation or, for example, on storage conditions. The samples were analyzed using an MFI 5100/5200 Flow Microscope, 2011 and a micro-flow imaging MFI view software, protein simple, 2014.

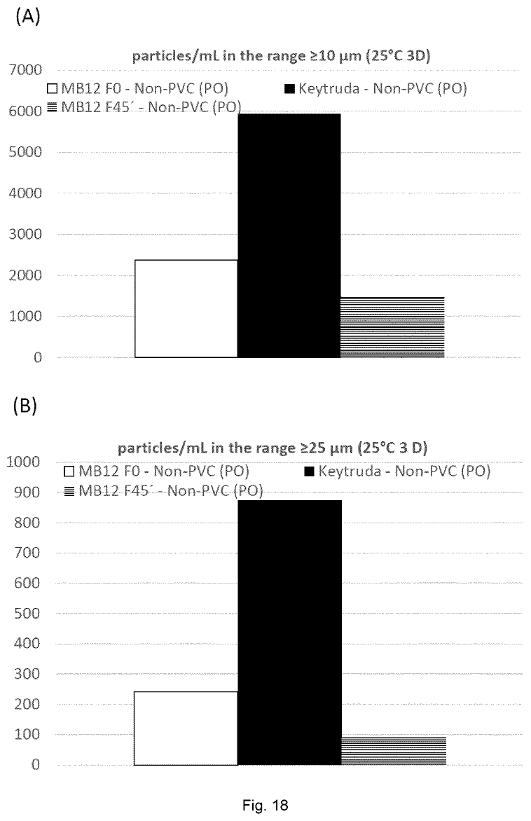

Number of subvisible particles in the solution was analyzed by MFI (FIG. 18 (A) and (B)). Higher number of subvisible particles was observed in the bags with the Keytruda material compared with the MB12 product (regardless the formulation used, F0 or F45). The infusion bags non-PVC (PO) is producing a greater number of subvisible particles (≥10 μm and ≥25 μm) when the reference product (Keytruda) is use compared with pembrolizumab biosimilar regardless the formulation used F0 or F45′ for particles/mL in the range ≥10 μm and in the range ≥25 μm.

### Example 12

### Stability of High Concentrated MB12 Antibody in Selected Formulation

TABLE 6
Formulations used to study stability of high concentrated MB12 antibody.

| | | | | | Amino |
|---|---|---|---|---|---|
| | Antibody | Buffer | Surfactant | Sugar | acid |
| F45′ | Pembrolizumab | Histidine- | Poloxamer 188 | Trehalose | Arginine |
| | 25 mg/ml | acetate | 0.2% | 95 mM | 155 mM |
| | | 5 mM | | | |
| F0 | Pembrolizumab | Histidine | Polysorbate 80 | Sucrose | — |
| | 25 mg/ml | 10 mM | 0.02% | 204 mM | |
| Keytruda | Pembrolizumab | Histidine | Polysorbate 80 | Sucrose | — |
| | 25 mg/ml | 10 mM | 0.02% | 204 mM | |
| F48′ | Pembrolizumab | Histidine- | Poloxamer 188 | Trehalose | Arginine |
| | 165 mg/ml | acetate | 0.2% | 95 mM | 155 mM |
| | | 5 mM | | | |
| F49′ | Pembrolizumab | Histidine | — | Trehalose | Glycine |
| | 165 mg/ml | 25 mM | | 100 mM | 1.6 mM |

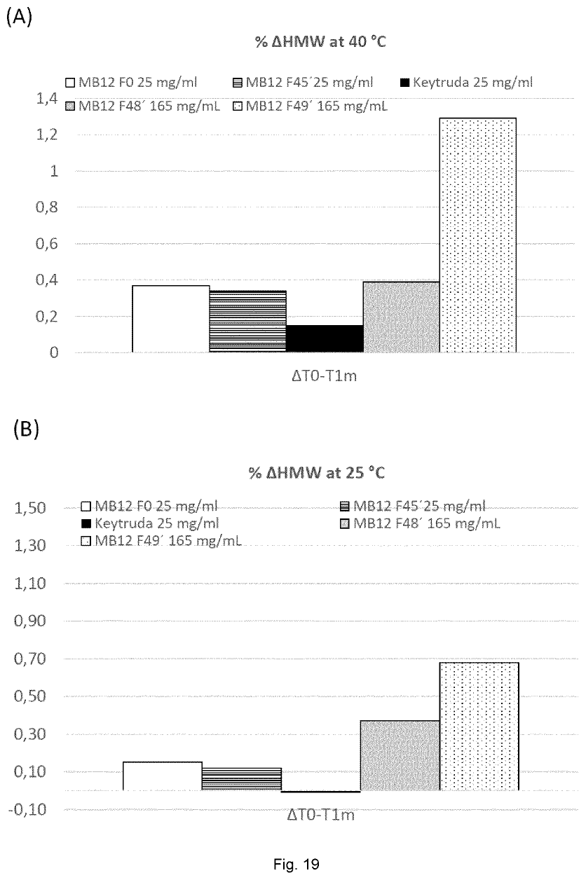

Highly concentrated antibody formulations were studied, to evaluate the possibility of using them for subcutaneous administration. However, the higher concentrated antibody formulations may have an impact in other properties of the composition which would be undesirable, e.g., low injectability due to increased viscosity and increased aggregation. The formulations described in table 6 were assessed by SEC, in order to evaluate the % of high-molecular weight species or levels of aggregates rate (Δ% HMW) when incubating from time point 0 to time point 1 month (% ΔT0-T1m) at 40° C. (FIG. 19A), at 25° C. (FIG. 19 B) and at 2-8° C. (data not shown).

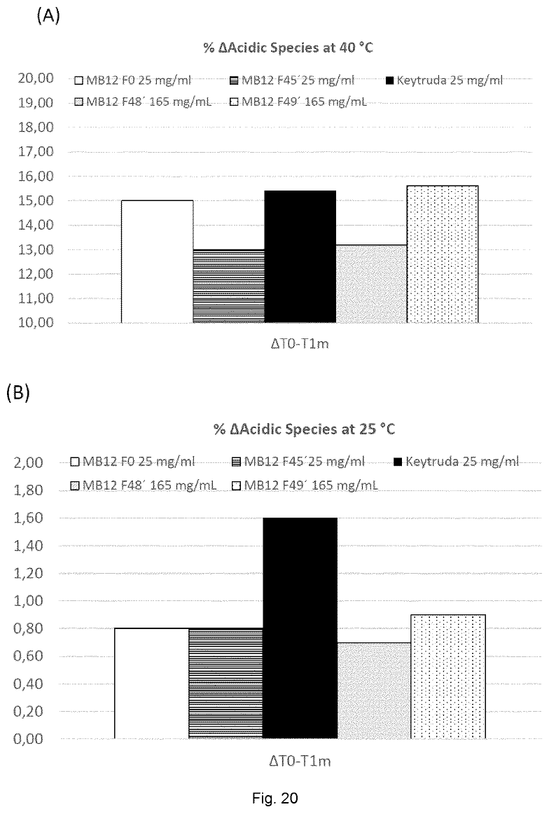

Data shown in FIG. 19A demonstrate that F48′ (pembrolizumab biosimilar at 165 mg/mL) formulation after 1 month at 40° C. (stress temperature) maintains the level of aggregation (% of high molecular weight species) at the same rate than F45′ (pembrolizumab biosimilar at 25 mg/mL) and F0 (pembrolizumab biosimilar at 25 mg/mL), and that the rate of aggregates is three time less than when the product at 165 mg/ml is formulated in F49′. Same results trend is observed at 25° C. (FIG. 19 B). No changes between all formulations tested, according to table 6, were found when incubated at 2-8° C. (data not shown). However, there is a much clear effect in protection when these formulations, described in table 6, are incubated at 40° C. (FIG. 20A), at 25° C. (FIG. 20 B) and at 2-8° C. (data not shown) and the % acidic species rate is measured by IEX. Data shown in FIG. 20A demonstrate that F48′ (pembrolizumab biosimilar at 165 mg/mL) formulation after 1 month at 40° (stress temperature) maintains the % acidic species rate at the same rate than F45′ (pembrolizumab biosimilar at 25 mg/mL) and being in both cases less than F0 (pembrolizumab biosimilar at 25 mg/mL), Keytruda and F49′ (pembrolizumab biosimilar at 165 mg/mL). When the % acidic species rate is measured at 25° C. at 1 month (FIG. 20 B), the effect on protecting the molecule against the formation of acid species is maintained for F48′ (pembrolizumab biosimilar at 165 mg/mL) particularly when compared with F49′ (pembrolizumab biosimilar at 165 mg/mL) and the reference product (Keytruda at 25 mg/mL).

### Viscosity Data by Uncle Platform

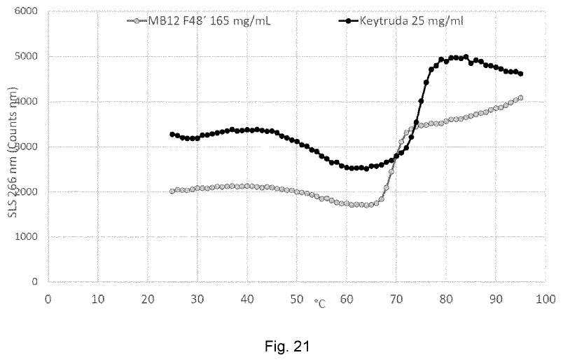

It is important to evaluate the increment in viscosity when the antibody is highly concentrated. The uncle equipment is a platform that allows determining several critical parameters in the stability of a biomolecule using only one instrument. To do this, this equipment combines the capacity of temperature control in a wide range (15°-95° C.) with static light scattering (SLS). Thanks to this combination, the uncle platform equipment makes possible to determine critical parameters in the stability of biomolecules such as the viscosity. For the analysis of the data generated by the uncle platform, the Uncle Analysis Software 5.03 and Uncle Instrument Client Software 5.03 were used. SLS measurements allow differentiation of the size of the particles and how those populations progress when a temperature ramp is applied. It allows us to determine the temperature of appearance of aggregates precisely. FIG. 21 shows the similar profile of increasing viscosity of the compositions plotted when the temperature rises to 95° C. F48 composition (pembrolizumab biosimilar at 165 mg/mL) is comparable with the behavior shown by the reference product Keytruda (25 mg/mL).

### Example 13

Freeze-Thaw Stability Pembroluzimab at 165 mg/ml

Results: F48 pembrolizumab at 165 mg/ml formulation is stable after a freeze/thaw process. The product at 165 mg/ml can be frozen without a significant increase of aggregation level which is a prerequisite for a long-term storage of e.g. drug substance.

TABLE 7
Freeze-thaw stability pembrolizumab 165 mg/ml
F&T DATA (Levels of aggregates)

| | % HMW | % HMW |
|---|---|---|
| | T0 | After F&T |
| F48 | 0.36 | 0.42 |
| (165 mg/mL) | | |

Methods: F48 pembrolizumab at 165 mg/mL was tested for stability against freeze/thaw stress and analyzed with regard to the presence of HMWS by SEC-HPLC (method described in example 4). F48 pembrolizumab at 165 mg/ml was analyzed before and after a freezing process to <−70° C. by SEC-HPLC.

### Example 14

Functionality Characteristics of Pembrolizumab at 165 mg/mL Injection

TABLE 8
Functionality characteristics of pembrolizumab
at 165 mg/mL injection

| | Pembrolizumab Injection at 165 mg/ml | | | |
|---|---|---|---|---|
| | NEOPAK (N) | | HYPAK (N) | |
| | | Std. | | Std. |
| | Nominal | Deviation | Nominal | Deviation |
| Parameter | Value | (σ) | Value | (σ) |
| Gliding force | 9.1 | 0.2 | 9.1 | 0.6 |
| Break Loose force | 5.8 | 0.7 | 4.8 | 1.4 |

Results: Quantification of break loose force and gliding force when using a prefilled syringe suitable for subcutaneous injection. Break loose force and gliding force show no significant differences for NEOPAK and HYPAK syringes. The results (see Table 8) show a break-loose and gliding force (average) below 15 N for F48 pembrolizumab at 165 mg/ml when expelling the content. These values allow an easy and safe subcutaneous injection of the highly concentrated solutions into the patient.

Methods: The study was performed in 6 samples of Pembrolizumab Injection F48 high concentration:

| | Units to | | |
|---|---|---|---|
| | be tested | Syringe | |
| Device | (ud) | Model | ID |
| Prefilled | 3 | NEOPAK | 122003.580 |
| Syringe | | | 122003.581 |
| | | | 122003.582 |
| Prefilled | 3 | HYPAK | 122003.583 |
| Syringe | | | 122003.584 |
| | | | 122003.585 |

Samples are received in bulk, the plunger not assembled to the stopper. The Zwick Tensile is employed for forces measurement. The prefilled syringes are received in bulk. The normal procedure when the samples are received with the plunger dis-assembled is to test needle shield removal force (data not shown), storing for one week for the break loose force to be recovered after sample manipulation and afterwards testing break loose and gliding force test.

What is claimed is:

1. A formulation comprising an antibody, a buffer, an amino acid and trehalose, having a pH between 5.0 to 7.0.

2-99. (canceled)

100. The formulation according to claim 1, wherein the buffer is an 5 mM to 50 mM histidine buffer.

101. The formulation according to claim 100, wherein the histidine buffer is a histidine-acetate buffer.

102. The formulation according to claim 1, wherein the amino acid is selected from the group consisting of glycine, histidine, lysine, arginine, proline, serine, methionine and alanine.

103. The formulation according to claim 1, wherein the amino acid is at a concentration of about 1-155 mM.

104. The formulation according to claim 102, wherein the amino acid is arginine or proline at a concentration of about 5-155 mM.

105. The formulation according to claim 104, wherein the arginine or proline is at a concentration of about 110 mM.

106. The formulation according to claim 102, wherein the amino acid is glycine at a concentration of 1 to 3 mM.

107. The formulation according to claim 106, wherein the glycine is at a concentration of 1.6 mM.

108. The formulation according to claim 1, wherein the trehalose is at a concentration of 50 to 250 mM.

109. The formulation according to claim 108, wherein the trehalose is at a concentration of 95 mM.

110. The formulation according to claim 1, wherein the pH of the formulation is pH 5 to 6.

111. The formulation according to claim 1, further comprising a surfactant.

112. The formulation according to claim 111, wherein the surfactant is poloxamer 188 and it is found at a concentration of between 0.01 and 0.2% (w/v).

113. The formulation according to claim 1, wherein the antibody is at a concentration of 10-100 mg/ml.

114. The formulation according to claim 1, wherein the antibody is Palivizumab.

115. The formulation according to claim 114, wherein Palivizumab is at a concentration of 100 mg/ml and the amino acid is glycine at a concentration of 1.6 mM, the buffer is histidine at a concentration of 25 mM and trehalose at a concentration of 100 mM.

116. The formulation according to claim 1, wherein the antibody is Pembrolizumab or Nivolumab.

117. The formulation according to claim 116, wherein the antibody is Pembrolizumab at concentration of 25 mg/ml and the amino acid is arginine or proline at a concentration of 5-155 mM, the buffer is histidine-acetate at a concentration of 5-50 mM and trehalose is at a concentration of 50-250 mM.

118. The formulation according to claim 116, wherein the antibody is Nivolumab at a concentration of 10 mg/ml and the amino acid is arginine or proline at a concentration of 5-155 mM, the buffer is histidine-acetate at a concentration of 5-50 mM and trehalose is at a concentration of 50-250 mM.

119. A method for the prevention and/or treatment of a subject infected by Respiratory Syncytial Virus (RSV) comprising administering to the subject the formulation according to claim 1.

120. A method for preparing a stabilized antibody-containing composition comprising formulating the antibody in a composition comprising a buffer, an amino acid and trehalose, and wherein the formulation has a pH of 5.0 to 7.0.
